(19) United States
(12) Patent Application Publication
(10) Pub. No.: US 2008/0221210 A1
(43) Pub. Date: Sep. 11, 2008

(54) Thyroid Receptor Agonists

(75) Inventors: Ana Maria Garcia Collazo, Stockholm (SE); Thomas Anders Wilson Norin, Sodertalje (SE); Neerai Garg, Tumba (SE); Anton Joakim Lofstedt, Uppsala (SE); Tomas Fredrik Hansson, Tumba (SE); Lars Jesper Hallberg, Stockholm (SE); Peter Brandt, Uppsala (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(73) Assignee: Karo Bio AB, Huddinge (SE)

(21) Appl. No.: 10/593,928

(22) PCT Filed: Mar. 22, 2005
(86) PCT No.: PCT/EP05/03033

§ 371 Date: Apr. 28, 2008

(30) Foreign Application Priority Data
Mar. 22, 2004 (GB) 0406380.6

(51) Int. Cl. A61K 31/216; C07C 229/36; A61P 9/00; A61K 31/197

(52) U.S. Cl. 514/539; 562/449; 514/563; 560/41; 560/43

(57) ABSTRACT

The invention provides compounds of formula (I) or a pharmaceutically acceptable ester, amide, solvate or salt thereof, including a salt of such an ester or amide, and a solvate of such an ester, amide or salt. The invention also provides the use of such compounds in the treatment or prophylaxis of a condition mediated by a thyroid receptor. Formula (I), wherein R¹, R², n, Y, Y′, R³, R⁴, W and R⁵ are as defined in the specification.

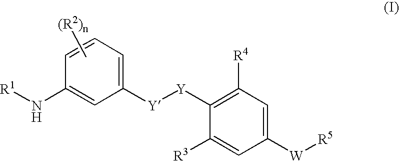

### FIELD OF THE INVENTION

The present invention relates to compounds which are agonists or partial agonists of the thyroid receptor and the use of such compounds for therapeutic purposes

### BACKGROUND OF THE INVENTION

While the extensive role of thyroid hormones in regulating metabolism in humans is well recognized, the discovery and development of new specific drugs for improving the treatment of hyperthyroidism and hypothyroidism has been slow. This has also limited the development of thyroid agonists and antagonists for treatment of other important clinical indications, such as hypercholesterolemia, dyslipidemia, obesity, diabetes, atherosclerosis and cardiac diseases.

Thyroid hormones affect the metabolism of virtually every cell of the body. At normal levels, these hormones maintain body weight, metabolic rate, body temperature and mood, and influence blood levels of serum lipoproteins. Thus, in hypothyroidism there is weight gain, high levels of LDL cholesterol, and depression. In hyperthyroidism, these hormones lead to weight loss, hypermetabolism, lowering of serum LDL cholesterol levels, cardiac arrhythmias, heart failure, muscle weakness, bone loss in postmenopausal women, and anxiety.

Thyroid hormones are currently used primarily as replacement therapy for patients with hypothyroidism. Therapy with L-thyroxine returns metabolic functions to normal and can easily be monitored with routine serum measurements of levels of thyroid-stimulating hormone (TSH), thyroxine (3,5,3′,5′-tetraiodo-L-thyronine, or T₄) and triiodothyronine (3,5,3′-triiodo-L-thyronine, or T₃). However, replacement therapy, particularly in older individuals, may be restricted by certain detrimental effects from thyroid hormones.

In addition, some effects of thyroid hormones may be therapeutically useful in non-thyroid disorders if adverse effects can be minimized or eliminated. These potentially useful influences include for example, lowering of serum LDL levels, weight reduction, amelioration of depression and stimulation of bone formation. Prior attempts to utilize thyroid hormones pharmacologically to treat these disorders have been limited by manifestations of hyperthyroidism, and in particular by cardiovascular toxicity.

Furthermore, useful thyroid agonist drugs should minimize the potential for undesired consequences due to locally induced hypothyroidism, i.e. sub-normal levels of thyroid hormone activity in certain tissues or organs. This can arise because increased circulating thyroid hormone agonist concentrations may cause the pituitary to suppress the secretion of thyroid stimulating hormone (TSH), thereby reducing thyroid hormone synthesis by the thyroid gland (negative feedback control). Since endogenous thyroid hormone levels are reduced, localized hypothyroidism can result wherever the administered thyroid agonist drug fails to compensate for the reduction in endogenous hormone levels in specific tissues.

Development of specific and selective thyroid hormone receptor ligands, particularly agonists of the thyroid hormone receptor, is expected to lead to specific therapies for these common disorders, while avoiding the cardiovascular and other toxicity of native thyroid hormones. Tissue-selective thyroid hormone agonists may be obtained by selective tissue uptake or extrusion, topical or local delivery, targeting to cells through other ligands attached to the agonist and targeting receptor subtypes. Tissue selectivity can also be achieved by selective regulation of thyroid hormone responsive genes in a tissue specific manner.

Accordingly, the compounds that are thyroid hormone receptor ligands, particularly selective agonists of the thyroid hormone receptor, are expected to demonstrate a utility for the treatment or prevention of diseases or disorders associated with thyroid hormone activity, for example: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

### SUMMARY OF THE INVENTION

The present invention provides a compound of formula (I) or a pharmaceutically acceptable ester, amide, solvate or salt thereof, including a salt of such an ester or amide, and a solvate of such an ester, amide or salt,

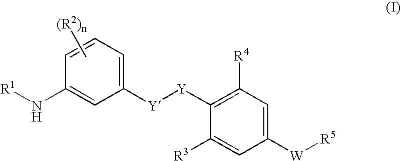

wherein:
R¹ is selected from hydrogen, C₁₋₈ alkyl, C₂₋₈ alkenyl, C₂₋₈ alkynyl, C₃₋₈ cycloalkyl and C₃₋₈ cycloalkyl-C₁₋₃ alkyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy; said cycloalkyl groups or portions of groups optionally being substituted with 1, 2 or 3 groups independently selected from halogen, hydroxy, C₁₋₄ alkyl, C₂₋₄ alkenyl, C₂₋₄ alkynyl, methoxy, halomethoxy, dihalomethoxy, trihalomethoxy, haloC₁₋₄ alkyl, dihaloC₁₋₄ alkyl, and trihaloC₁₋₄ alkyl;
Each R² is independently selected from halogen, mercapto, nitro, cyano, C₁₋₄ alkoxy, —CO₂R^{c}, —CONHR^{c}, —CHO, —SO₂R⁶, —SO₂NHR⁶, C₁₋₄ alkyl, C₂₋₄ alkenyl, C₂₋₄ alkynyl, NHR¹ and N(R¹)₂, said alkyl, alkenyl, alkynyl or alkoxy groups or portions of groups optionally being substituted with 1, 2 or 3 groups selected from halogen, hydroxy, C₁₋₄ alkoxy, C₁₋₄ alkylthio, mercapto, nitro, cyano, halomethoxy, dihalomethoxy, and trihalomethoxy;
n is 0, 1, 2 or 3;
Y and Y′ together are —C(R^{a′})═C(R^{a′})—,
or alternatively Y and Y′ are independently selected from oxygen, sulphur and —CH(R^{a})—, with the proviso that at least one of Y and Y′ is —CH(R^{a})— and the further proviso that when one of Y and Y′ is oxygen or sulphur, then R^{a} is hydrogen, halogen, C₁₋₄ alkyl, C₂₋₄ alkenyl, C₂₋₄ alkynyl, fluoromethyl, difluoromethyl, or trifluoromethyl;
R^{a} is selected from hydrogen, halogen, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio and thiotrifluoromethyl;
R^{a′} is selected from hydrogen, halogen, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio and thiotrifluoromethyl;
R³ and R⁴ are independently selected from halogen, C₁₋₄ alkyl, C₂₋₄ alkenyl, C₂₋₄ alkynyl, fluoromethyl, difluoromethyl, trifluoromethyl, C₁₋₄ alkoxy, fluoromethoxy, difluoromethoxy, trifluoromethoxy, methylthio, fluoromethylthio, difluoromethylthio and trifluoromethylthio;
W is selected from C₁₋₃ alkylene, C₂₋₃ alkenylene, C₂₋₃ alkynylene, N(R^{b})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NH(CO)—C₀₋₃ alkylene and C₁₋₃ alkyleneC(O)NH—C₁₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;

R^{b} is selected from hydrogen, hydroxy, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, and trifluoromethoxy;

R⁵ is selected from —CO₂R^{c}, —PO(OR^{c})₂, —PO(OR^{c})NH₂, —SO₂OR^{c}, —COCO₂R^{c}, CONR^{c}OR^{c}, —SO₂NHR^{c}, —NHSO₂R^{c′}, —CONHSO₂R^{c}, and —SO₂NHCOR^{c};

Each R^{c} is independently selected from hydrogen, C₁₋₄ alkyl, C₂₋₄ alkenyl and C₂₋₄ alkynyl;
R^{c′} is selected from R^{c}, C₅₋₁₀ aryl and C₅₋₁₀ aryl substituted with 1, 2 or 3 groups independently selected from amino, hydroxy, halogen or C₁₋₄ alkyl;
with the proviso that when simultaneously n=0, R³═R⁴═Br, Y═O, Y′═CH₂, W═CH₂—CH₂ and R⁵═CO₂H, then R₁ is not ethyl or hydrogen.

Compounds of the invention have surprisingly been found to be ligands of the thyroid receptor, in particular agonists or partial agonists of the thyroid receptor. The compounds accordingly have use in the treatment or prophylaxis of conditions associated with thyroid receptor activity.

### DETAILED DESCRIPTION OF THE INVENTION

The compounds of formula (I) may contain chiral (asymmetric) centres or the molecule as a whole may be chiral. The individual stereoisomers (enantiomers and diastereoisomers) and mixtures of these are within the scope of the present invention.

Preferably, R¹ is selected from methyl, i-propyl, n-propyl, i-butyl, n-butyl, sec-butyl, t-butyl, C₂₋₄ alkenyl, C₃₋₆ cycloalkyl-C₁₋₃ alkyl and substituted C₁₋₄ alkyl. More preferably, R¹ is selected from C₂, alkenyl, and C₃₋₆ cycloalkyl-C₁₋₃ alkyl and substituted C₁₋₄ alkyl. Preferred substituents for said alkyl or alkenyl include groups independently selected from halogen, methoxy, halomethoxy, dihalomethoxy, and trihalomethoxy. Preferred substituents for said cycloalkyl include halogen, methyl, ethyl, methoxy, halomethoxy, dihalomethoxy, and trihalomethoxy. More preferred substituents are halogens.

When n=0, R¹ is more preferably selected from cyclopropyl-methyl, methyl, i-propyl, i-butyl, sec-butyl, cyclobutyl and cyclobutyl-methyl.

When n=1, 2 or 3, R¹ is preferably selected from methyl, ethyl, i-propyl, n-propyl, i-butyl, sec-butyl, n-butyl, t-butyl, C₂₋₄ alkenyl, C₃₋₆ cycloalkyl, C₃₋₆ cycloalkyl-C₁₋₃ alkyl and substituted C₁₋₄ alkyl. More preferably, R¹ is selected from C₂₋₄ alkenyl, and C₃₋₆ cycloalkyl-C₁₋₃ alkyl and substituted C₁₋₄ alkyl. Preferred substituents for said alkyl or alkenyl include groups independently selected from halogen, methoxy, halomethoxy, dihalomethoxy, and trihalomethoxy. Preferred substituents for said cycloalkyl include halogen, methyl, ethyl, methoxy, halomethoxy, dihalomethoxy, and trihalomethoxy. More preferred substituents are halogens. More preferably, R¹ is selected from cyclopropyl-methyl, methyl, ethyl, i-propyl, sec-butyl, i-butyl, cyclobutyl and cyclobutyl-methyl; particularly ethyl.

When R³ and R⁴ are not both simultaneously Br, R¹ is preferably selected from methyl, ethyl, i-propyl, n-propyl, i-butyl, sec-butyl, n-butyl, t-butyl, C₂₋₄ alkenyl, C₃₋₆ cycloalkyl, C₃₋₆ cycloalkyl-C₁₋₃ alkyl and substituted C₁₋₄ alkyl. More preferably, R¹ is selected from C₂₋₄ alkenyl, and C₃₋₆ cycloalkyl-C₁₋₃ alkyl and substituted C₁₋₄ alkyl. Preferred substituents for said alkyl or alkenyl include groups independently selected from halogen, methoxy, halomethoxy, dihalomethoxy, and trihalomethoxy. Preferred substituents for said cycloalkyl include halogen, methyl, ethyl, methoxy, halomethoxy, dihalomethoxy, and trihalomethoxy. More preferred substituents are halogens. More preferably, R¹ is selected from cyclopropyl-methyl, methyl, ethyl, i-propyl, sec-butyl, i-butyl, cyclobutyl and cyclobutyl-methyl; particularly ethyl.

When simultaneously n=1, 2 or 3 and R³ and R⁴ are both not Br, R¹ is preferably selected from methyl, ethyl, i-propyl, n-propyl, i-butyl, sec-butyl, n-butyl, t-butyl, C₂₋₄ alkenyl, C₃₋₆ cycloalkyl, C₃₋₆ cycloalkyl-C₁₋₃ alkyl and substituted C₁₋₄ alkyl. More preferably, R¹ is selected from C₂₋₄ alkenyl, C₃₋₆ cycloalkyl-C₁₋₃ alkyl, and substituted C₁₋₄ alkyl. Preferred substituents for said alkyl or alkenyl include groups independently selected from halogen, methoxy, halomethoxy, dihalomethoxy, and trihalomethoxy. Preferred substituents for said cycloalkyl include halogen, methyl, ethyl, methoxy, halomethoxy, dihalomethoxy, and trihalomethoxy. More preferred substituents are halogens. More preferably, R¹ is selected from cyclopropyl-methyl, methyl, ethyl, i-propyl, sec-butyl, i-butyl, cyclobutyl and cyclobutyl-methyl; particularly ethyl.

R² is preferably selected from halogen, C₁₋₂ alkyl, C₂₋₃ alkenyl, C₂₋₃ alkynyl, C₁₋₂ alkoxy, haloC₁₋₂ alkyl, dihaloC₁₋₂ alkyl, and trihaloC₁₋₂ alkyl. More preferably, R² is selected from halogen, methyl, trifluormethyl, difluoromethyl and fluoromethyl. When R² is a halogen, it is preferably selected from bromine, chlorine and fluorine, especially chlorine.

Preferably n is 0, 1 or 2. More preferably n is 1 or 2.

When R¹ is ethyl, n is preferably 1, 2 or 3. When R³ and R⁴ are both simultaneously bromine, n is preferably 1, 2 or 3. When simultaneously R¹ is ethyl, and R³ and R⁴ are both bromine, n is preferably 1, 2 or 3.

When R¹ is not ethyl, n is preferably 0. When R³ and R⁴ are not both simultaneously bromine, n is preferably 0. When simultaneously R¹ is not ethyl, R³ is not bromine and R⁴ is not bromine, n is preferably 0.

Preferably, Y and Y′ are independently selected from oxygen, sulphur or —CH(R^{a})—, with the proviso that at least one of Y and Y′ is —CH(R^{a})— and the further proviso that when one of Y and Y′ is oxygen or sulphur, then R^{a} is hydrogen, halogen, C₁₋₄ alkyl, C₂₋₄ alkenyl, C₂₋₄ alkynyl, fluoromethyl, difluoromethyl, trifluoromethyl. More preferably, Y is O or S, and Y′ is CH(R^{a}). Most preferably, Y is O and Y′ is CH(R^{a}).

In a second preferred embodiment, Y and Y′ together are —C(R^{a′})═C(R^{a′})—.

In another preferred embodiment, Y and Y′ together are —C(R^{a′})═C(R^{a′})— or CH(R^{a′})—CH(R^{a′})—, or alternatively Y is O or S, and Y′ is —CH(R^{a})—. In a further preferred embodiment, Y and Y′ together are —C(R^{a′})═C(R^{a′})—, or alternatively Y is O and Y′ is —CH(R^{a})—. Preferably, Y and Y′ together are —C(R^{a′})═C(R^{a′})—, CH(R^{a′})—CH(R^{a′})—, or —O—CH(R^{a})—.

R^{a} is preferably selected from hydrogen, halogen, C₁₋₂ alkyl, fluoromethyl, difluoromethyl and trifluoromethyl. More preferably, R^{a} is selected from hydrogen, halogen and C₁₋₂ alkyl. Most preferably, R^{a} is hydrogen.

R^{a′} is preferably selected from hydrogen, halogen, C₁₋₂ alkyl, fluoromethyl, difluoromethyl and trifluoromethyl. More preferably, R^{a′} is selected from hydrogen, halogen and C₁₋₂ alkyl. Most preferably, R^{a′} is hydrogen.

R³ and R⁴ are preferably independently selected from halogen, C₁₋₄ alkyl, fluoromethyl, difluoromethyl and trifluoromethyl. More preferably, R³ and R⁴ are independently selected from halogen, methyl, fluoromethyl, difluoromethyl and trifluoromethyl. Amongst the halogens, there are preferred bromine, chlorine and fluorine, especially bromine and chlorine, in particular bromine. When R¹ is ethyl, one of R³ or R⁴ is preferably chlorine; when R¹ is ethyl, R³ and R⁴ are preferably both chlorine. When R¹ is ethyl and n=0, one of R³ or R⁴ is preferably chlorine; when R¹ is ethyl and n=0, R³ and R⁴ are preferably both chlorine.

R³ and R⁴ may simultaneously represent the same radical. Alternatively, R³ and R⁴ are different from each other.

W is preferably selected from C₁₋₃ alkylene, C₂₋₃ alkenylene, C₂₋₃ alkynylene, N(R^{b})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NH(CO)—C₀₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy;

W is more preferably selected from C₁₋₃ alkylene, C₁₋₃ alkylene-O—C₁₋₃ alkylene, C₂₋₃ alkenylene, N(R^{b})—C₁₋₂ alkylene, C(O)—C₁₋₂ alkylene, S—C₁₋₂ alkylene, O—C₁₋₂ alkylene, C(O)NH—C₁₋₂ alkylene and NH(CO)—C₁₋₂ alkylene, said alkylene or alkenylene groups or portions of groups optionally being substituted with a group selected from halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy. Preferred halo groups are chloro or fluoro, particularly fluoro. Most preferably, W is selected from C₁₋₃ alkylene, C₁₋₃ alkylene-O—C₁₋₃ alkylene, C(O)—C₁₋₂ alkylene, C(O)NH—C₁₋₂ alkylene and NH(CO)—C₁₋₂ alkylene. Most particularly preferably W is ethylene or C(O)NH—CH₂—. Preferably the alkylene group (for example the ethylene group) is substituted with one or more halo groups, for example one or more fluoro groups (for example one fluoro group). Monohalo C₁₋₃ alkylene (for example fluoro C₁₋₃ alkylene) thus constitutes a preferred group W.

In another preferred embodiment, W is selected from C₁₋₃ alkylene, C₂₋₃ alkenylene, C₁₋₃ alkylene-O—C₁₋₃ alkylene, O—C₁₋₃ alkylene, C(O)NH—C₁₋₂ alkylene and NH(CO)—C₁₋₂ alkylene.

When R¹ is ethyl, W is preferably C₂₋₃ alkenylene, N(R^{b})—C₁₋₂ alkylene, C(O)—C₁₋₂ alkylene, S—C₁₋₂ alkylene, O—C₁₋₂ alkylene, C(O)NH—C₁₋₂ alkylene or NH(CO)—C₁₋₂ alkylene, said alkylene or alkenylene groups or portions of groups optionally being substituted with a group selected from hydroxy, mercapto, amino, halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy.

When R¹ is ethyl and n=0, W is preferably C₂₋₃ alkenylene, N(R^{b})—C₁₋₂ alkylene, C(O)—C₁₋₂ alkylene, S—C₁₋₂ alkylene, O—C₁₋₂ alkylene, C(O)NH—C₁₋₂ alkylene or NH(CO)—C₁₋₂ alkylene, said alkylene, or alkenylene groups or portions of groups optionally being substituted with a group selected from hydroxy, mercapto, amino, halo, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy.

R^{b} is preferably selected from hydrogen, C₁₋₂ alkyl, fluoromethyl, difluoromethyl and trifluoromethyl;

R⁵ is preferably selected from —CO₂R^{c}, —PO(OR^{c})₂, —SO₂OR^{c}, —NHSO₂R^{c′}, —COCO₂R^{c} and CONR^{c}R^{c}. More preferably, R⁵ is —CO₂R^{c}, —PO(OR^{c})₂ or —SO₂OR^{c}. Most preferably, R⁵ is —CO₂R^{c}, particularly —CO₂H.

R^{c} is preferably ethyl, methyl or hydrogen, particularly hydrogen.

R^{c′} is preferably selected from R^{c}, phenyl and phenyl substituted with 1, 2 or 3 groups independently selected from amino, hydroxyl, halogen and methyl.

Accordingly, one preferred group of compounds of the invention includes compounds according to formula (Ia) or pharmaceutically acceptable esters, amides, solvates or salts thereof, including salts of such esters or amides, and solvates of such esters, amides or salts

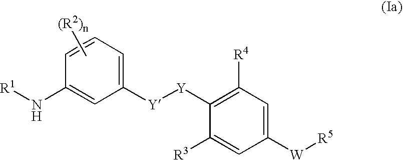

wherein:
n is 0, 1, 2 or 3;
When n=0 and simultaneously R³ and R⁴ are both Br, R¹ is selected from methyl, n-propyl, i-propyl, cyclobutyl, i-butyl n-butyl, t-butyl, C₂₋₄ alkenyl and C₃₋₆ cycloalkyl-C₁₋₃ alkyl, said methyl, propyl, butyl, alkyl or alkenyl groups or portions of groups optionally being substituted with 1, 2 or 3 groups independently selected from halogen, methoxy, halomethoxy, dihalomethoxy, and trihalomethoxy, said cycloalkyl groups or portions of groups optionally being substituted with 1, 2 or 3 groups independently selected from halogen, methyl, ethyl, methoxy, halomethoxy, dihalomethoxy, and trihalomethoxy;
When n=0 and simultaneously R³ and R⁴ are not both Br, or when n=1, 2 or 3, R¹ is selected from hydrogen, C₁₋₄ alkyl, C₂₋₄ alkenyl and C₃₋₆ cycloalkyl-C₁₋₃ alkyl, said alkyl or alkenyl groups or portions of groups optionally being substituted with 1, 2 or 3 groups independently selected from halogen, methoxy, halomethoxy, dihalomethoxy, and trihalomethoxy, said cycloalkyl groups or portions of groups optionally being substituted with 1, 2 or 3 groups independently selected from halogen, methyl, ethyl, methoxy, halomethoxy, dihalomethoxy, and trihalomethoxy;
Each R² is independently selected from halogen, C₁₋₂ alkyl, C₂₋₃ alkenyl, C₂₋₃ alkynyl, C₁₋₂ alkoxy, haloC₁₋₂ alkyl, dihaloC₁₋₂ alkyl, and trihaloC₁₋₂ alkyl;
Y and Y′ together are —C(R^{a′})═C(R^{a′})—,
or alternatively Y is O or S and Y′ is —CH(R^{a})—;
R^{a} is selected from hydrogen, halogen, methyl, ethyl, fluoromethyl, difluoromethyl, trifluoromethyl, fluoroethyl, difluoroethyl and trifluoroethyl;
R³ and R⁴ are independently selected from halogen, C₁₋₄ alkyl, fluoromethyl, difluoromethyl and trifluoromethyl;
W is selected from C₁₋₃ alkylene, C₂₋₃ alkenylene, O—C₁₋₃ alkylene, C₁₋₃ alkylene-O—C₁₋₃ alkylene, C(O)—C₁₋₂ alkylene, C(O)NH—C₁₋₂ alkylene and NH(CO)—C₁₋₂ alkylene; the alkylene group or portion of a group optionally being substituted with one or more halo groups.
R⁵ is selected from —CO₂R^{c}, —PO(OR^{c})₂, —SO₂OR^{c}, —NHSO₂R^{c′}, —COCO₂R^{c} and CONR^{c}OR^{c};
Each R^{c} is independently selected from ethyl, methyl and hydrogen; and
R^{c} is selected from R^{c}, phenyl and phenyl substituted with 1, 2 or 3 groups independently selected from amino, hydroxyl, halogen and methyl.

A further preferred group of compounds of the invention includes compounds according to formula (Ib) or a pharmaceutically acceptable esters, amides, solvates or salts thereof, including salts of such esters or amides, and solvates of such esters, amides or salts,

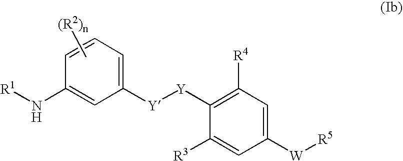

wherein:
n is 0, 1, 2 or 3;
When n=0 and simultaneously R³ and R⁴ are both Br, R¹ is selected from methyl, n-propyl, i-propyl, cyclobutyl, i-butyl n-butyl and t-butyl, C₂₋₄ alkenyl and C₃₋₆ cycloalkyl-C₁₋₃ alkyl;
When n=0 and simultaneously R³ and R⁴ are not both Br, or when n=1, 2 or 3, R¹ is selected from hydrogen, C₁₋₄ alkyl, C₂₋₄ alkenyl and C₃₋₆ cycloalkyl-C₁₋₃ alkyl;
Each R² is independently selected from halogen, C₁₋₂ alkyl, C₂₋₃ alkenyl, C₂₋₃ alkynyl, C₁₋₂ alkoxy, haloC₁₋₂ alkyl, dihaloC₁₋₂ alkyl, and trihaloC₁₋₂ alkyl.
Y and Y′ together are —C(R^{a′})═C(R^{a′})—,
or alternatively Y is O and Y′ is —CH(R^{a})—;
R^{a} is selected from hydrogen, halogen, and C₁₋₂ alkyl;
R^{a′} is selected from hydrogen, halogen, and C₁₋₂ alkyl;
R³ and R⁴ are independently selected from halogen, C₁₋₄ alkyl, fluoromethyl, difluoromethyl and trifluoromethyl;
W is selected from C₁₋₃ alkylene, C₂₋₃ alkenylene, O—C₁₋₃ alkylene, C₁₋₃ alkylene-O—C₁₋₃ alkylene, C(O)NH—C₁₋₂ alkylene and NH(CO)—C₁₋₂ alkylene; the alkylene group or portion of a group optionally being substituted with one or more halo groups.

### R⁵ is —CO₂R^{c};

Each R^{c} is independently selected from ethyl, methyl and hydrogen.

Preferred compounds according to the invention include:
3-(3,5-dibromo-4-{[3-(methylamino)benzyl]oxy}phenyl)propanoic acid
4-[(3-aminobenzyl)oxy]-3,5-dibromobenzoic acid
3-(4-{[3-amino-5-(trifluoromethyl)benzyl]oxy}-3,5-dibromophenyl)propanoic acid
3-(3,5-dibromo-4-{[3-(methylamino)-5-(trifluoromethyl)benzyl]oxy}phenyl)propanoic acid
3-(3,5-dibromo-4-{[2-methyl-3-(methylamino)benzyl]oxy}phenyl)propanoic acid
3-(3,5-dibromo-4-{[3-(ethylamino)-5-(trifluoromethyl)benzyl]oxy}phenyl)propanoic acid
3-{4-[(3-amino-5-chlorobenzyl)oxy]-3,5-dibromophenyl}propanoic acid
3-(3,5-dibromo-4-{[3-chloro-5-(ethylamino)benzyl]oxy}phenyl)propanoic acid
3-{4-[(3-amino-5-methylbenzyl)oxy]-3,5-dibromophenyl}propanoic acid
3-(3,5-dibromo-4-{[3-(ethylamino)-5-methylbenzyl]oxy}phenyl)propanoic acid
3-{4-[(3-amino-4-methylbenzyl)oxy]-3,5-dibromophenyl}propanoic acid
3-(3,5-dibromo-4-{[3-(ethylamino)-4-methylbenzyl]oxy}phenyl)propanoic acid
3-(3,5-dibromo-4-{[3-(ethylamino)-2-methylbenzyl]oxy}phenyl)propanoic acid
3-(3,5-dibromo-4-{[3-(propylamino)benzyl]oxy}phenyl)propanoic acid
3-(3,5-dibromo-4-{[2-methyl-3-(propylamino)benzyl]oxy}phenyl)propanoic acid
3-[3,5-dibromo-4-({3-[(2E)-but-2-en-1-ylamino]benzyl}oxy)phenyl]propanoic acid
3-[3,5-dibromo-4-({3-[(3,3-dimethylbutyl)amino]benzyl}oxy)phenyl]propanoic acid
3-{3,5-dibromo-4-[(3-{[2-methylthio)ethyl]amino}benzyl)oxy]phenyl}propanoic acid
3-(3,5-dibromo-4-{[3-(isopropylamino)benzyl]oxy}phenyl)propanoic acid
3-(3,5-dibromo-4-{[3-(isopropylamino)-5-(trifluoromethyl)benzyl]oxy}phenyl)propanoic acid
3-(3,5-dibromo-4-{[3-(butylamino)benzyl]oxy}phenyl)propanoic acid
3-(3,5-dibromo-4-{[3-(sec-butylamino)benzyl]oxy}phenyl)propanoic acid
3-(3,5-dibromo-4-{[3-(sec-butylamino)-2-methylbenzyl]oxy}phenyl)propanoic acid
3-(3,5-dibromo-4-{[3-[(2-ethylbutyl)amino]benzyl}oxy)phenyl]propanoic acid
3-(3,5-dibromo-4-{[3-(cyclobutylamino)benzyl]oxy}phenyl)propanoic acid
3-(3,5-dibromo-4-{[3-(cyclobutylamino)-2-methylbenzyl]oxy}phenyl)propanoic acid
3-[3,5-dibromo-4-({3-[(cyclopropylmethyl)amino]benzyl}oxy)phenyl]propanoic acid
3-[3,5-dibromo-4-({3-[(cyclohexylmethyl)amino]benzyl}oxy)phenyl]propanoic acid
3-(3,5-dibromo-4-{[3-(isobutylamino)benzyl]oxy}phenyl)propanoic acid
3-{4-[(3-aminobenzyl)oxy]-3,5-dichlorophenyl}propanoic acid
3-(3,5-dichloro-4-{[3-(methylamino)benzyl]oxy}phenyl)propanoic acid
3-(3,5-dichloro-4-{[3-(ethylamino)benzyl]oxy}phenyl)propanoic acid
3-(3,5-dichloro-4-{[3-(ethylamino)-5-(trifluoromethyl)benzyl]oxy}phenyl)propanoic acid
3-(3,5-dichloro-4-{[3-(propylamino)benzyl]oxy}phenyl)propanoic acid
3-(3,5-dichloro-4-{[3-(isopropylamino)benzyl]oxy}phenyl)propanoic acid
3-(4-{[3-(sec-butylamino)benzyl]oxy}-3,5-dichlorophenyl)propanoic acid
3-[3,5-dichloro-4-{3-[(cyclopropylmethyl)amino]benzyl}oxy)phenyl]propanoic acid
N-{4-[(3-aminobenzyl)oxy]-3,5-dibromobenzoyl}glycine
N-(3,5-dibromo-4-{[3-(ethylamino)benzyl]oxy}benzoyl)glycine
N-(3,5-dibromo-4-{[3-(methylamino)benzyl]oxy}benzoyl)glycine
N-(3,5-dibromo-4-{[3-(propylamino)benzyl]oxy}benzoyl)glycine
N-(3,5-dibromo-4-{[3-(isopropylamino)benzyl]oxy}benzoyl)glycine
N-(3,5-dibromo-4-{[3-(cyclobutylamino)benzyl]oxy}benzoyl)glycine
N-(3,5-dibromo-4-{[3-(sec-butylamino)benzyl]oxy}benzoyl)glycine
N-(3,5-dichloro-4-{[3-(ethylamino)benzyl]oxy}benzoyl)glycine
N-(3,5-dibromo-4-{[3-(ethylamino)-5-(trifluoromethyl)benzyl]oxy}benzoyl)glycine
N-(3,5-dibromo-4-{[3-chloro-5-(ethylamino)benzyl]oxy}benzoyl)glycine
N-(3,5-dibromo-4-{[3-(ethylamino)-5-methylbenzyl]oxy}benzoyl)glycine
3-(3,5-dichloro-4-{[3-(ethylamino)-5-methylbenzyl]oxy}phenyl)propanoic acid
3-(3,5-dibromo-4-{[3-chloro-5-(ethylamino)benzyl]oxy}phenyl)-2-fluoropropanoic acid
(3,5-dichloro-4-{[3-(ethylamino)-5-methylbenzyl]oxy}phenyl)acetic acid
(3,5-dibromo-4-{[3-(ethylamino)benzyl]oxy}phenyl)acetic acid
3-(3,5-dibromo-4-{[3-cyano-5-(ethylamino)benzyl]oxy}phenyl)propanoic acid
3-(3,5-dibromo-4-{[3-(ethylamino)-2-fluorobenzyl]oxy}phenyl)propanoic acid
3-(3,5-dibromo-4-{[2-chloro-3-(ethylamino)benzyl]oxy}phenyl)propanoic acid
N-(3,5-dibromo-4-{[2-chloro-3-(ethylamino)benzyl]oxy}benzoyl)glycine
3-(3,5-dibromo-4-{[3-chloro-5-(methylamino)benzyl]oxy}phenyl)propanoic acid
3-(3,5-dibromo-4-{[3-methyl-5-(methylamino)benzyl]oxy}phenyl)propanoic acid
3-(3,5-dibromo-4-{([3-(cyclobutylamino)-5-methylbenzyl]oxy}phenyl)propanoic acid
N-(3,5-dibromo-4-{[3-(cyclobutylamino)-5-methylbenzyl]oxy}benzoyl)glycine
3-(3,5-dichloro-4-{[3-(cyclobutylamino)-5-methylbenzyl]oxy}phenyl)propanoic acid
3-(3,5-dibromo-4-{[3-chloro-5-(cyclobutylamino)benzyl]oxy}phenyl)propanoic acid
N-(3,5-dibromo-4-{[3-chloro-5-(cyclobutylamino)benzyl]oxy}benzoyl)glycine
3-(3,5-dibromo-4-{[3-(cyclobutylamino)-5-(trifluoromethyl)benzyl]oxy}phenyl)propanoic acid
N-(3,5-dibromo-4-{[3-(cyclobutylamino)-5-(trifluoromethyl)benzyl]oxy}benzoyl)glycine
3-(3,5-dibromo-4-{[3-cyano-5-(cyclobutylamino)benzyl]oxy}phenyl)propanoic acid
N-(3,5-dibromo-4-{[2-chloro-3-(cyclobutylamino)benzyl]oxy}benzoyl)glycine
3-(3,5-dibromo-4-{[2-chloro-3-(cyclobutylamino)benzyl]oxy}phenyl)propanoic acid
3-(3,5-dibromo-4-{[3-(isopropylamino)-5-methylbenzyl]oxy}phenyl)propanoic acid
N-(3,5-dibromo-4-{[3-(isopropylamino)-5-methylbenzyl]oxy}benzoyl)glycine
3-(3,5-dichloro-4-{[3-(isopropylamino)-5-methylbenzyl]oxy}phenyl)propanoic acid
3-(3,5-dibromo-4-{[3-chloro-5-(isopropylamino)benzyl]oxy}phenyl)propanoic acid
N-(3,5-dibromo-4-{[3-chloro-5-(isopropylamino)benzyl]oxy}benzoyl)glycine
N-(3,5-dibromo-4-{[3-(isopropylamino)-5-(trifluoromethyl)benzyl]oxy}benzoyl)glycine
3-(3,5-dibromo-4-{[3-chloro-5-(isopropylamino)benzyl]oxy}phenyl)-2-fluoropropanoic acid
N-(3,5-dibromo-4-{[2-chloro-3-(isopropylamino)benzyl]oxy}benzoyl)glycine
3-(3,5-dibromo-4-{[2-chloro-3-(isopropylamino)benzyl]oxy}phenyl)propanoic acid
3-(3,5-dibromo-4-{[3-(sec-butylamino)-5-methylbenzyl]oxy}phenyl)propanoic acid
N-(3,5-dibromo-4-{[3-(sec-butylamino)-5-methylbenzyl]oxy}benzoyl)glycine
3-(4-{[3-(sec-butylamino)-5-methylbenzyl]oxy}-3,5-dichlorophenyl)propanoic acid
3-(3,5-dibromo-4-{[3-(sec-butylamino)-5-chlorobenzyl]oxy}phenyl)propanoic acid
N-(3,5-dibromo-4-{[3-(sec-butylamino)-5-chlorobenzyl]oxy}benzoyl)glycine
3-(3,5-dibromo-4-{[3-(sec-butylamino)-5-(trifluoromethyl)benzyl]oxy}phenyl)propanoic acid
N-(3,5-dibromo-4-{[3-(sec-butylamino)-5-(trifluoromethyl)benzyl]oxy}benzoyl)glycine
3-(3,5-dibromo-4-{[3-(sec-butylamino)-2-chlorobenzyl]oxy}phenyl)propanoic acid
(3,5-dibromo-4-{[3-chloro-5-(ethylamino)benzyl]oxy}phenyl)acetic acid
(3,5-dichloro-4-{[3-(ethylamino)-5-methyl-benzyl]oxy}phenyl)acetic acid
[4-(3-amino-5-methylbenzyloxy)-3,5-dichlorophenyl]acetic acid
N-(3,5-dichloro-4-{[3-(ethylamino)-5-methylbenzyl]oxy}benzoyl)glycine
N-{4-[(3-amino-5-methylbenzyl)oxy]-3,5-dichlorobenzoyl}glycine
3-(3,5-dibromo-4-{[3-(ethylamino)-5-(fluoromethyl)benzyl]oxy}phenyl)propanoic acid
3-(3,5-dibromo-4-{[3-ethoxymethyl-5-(ethylamino)benzyl]oxy}phenyl)propanoic acid
3-(4-[3-amino-2-chlorobenzyloxy]-3,5-bromophenyl) propanoic acid
3-(4-[3-amino-2-fluorobenzyloxy]-3,5-bromophenyl) propanoic acid
3-(3,5-dibromo-4-{[2-ethoxy-3-(ethylamino)benzyl]oxy}phenyl)propanoic acid
3-(3,5-dibromo-4-{[2-methoxy-3-(ethylamino)benzyl]oxy}phenyl)propanoic acid
3-(4-{[3-(2-carboxy-ethylamino)benzyl]oxy}-3,5-dichlorophenyl)propanoic acid
3-[3,5-dichloro-4-({3-[(cyclopropyl)amino]benzyl}oxy)phenyl]propanoic acid
3-(3,5-dibromo-4-{[2-fluoro-5-(isopropylamino)benzyl]oxy}phenyl)propanoic acid
3-(3,5-dibromo-4-{[2-fluoro-5-(cyclobutylamino)benzyl]oxy}phenyl)propanoic acid
3-(3,5-dibromo-4-{[2-fluoro-5-(sec-butylamino)benzyl]oxy}phenyl)propanoic acid
3-(3,5-dibromo-4-{[2-fluoro-5-(ethylamino)benzyl]oxy}phenyl)propanoic acid
3-(3,5-dibromo-4-{[2,5-dichloro-3-(ethylamino)benzyl]oxy}phenyl)propanoic acid
3-(3,5-dibromo-4-{[2,5-dichloro-3-(isopropylamino)benzyl]oxy}phenyl)propanoic acid
3-(3,5-dibromo-4-{[2,5-dichloro-3-(cyclobutylamino)benzyl]oxy}phenyl)propanoic acid
3-(3,5-dibromo-4-{[3-chloro-5-(1,2-dimethyl-propylamino)benzyl]oxy}phenyl)propanoic acid
3-(3,5-dibromo-4-{[2-chloro-3-(1,2-dimethyl-propylamino)benzyl]oxy}phenyl)propanoic acid
N-(3,5-dibromo-4-{[3-chloro-5-(1,2-dimethyl-propylamino)benzyl]oxy}benzoyl)glycine
3-(3,5-dibromo-4-{[2,5-dichloro-3-(isopropylamino)benzyl]oxy}phenyl)-2-fluoropropanoic acid
3-(3,5-dibromo-4-{[2,5-dichloro-3-(ethylamino)benzyl]oxy}phenyl)-2-fluoropropanoic acid
3-(3,5-dibromo-4-{[2-chloro-3-(ethylamino)benzyl]oxy}phenyl)-2-fluoropropanoic acid
3-(3,5-dibromo-4-{[3-(ethylamino)-5-methylbenzyl]oxy}phenyl)butanoic acid
3-(3,5-dibromo-4-{[3-(isopropylamino)-5-methylbenzyl]oxy}phenyl)butanoic acid
3-(3,5-dibromo-4-{[3-(cyclobutylamino)-5-methylbenzyl]oxy}phenyl)butanoic acid
3-(3,5-dibromo-4-{[2,5-dichloro-3-(ethylamino)benzyl]oxy}phenyl)butanoic acid
(E)-3-(3,5-dibromo-4-{[2,5-dichloro-3-(ethylamino)benzyl]oxy}phenyl)acrylic acid
(E)-3-(3,5-dibromo-4-{[2,5-dichloro-3-(isopropylamino)benzyl]oxy}phenyl)acrylic acid
(E)-3-(3,5-dibromo-4-{[2,5-dichloro-3-(cyclobutylamino)benzyl]oxy}phenyl)acrylic acid
N-(3,5-dibromo-4-{[2,5-dichloro-3-(ethylamino)benzyl]oxy}benzoyl)glycine
N-3,5-dibromo-4-{[2,5-dichloro-3-(isopropylamino)benzyl]oxy}benzoyl)glycine
N-(3,5-dibromo-4-{[2,5-dichloro-3-(cyclobutylamino)benzyl]oxy}benzoyl)glycine
(S)-2-{2-(3,5-dibromo-4-{[3-chloro-5-(ethylamino)benzyl]oxy}phenyl)acetylamino}-3-phenyl-propanoic acid
(S)-2-{2-(3,5-dichloro-4-{[3-(ethylamino)-5-methylbenzyl]oxy}phenyl)acetylamino}-2-phenyl-acetic acid
3-[(3,5-dibromo-4-{[3-chloro-5-(isopropylamino)benzyl]oxy}phenyl)amino]-3-oxopropanoic acid
3-(3,5-dichloro-4-{[3-chloro-5-(ethylamino)benzyl]oxy}phenyl)propanoic acid
3-(3,5-dichloro-4-{[3-(ethylamino)-5-(trifluoromethyl)benzyl]oxy}phenyl)-2-fluoropropanoic acid
3-(3,5-dichloro-4-{[3-chloro-5-(ethylamino)benzyl]oxy}phenyl)-2-fluoropropanoic acid
3-(3,5-dichloro-4-{[3-(ethylamino)-5-methylbenzyl]oxy}phenyl)-2-fluoropropanoic acid
3-(3,5-dibromo-4-{[3-(ethylamino)-5-(trifluoromethyl)benzyl]oxy}phenyl)-2-fluoropropanoic acid
3-(3,5-dibromo-4-{[3-(ethylamino)-5-methylbenzyl]oxy}phenyl)-2-fluoropropanoic acid
(3,5-dichloro-4-{[3-(ethylamino)-5-(trifluoromethyl)benzyl]oxy}phenyl)acetic acid
N-(3,5-dichloro-4-{[3-(ethylamino)-5-(trifluoromethyl)benzyl]oxy}benzoyl)glycine
(3,5-dibromo-4-{[3-(ethylamino)-5-(trifluoromethyl)benzyl]oxy}phenyl)acetic acid
(3,5-dichloro-4-{[3-chloro-5-(ethylamino)benzyl]oxy}phenyl)acetic acid
N-(3,5-dichloro-4-{[3-chloro-5-(ethylamino)benzyl]oxy}benzoyl)glycine
(3,5-dibromo-4-{[3-(ethylamino)-5-methylbenzyl]oxy}phenyl)acetic acid
3-[(3,5-dichloro-4-{[3-(ethylamino)-5-(trifluoromethyl)benzyl]oxy}phenyl)amino]-3-oxopropanoic acid
3-[(3,5-dibromo-4-{[3-(ethylamino)-5-(trifluoromethyl)benzyl]oxy}phenyl)amino]-3-oxopropanoic acid
3-[(4-{[3-(ethylamino)-5-(trifluoromethyl)benzyl]oxy}-3,5-dimethylphenyl)amino]-3-oxopropanoic acid
3-[(3,5-dichloro-4-{[3-chloro-5-(ethylamino)benzyl]oxy}phenyl)amino]-3-oxopropanoic acid
3-[(3,5-dibromo-4-{[3-chloro-5-(ethylamino)benzyl]oxy}phenyl)amino]-3-oxopropanoic acid
3-[(4-{[3-chloro-5-(ethylamino)benzyl]oxy}-3,5-dimethylphenyl)amino]-3-oxopropanoic acid
3-[(3,5-dichloro-4-{[3-(ethylamino)-5-methylbenzyl]oxy}phenyl)amino]-3-oxopropanoic acid
3-[(3,5-dibromo-4-{[3-(ethylamino)-5-methylbenzyl]oxy}phenyl)amino]-3-oxopropanoic acid
3-[(4-{[3-(ethylamino)-5-methylbenzyl]oxy}-3,5-dimethylphenyl)amino]-3-oxopropanoic acid
(3,5-dichloro-4-{[3-(ethylamino)benzyl]oxy}phenyl)acetic acid
3-(3,5-dichloro-4-{[3-(ethylamino)-5-fluorobenzyl]oxy}phenyl)propanoic acid
N-(3,5-dibromo-4-{[3-(ethylamino)-5-fluorobenzyl]oxy}benzoyl)glycine
3-(3,5-dibromo-4-{[3-(ethylamino)-5-fluorobenzyl]oxy}phenyl)propanoic acid
(3,5-dichloro-4-{[3-(ethylamino)-5-fluorobenzyl]oxy}phenyl)acetic acid
N-(3,5-dichloro-4-{[3-(ethylamino)-5-fluorobenzyl]oxy}benzoyl)glycine
(3,5-dibromo-4-{[3-(ethylamino)-5-fluorobenzyl]oxy}phenyl)acetic acid
3-(3,5-dichloro-4-{[3-cyano-5-(ethylamino)benzyl]oxy}phenyl)propanoic acid
N-(3,5-dibromo-4-{[3-cyano-5-(ethylamino)benzyl]oxy}benzoyl)glycine
(3,5-dichloro-4-{[3-cyano-5-(ethylamino)benzyl]oxy}phenyl)acetic acid
N-(3,5-dichloro-4-{[3-cyano-5-(ethylamino)benzyl]oxy}benzoyl)glycine
(3,5-dibromo-4-{[3-cyano-5-(ethylamino)benzyl]oxy}phenyl)acetic acid
3-(3,5-dichloro-4-{[3-(ethylamino)-2-fluorobenzyl]oxy}phenyl)propanoic acid
N-(3,5-dibromo-4-{[3-(ethylamino)-2-fluorobenzyl]oxy}benzoyl)glycine
(3,5-dichloro-4-{[3-(ethylamino)-2-fluorobenzyl]oxy}phenyl)acetic acid
N-(3,5-dichloro-4-{[3-(ethylamino)-2-fluorobenzyl]oxy}benzoyl)glycine
(3,5-dibromo-4-{[3-(ethylamino)-2-fluorobenzyl]oxy}phenyl)acetic acid
3-(3,5-dichloro-4-{[2-chloro-3-(ethylamino)benzyl]oxy}phenyl)propanoic acid
(3,5-dibromo-4-{[2-chloro-3-(ethylamino)benzyl]oxy}phenyl)acetic acid
N-(3,5-dichloro-4-{[2-chloro-3-(ethylamino)benzyl]oxy}benzoyl)glycine
(3,5-dichloro-4-{[2-chloro-3-(ethylamino)benzyl]oxy}phenyl)acetic acid
3-(3,5-dichloro-4-{[3-(ethylamino)-2-fluoro-5-methylbenzyl]oxy}phenyl)propanoic acid
N-(3,5-dibromo-4-{[3-(ethylamino)-2-fluoro-5-methylbenzyl]oxy}benzoyl)glycine
3-(3,5-dibromo-4-{[3-(ethylamino)-2-fluoro-5-methylbenzyl]oxy}phenyl)propanoic acid
(3,5-dichloro-4-{[3-(ethylamino)-2-fluoro-5-methylbenzyl]oxy}phenyl)acetic acid
N-(3,5-dichloro-4-{[3-(ethlylamino)-2-fluoro-5-methylbenzyl]oxy}benzoyl)glycine
(3,5-dibromo-4-{[3-(ethylamino)-2-fluoro-5-methylbenzyl]oxy}phenyl)acetic acid
3-(3,5-dichloro-4-{[5-chloro-3-(ethylamino)-2-fluorobenzyl]oxy}phenyl)propanoic acid
N-(3,5-dibromo-4-{[5-chloro-3-(ethylamino)-2-fluorobenzyl]oxy}benzoyl)glycine
3-(3,5-dibromo-4-{[5-chloro-3-(ethylamino)-2-fluorobenzyl]oxy}phenyl)propanoic acid
(3,5-dichloro-4-{[5-chloro-3-(ethylamino)-2-fluorobenzyl]oxy}phenyl)acetic acid
N-(3,5-dichloro-4-{[5-chloro-3-(ethylamino)-2-fluorobenzyl]oxy benzoyl)glycine
(3,5-dibromo-4-([5-chloro-3-(ethylamino)-2-fluorobenzyl]oxy}phenyl)acetic acid
3-(3,5-dichloro-4-{[3-(methylamino)-5-(trifluoromethyl)benzyl]oxy}phenyl)propanoic acid
N-(3,5-dibromo-4-{[3-(methylamino)-5-(trifluoromethyl)benzyl]oxy}benzoyl)glycine
3-(3,5-dichloro-4-{[3-chloro-5-(methylamino)benzyl]oxy}phenyl)propanoic acid
N-(3,5-dibromo-4-{[3-chloro-5-(methylamino)benzyl]oxy}benzoyl)glycine
3-(3,5-dichloro-4-{[3-methyl-5-(methylamino)benzyl]oxy}phenyl)propanoic acid
N-(3,5-dibromo-4-{[3-methyl-5-(methylamino)benzyl]oxy}benzoyl)glycine
3-(3,5-dichloro-4-{[3-(methylamino)-5-(trifluoromethyl)benzyl]oxy}phenyl)-2-fluoropropanoic acid
3-(3,5-dichloro-4-{[3-chloro-5-(methylamino)benzyl]oxy}phenyl)-2-fluoropropanoic acid
3-(3,5-dichloro-4-{[3-methyl-5-(methylamino)benzyl]oxy}phenyl)-2-fluoropropanoic acid
3-(3,5-dibromo-4-{[3-(methylamino)-5-(trifluoromethyl)benzyl]oxy}phenyl)-2-fluoropropanoic acid
3-(3,5-dibromo-4-{[3-chloro-5-(methylamino)benzyl]oxy}phenyl)-2-fluoropropanoic acid
3-(3,5-dibromo-4-{[3-methyl-5-(methylamino)benzyl]oxy}phenyl)-2-fluoropropanoic acid
(3,5-dichloro-4-{[3-(methylamino)-5-(trifluoromethyl)benzyl]oxy}phenyl)acetic acid
N-(3,5-dichloro-4-{[3-(methylamino)-5-(trifluoromethyl)benzyl]oxy}benzoyl)glycine
(3,5-dibromo-4-{[3-(methylamino)-5-(trifluoromethyl)benzyl]oxy}phenyl)acetic acid
(3,5-dichloro-4-{[3-chloro-5-(methylamino)benzyl]oxy}phenyl)acetic acid
N-(3,5-dichloro-4-{[3-chloro-5-(methylamino)benzyl]oxy}benzoyl)glycine
(3,5-dibromo-4-{[3-chloro-5-(methylamino)benzyl]oxy}phenyl)acetic acid
(3,5-dichloro-4-{[3-methyl-5-(methylamino)benzyl]oxy}phenyl)acetic acid
N-(3,5-dichloro-4-{[3-methyl-5-(methylamino)benzyl]oxy}benzoyl)glycine
(3,5-dibromo-4-{[3-methyl-5-(methylamino)benzyl]oxy}phenyl)acetic acid
3-[(3,5-dichloro-4-{[3-(methylamino)-5-(trifluoromethyl)benzyl]oxy}phenyl)amino]-3-oxopropanoic acid
3-[(3,5-dibromo-4-{[3-(methylamino)-5-(trifluoromethyl)benzyl]oxy}phenyl)amino]-3-oxopropanoic acid
3-[(3,5-dimethyl-4-{[3-(methylamino)-5-(trifluoromethyl)benzyl]oxy}phenyl)amino]-3-oxopropanoic acid
3-[(3,5-dichloro-4-{[3-chloro-5-(methylamino)benzyl]oxy}phenyl)amino]-3-oxopropanoic acid
3-[(3,5-dibromo-4-{[3-chloro-5-(methylamino)benzyl]oxy}phenyl)amino]-3-oxopropanoic acid
3-[(4-{[3-chloro-5-(methylamino)benzyl]oxy}-3,5-dimethylphenyl)amino]-3-oxopropanoic acid
3-[(3,5-dichloro-4-{[3-methyl-5-(methylamino)benzyl]oxy}phenyl)amino]-3-oxopropanoic acid
3-[(3,5-dibromo-4-{[3-methyl-5-(methylamino)benzyl]oxy}phenyl)amino]-3-oxopropanoic acid
3-[(3,5-dimethyl-4-{[3-methyl-5-(methylamino)benzyl]oxy}phenyl)amino]-3-oxopropanoic acid
(3,5-dichloro-4-{[3-(methylamino)benzyl]oxy}phenyl)acetic acid
N-(3,5-dichloro-4-{[3-(methylamino)benzyl]oxy}benzoyl)glycine
(3,5-dibromo-4-{[3-(methylamino)benzyl]oxy}phenyl)acetic acid
3-(3,5-dichloro-4-{[3-fluoro-5-(methylamino)benzyl]oxy}phenyl)propanoic acid
N-(3,5-dibromo-4-{[3-fluoro-5-(methylamino)benzyl]oxy}benzoyl)glycine
3-(3,5-dibromo-4-{[3-fluoro-5-(methylamino)benzyl]oxy}phenyl)propanoic acid
(3,5-dichloro-4-{[3-fluoro-5-(methylamino)benzyl]oxy}phenyl)acetic acid
N-(3,5-dichloro-4-{[3-fluoro-5-(methylamino)benzyl]oxy}benzoyl)glycine
(3,5-dibromo-{[3-fluoro-5-(methylamino)benzyl]oxy}phenyl)acetic acid
3-(3,5-dichloro-4-{[3-cyano-5-(methylamino)benzyl]oxy}phenyl)propanoic acid
N-(3,5-dibromo-4-{[3-cyano-5-(methylamino)benzyl]oxy}benzoyl)glycine
3-(3,5-dibromo-4-{[3-cyano-5-(methylamino)benzyl]oxy}phenyl)propanoic acid
(3,5-dichloro-4-{[3-cyano-5-(methylamino)benzyl]oxy}phenyl)acetic acid
N-(3,5-dichloro-4-{[3-cyano-5-(methylamino)benzyl]oxy}benzoyl)glycine
(3,5-dibromo-4-{[3-cyano-5-(methylamino)benzyl]oxy}phenyl)acetic acid
3-(3,5-dichloro-4-{[2-fluoro-3-(methylamino)benzyl]oxy}phenyl)propanoic acid
N-(3,5-dibromo-4-{[2-fluoro-3-(methylamino)benzyl]oxy}benzoyl)glycine
3-(3,5-dibromo-4-{[2-fluoro-3-(methylamino)benzyl]oxy}phenyl)propanoic acid
(3,5-dichloro-4-{[2-fluoro-3-(methylamino)benzyl]oxy}phenyl)acetic acid
N-(3,5-dichloro-4-{[2-fluoro-3-(methylamino)benzyl]oxy}benzoyl)glycine
(3,5-dibromo-4-{[2-fluoro-3-(methylamino)benzyl]oxy}phenyl)acetic acid
3-(3,5-dichloro-4-{[2-chloro-3-(methylamino)benzyl]oxy}phenyl)propanoic acid
N-(3,5-dibromo-4-{[2-chloro-3-(methylamino)benzyl]oxy}benzoyl)glycine
3-(3,5-dibromo-4-{[2-chloro-3-(methylamino)benzyl]oxy}phenyl)propanoic acid
(3,5-dibromo-4-{[2-chloro-3-(methylamino)benzyl]oxy}phenyl)acetic acid
N-(3,5-dichloro-4-{[2-chloro-3-(methylamino)benzyl]oxy}benzoyl)glycine
(3,5-dichloro-4-{[2-chloro-3-(methylamino)benzyl]oxy}phenyl)acetic acid
3-(3,5-dichloro-4-{[2-fluoro-5-methyl-3-(methylamino)benzyl]oxy}phenyl)propanoic acid
N-(3,5-dibromo-4-{[2-fluoro-5-methyl-3-(methylamino)benzyl]oxy}benzoyl)glycine
3-(3,5-dibromo-4-{[2-fluoro-5-methyl-3-(methylamino)benzyl]oxy}phenyl)propanoic acid
(3,5-dichloro-4-{[2-fluoro-5-methyl-3-(methylamino)benzyl]oxy}phenyl)acetic acid
N-(3,5-dichloro-4-{[2-fluoro-5-methyl-3-(methylamino)benzyl]oxy}benzoyl)glycine
(3,5-dibromo-4-{[2-fluoro-5-methyl-3-(methylamino)benzyl]oxy}phenyl)acetic acid
3-(3,5-dichloro-4-{[5-chloro-2-fluoro-3-(methylamino)benzyl]oxy}phenyl)propanoic acid
N-(3,5-dibromo-4-{[5-chloro-2-fluoro-3-(methylamino)benzyl]oxy}benzoyl)glycine
3-(3,5-dibromo-4-{[5-chloro-2-fluoro-3-(methylamino)benzyl]oxy}phenyl)propanoic acid
(3,5-dichloro-4-{[5-chloro-2-fluoro-3-(methylamino)benzyl]oxy}phenyl)acetic acid
N-(3,5-dichloro-4-{[5-chloro-2-fluoro-3-(methylamino)benzyl]oxy}benzoyl)glycine
(3,5-dibromo-4-{[5-chloro-2-fluoro-3-(methylamino)benzyl]oxy}phenyl)acetic acid
3-(3,5-dichloro-4-{[3-[(cyclopropylmethyl)amino]-5-(trifluoromethyl)benzyl]oxy}phenyl)propanoic acid
N-(3,5-dibromo-4-{[3-[(cyclopropylmethyl)amino]-5-(trifluoromethyl)benzyl]oxy}benzoyl)glycine
3-(3,5-dibromo-4-{[3-[(cyclopropylmethyl)amino]-5-(trifluoromethyl)benzyl]oxy}phenyl)propanoic acid
3-[3,5-dichloro-4-({3-chloro-5-[(cyclopropylmethyl)amino]benzyl}oxy)phenyl]propanoic acid
N-[3,5-dibromo-4-({3-chloro-5-[(cyclopropylmethyl)amino]benzyl}oxy)benzoyl]glycine
3-[3,5-dibromo-4-({3-chloro-5-[(cyclopropylmethyl)amino]benzyl}oxy)phenyl]propanoic acid
3-[3,5-dichloro-4-{3-[(cyclopropylmethyl)amino]-5-methylbenzyl}oxy)phenyl]propanoic acid
N-[3,5-dibromo-4-({3-[(cyclopropylmethyl)amino]-5-methylbenzyl}oxy)benzoyl]glycine
3-[3,5-dibromo-4-({3-[(cyclopropylmethyl)amino]-5-methylbenzyl}oxy)phenyl]propanoic acid
3-(3,5-dichloro-4-{[3-[(cyclopropylmethyl)amino]-5-(trifluoromethyl)benzyl]oxy}phenyl)-2-fluoropropanoic acid
3-[3,5-dichloro-4-({3-chloro-5-[(cyclopropylmethyl)amino]benzyl}oxy)phenyl]-2-fluoropropanoic acid
3-[3,5-dichloro-4-({3-[(cyclopropylmethyl)amino]-5-methylbenzyl}oxy)phenyl]-2-fluoropropanoic acid
3-(3,5-dibromo-4-{[3-[(cyclopropylmethyl)amino]-5-(trifluoromethyl)benzyl]oxy}phenyl)-2-fluoropropanoic acid
3-[3,5-dibromo-4-({3-chloro-5-[(cyclopropylmethyl)amino]benzyl}oxy)phenyl]-2-fluoropropanoic acid
3-[3,5-dibromo-4-({3-[(cyclopropylmethyl)amino]-5-methylbenzyl}oxy)phenyl]-2-fluoropropanoic acid
(3,5-dichloro-4-{([3-[(cyclopropylmethyl)amino]-5-(trifluoromethyl)benzyl]oxy}phenyl)acetic acid
N-(3,5-dichloro-4-{[3-[(cyclopropylmethyl)amino]-5-(trifluoromethyl)benzyl]oxy}benzoyl)glycine
(3,5-dibromo-4-{[3-[(cyclopropylmethyl)amino]-5-(trifluoromethyl)benzyl]oxy}phenyl)acetic acid
[3,5-dichloro-4-({3-chloro-5-[(cyclopropylmethyl)amino]benzyl}oxy)phenyl]acetic acid
N-[3,5-dichloro-4-({3-chloro-5-[(cyclopropylmethyl)amino]benzyl}oxy)benzoyl]glycine
[3,5-dibromo-4-({3-chloro-5-[(cyclopropylmethyl)amino]benzyl}oxy)phenyl]acetic acid
[3,5-dichloro-4-({3-[(cyclopropylmethyl)amino]-5-methylbenzyl}oxy)phenyl]acetic acid
N-[3,5-dichloro-4-({3-[(cyclopropylmethyl)amino]-5-methylbenzyl}oxy)benzoyl]glycine
[3,5-dibromo-4-({3-[(cyclopropylmethyl)amino]-5-methylbenzyl}oxy)phenyl]acetic acid
3-[(3,5-dichloro-4-{[3-[(cyclopropylmethyl)amino]-5-(trifluoromethyl)benzyl]oxy}phenyl)amino]-3-oxopropanoic acid
3-[(3,5-dibromo-4-{[3-[(cyclopropylmethyl)amino]-5-(trifluoromethyl)benzyl]oxy}phenyl)amino]-3-oxopropanoic acid
3-[(4-{[3-[(cyclopropylmethyl)amino]-5-(trifluoromethyl)benzyl]oxy}-3,5-dimethylphenyl)amino]-3-oxopropanoic acid
3-{[3,5-dichloro-4-({3-chloro-5-[(cyclopropylmethyl)amino]benzyl}oxy)phenyl]amino}-3-oxopropanoic acid
3-{[3,5-dibromo-4-({3-chloro-5-[(cyclopropylmethyl)amino]benzyl}oxy)phenyl]amino}-3-oxopropanoic acid
3-{[4-({3-chloro-5-[(cyclopropylmethyl)amino]benzyl}oxy)-3,5-dimethylphenyl]amino}-3-oxopropanoic acid
3-{[3,5-dichloro-4-({3-[(cyclopropylmethyl)amino]-5-methylbenzyl}oxy)phenyl]amino}-3-oxopropanoic acid
3-{[3,5-dibromo-4-({3-[(cyclopropylmethyl)amino]-5-methylbenzyl}oxy)phenyl]amino}-3-oxopropanoic acid
3-{[4-({3-[(cyclopropylmethyl)amino]-5-methylbenzyl}oxy)-3,5-dimethylphenyl]amino}-3-oxopropanoic acid
[3,5-dichloro-4-({3-[(cyclopropylmethyl)amino]benzyl}oxy)phenyl]acetic acid
N-[3,5-dichloro-4-({3-[(cyclopropylmethyl)amino]benzyl}oxy)benzoyl]glycine
[3,5-dibromo-4-({3-[(cyclopropylmethyl)amino]benzyl}oxy)phenyl]acetic acid
3-[3,5-dichloro-4-({3-[(cyclopropylmethyl)amino]-5-fluorobenzyl}oxy)phenyl]propanoic acid
N-[3,5-dibromo-4-({3-[(cyclopropylmethyl)amino]-5-fluorobenzyl}oxy)benzoyl]glycine
3-[3,5-dibromo-4-({3-[(cyclopropylmethyl)amino]-5-fluorobenzyl}oxy)phenyl]propanoic acid
[3,5-dichloro-4-({3-[(cyclopropylmethyl)amino]-5-fluorobenzyl}oxy)phenyl]acetic acid
N-[3,5-dichloro-4-({3-[(cyclopropylmethyl)amino]-5-fluorobenzyl}oxy)benzoyl]glycine
[3,5-dibromo-4-({3-[(cyclopropylmethyl)amino]-5-fluorobenzyl}oxy)phenyl]acetic acid
3-[3,5-dichloro-4-({3-cyano-5-[(cyclopropylmethyl)amino]benzyl}oxy)phenyl]propanoic acid
N-[3,5-dibromo-4-({3-cyano-5-[(cyclopropylmethyl)amino]benzyl}oxy)benzoyl]glycine
3-[3,5-dibromo-4-({3-cyano-5-[(cyclopropylmethyl)amino]benzyl}oxy)phenyl]propanoic acid
[3,5-dichloro-4-({3-cyano-5-[(cyclopropylmethyl)amino]benzyl}oxy)phenyl]acetic acid
N-[3,5-dichloro-4-({3-cyano-5-[(cyclopropylmethyl)amino]benzyl}oxy)benzoyl]glycine
[3,5-dibromo-4-({3-cyano-5-[(cyclopropylmethyl)amino]benzyl}oxy)phenyl]acetic acid
3-[3,5-dichloro-4-({3-[(cyclopropylmethyl)amino]-2-fluorobenzyl}oxy)phenyl]propanoic acid
N-[3,5-dibromo-4-({3-[(cyclopropylmethyl)amino]-2-fluorobenzyloxy}benzoyl]glycine
3-[3,5-dibromo-4-({3-[(cyclopropylmethyl)amino]-2-fluorobenzyl}oxy)phenyl]propanoic acid
[3,5-dichloro-4-({3-[(cyclopropylmethyl)amino]-2-fluorobenzyl}oxy)phenyl]acetic acid
N-[3,5-dichloro-4-({3-[(cyclopropylmethyl)amino]-2-fluorobenzyl}oxy)benzoyl]glycine
[3,5-dibromo-4-({3-[(cyclopropylmethyl)amino]-2-fluorobenzyl}oxy)phenyl]acetic acid
3-[3,5-dichloro-4-({2-chloro-3-[(cyclopropylmethyl)amino]benzyl}oxy)phenyl]propanoic acid
N-[3,5-dibromo-4-({2-chloro-3-[(cyclopropylmethyl)amino]benzyl}oxy)benzoyl]glycine
3-[3,5-dibromo-4-({2-chloro-3-[(cyclopropylmethyl)amino]benzyl}oxy)phenyl]propanoic acid
[3,5-dibromo-4-({2-chloro-3-[(cyclopropylmethyl)amino]benzyl}oxy)phenyl]acetic acid
N-[3,5-dichloro-4-({2-chloro-3-[(cyclopropylmethyl)amino]benzyl}oxy)benzoyl]glycine
[3,5-dichloro-4-({2-chloro-3-[(cyclopropylmethyl)amino]benzyl}oxy)phenyl]acetic acid
3-[3,5-dichloro-4-({3-[(cyclopropylmethyl)amino]-2-fluoro-5-methylbenzyl}oxy)phenyl]propanoic acid
N-[3,5-dibromo-4-({3-[(cyclopropylmethyl)amino]-2-fluoro-5-methylbenzyl}oxy)benzoyl]glycine
3-[3,5-dibromo-4-({3-[(cyclopropylmethyl)amino]-2-fluoro-5-methylbenzyl}oxy)phenyl]propanoic acid
[3,5-dichloro-4-({3-[(cyclopropylmethyl)amino]-2-fluoro-5-methylbenzyl}oxy)phenyl]acetic acid
N-[3,5-dichloro-4-({3-[(cyclopropylmethyl)amino]-2-fluoro-5-methylbenzyl}oxy)benzoyl]glycine
[3,5-dibromo-4-({3-[(cyclopropylmethyl)amino]-2-fluoro-5-methylbenzyl}oxy)phenyl]acetic acid
3-[3,5-dichloro-4-({5-chloro-3-[(cyclopropylmethyl)amino]-2-fluorobenzyl}oxy)phenyl]propanoic acid
N-[3,5-dibromo-4-({5-chloro-3-[(cyclopropylmethyl)amino]-2-fluorobenzyl}oxy)benzoyl]glycine
3-[3,5-dibromo-4-({-chloro-3-[(cyclopropylmethyl)amino]-2-fluorobenzyl}oxy)phenyl]propanoic acid
[3,5-dichloro-4-({5-chloro-3-[(cyclopropylmethyl)amino]-2-fluorobenzyl}oxy)phenyl]acetic acid
N-[3,5-dichloro-4-({5-chloro-3-[(cyclopropylmethyl)amino]-2-fluorobenzyl}oxy)benzoyl]glycine
[3,5-dibromo-4-({5-chloro-3-[(cyclopropylmethyl)amino]-2-fluorobenzyl}oxy)phenyl]acetic acid
3-(3,5-dichloro-4-{[3-(cyclobutylamino)-5-(trifluoromethyl)benzyl]oxy}phenyl)propanoic acid
3-(3,5-dichloro-4-{[3-chloro-5-(cyclobutylamino)benzyl]oxy}phenyl)propanoic acid
3-(3,5-dichloro-4-{[3-(cyclobutylamino)-5-(trifluoromethyl)benzyl]oxy}phenyl)-2-fluoropropanoic acid
3-(3,5-dichloro-4-{[3-chloro-5-(cyclobutylamino)benzyl]oxy}phenyl)-2-fluoropropanoic acid
3-(3,5-dichloro-4-{[3-(cyclobutylamino)-5-methylbenzyl]oxy}phenyl)-2-fluoropropanoic acid
3-(3,5-dibromo-4-{[3-(cyclobutylamino)-5-(trifluoromethyl)benzyl]oxy}phenyl)-2-fluoropropanoic acid
3-(3,5-dibromo-4-{[3-chloro-5-(cyclobutylamino)benzyl]oxy}phenyl)-2-fluoropropanoic acid
3-(3,5-dibromo-4-{[3-(cyclobutylamino)-5-methylbenzyl]oxy}phenyl)-2-fluoropropanoic acid
(3,5-dichloro-4-([3-(cyclobutylamino)-5-(trifluoromethyl)benzyl]oxy)phenyl)acetic acid
N-(3,5-dichloro-4-{[3-(cyclobutylamino)-5-(trifluoromethyl)benzyl]oxy}benzoyl)glycine
(3,5-dibromo-4-{[3-(cyclobutylamino)-5-(trifluoromethyl)benzyl]oxy}phenyl)acetic acid
(3,5-dichloro-4-({[3-chloro-5-(cyclobutylamino)benzyl]oxy}phenyl)acetic acid
N-(3,5-dichloro-4-{[3-chloro-5-(cyclobutylamino)benzyl]oxy)benzoyl)glycine
(3,5-dibromo-4-([3-chloro-5-(cyclobutylamino)benzyl]oxy}phenyl)acetic acid
(3,5-dichloro-4-{[3-(cyclobutylamino)-5-methylbenzyl]oxy}phenyl)acetic acid
N-(3,5-dichloro-4-{[3-(cyclobutylamino)-5-methylbenzyl]oxy}benzoyl)glycine
(3,5-dibromo-4-{[3-(cyclobutylamino)-5-methylbenzyl]oxy}phenyl)acetic acid
3-[(3,5-dichloro-4-{[3-(cyclobutylamino)-5-(trifluoromethyl)benzyl]oxy}phenyl)amino]-3-oxopropanoic acid
3-[(3,5-dibromo-4-{[3-(cyclobutylamino)-5-(trifluoromethyl)benzyl]oxy}phenyl)amino]-3-oxopropanoic acid
3-[(4-{[3-(cyclobutylamino)-5-(trifluoromethyl)benzyl]oxy}-3,5-dimethylphenyl)amino]-3-oxopropanoic acid
3-[(3,5-dichloro-4-{[3-chloro-5-(cyclobutylamino)benzyl]oxy}phenyl)amino]-3-oxopropanoic acid
3-[(3,5-dibromo-4-{[3-chloro-5-(cyclobutylamino)benzyl]oxy}phenyl)amino]-3-oxopropanoic acid
3-[(4-{[3-chloro-5-(cyclobutylamino)benzyl]oxy}-3,5-dimethylphenyl)amino]-3-oxopropanoic acid
3-[(3,5-dichloro-4-{([3-(cyclobutylamino)-5-methylbenzyl]oxy}phenyl)amino]-3-oxopropanoic acid
3-[(3,5-dibromo-4-{[3-(cyclobutylamino)-5-methylbenzyl]oxy}phenyl)amino]-3-oxopropanoic acid
3-[(4-{[3-(cyclobutylamino)-5-methylbenzyl]oxy}-3,5-dimethylphenyl)amino]-3-oxopropanoic acid
(3,5-dichloro-4-{[3-(cyclobutylamino)benzyl]oxy}phenyl)acetic acid
N-(3,5-dichloro-4-{[3-(cyclobutylamino)benzyl]oxy}benzoyl)glycine
(3,5-dibromo-4-{[3-(cyclobutylamino)benzyl]oxy}phenyl)acetic acid
3-(3,5-dichloro-4-{[3-(cyclobutylamino)-5-fluorobenzyl]oxy}phenyl)propanoic acid
N-(3,5-dibromo-4-{[3-(cyclobutylamino)-5-fluorobenzyl]oxy}benzoyl)glycine
3-(3,5-dibromo-4-{[3-(cyclobutylamino)-5-fluorobenzyl]oxy}phenyl)propanoic acid
(3,5-dichloro-4-{[3-(cyclobutylamino)-5-fluorobenzyl]oxy}phenyl)acetic acid
N-(3,5-dichloro-4-{[3-(cyclobutylamino)-5-fluorobenzyl]oxy}benzoyl)glycine
(3,5-dibromo-4-{[3-(cyclobutylamino)-5-fluorobenzyl]oxy}phenyl)acetic acid
3-(3,5-dichloro-4-{[3-cyano-5-(cyclobutylamino)benzyl]oxy}phenyl)propanoic acid
N-(3,5-dibromo-4-{[3-cyano-5-(cyclobutylamino)benzyl]oxy)benzoyl)glycine
(3,5-dichloro-4-([3-cyano-5-(cyclobutylamino)benzyl]oxy}phenyl)acetic acid
N-(3,5-dichloro-4-{[3-cyano-5-(cyclobutylamino)benzyl]oxy}benzoyl)glycine
(3,5-dibromo-4-{[3-cyano-5-(cyclobutylamino)benzyl]oxy}phenyl)acetic acid
3-(3,5-dichloro-4-{[3-(cyclobutylamino)-2-fluorobenzyl]oxy}phenyl)propanoic acid
N-(3,5-dibromo-4-{[3-(cyclobutylamino)-2-fluorobenzyl]oxy}benzoyl)glycine
3-(3,5-dibromo-4-{[3-(cyclobutylamino)-2-fluorobenzyl]oxy}phenyl)propanoic acid
(3,5-dichloro-4-{[3-(cyclobutylamino)-2-fluorobenzyl]oxy}phenyl)acetic acid
N-(3,5-dichloro-4-([3-(cyclobutylamino)-2-fluorobenzyl]oxy)benzoyl)glycine
(3,5-dibromo-4-{[3-(cyclobutylamino)-2-fluorobenzyl]oxy}phenyl)acetic acid
3-(3,5-dichloro-4-{[2-chloro-3-(cyclobutylamino)benzyl]oxy}phenyl)propanoic acid
(3,5-dibromo-4-{[2-chloro-3-(cyclobutylamino)benzyl]oxy}phenyl)acetic acid
N-(3,5-dichloro-4-{[2-chloro-3-(cyclobutylamino)benzyl]oxy}benzoyl)glycine
(3,5-dichloro-4-{[2-chloro-3-(cyclobutylamino)benzyl]oxy}phenyl)acetic acid
3-(3,5-dichloro-4-{[3-(cyclobutylamino)-2-fluoro-5-methylbenzyl]oxy}phenyl)propanoic acid
N-(3,5-dibromo-4-{[3-(cyclobutylamino)-2-fluoro-5-methylbenzyl]oxy}benzoyl)glycine
3-(3,5-dibromo-{[3-(cyclobutylamino)-2-fluoro-5-methylbenzyl]oxy}phenyl)propanoic acid
(3,5-dichloro-4-{[3-(cyclobutylamino)-2-fluoro-5-methylbenzyl]oxy}phenyl)acetic acid
N-(3,5-dichloro-4-{[3-(cyclobutylamino)-2-fluoro-5-methylbenzyl]oxy}benzoyl)glycine
(3,5-dibromo-4-{[3-(cyclobutylamino)-2-fluoro-5-methylbenzyl]oxy}phenyl)acetic acid
3-(3,5-dichloro-4-{[5-chloro-3-(cyclobutylamino)-2-fluorobenzyl]oxy}phenyl)propanoic acid
N-(3,5-dibromo-4-{[5-chloro-3-(cyclobutylamino)-2-fluorobenzyl]oxy}benzoyl)glycine
3-(3,5-dibromo-4-{[5-chloro-3-(cyclobutylamino)-2-fluorobenzyl]oxy}phenyl)propanoic acid
(3,5-dichloro-4-{[5-chloro-3-(cyclobutylamino)-2-fluorobenzyl]oxy}phenyl)acetic acid
N-(3,5-dichloro-4-{[5-chloro-3-(cyclobutylamino)-2-fluorobenzyl]oxy}benzoyl)glycine
(3,5-dibromo-4-{[5-chloro-3-(cyclobutylamino)-2-fluorobenzyl]oxy}phenyl)acetic acid
3-(3,5-dichloro-4-{[3-(isopropylamino)-5-(trifluoromethyl)benzyl]oxy}phenyl)propanoic acid
3-(3,5-dichloro-4-{[3-chloro-5-(isopropylamino)benzyl]oxy}phenyl)propanoic acid
3-(3,5-dichloro-4-{[3-(isopropylamino)-5-(trifluoromethyl)benzyl]oxy}phenyl)-2-fluoropropanoic acid
3-(3,5-dichloro-4-{[3-chloro-5-(isopropylamino)benzyl]oxy}phenyl)-2-fluoropropanoic acid
3-(3,5-dichloro-4-{[3-(isopropylamino)-5-methylbenzyl]oxy}phenyl)-2-fluoropropanoic acid
3-(3,5-dibromo-4-{[3-(isopropylamino)-5-(trifluoromethyl)benzyl]oxy}phenyl)-2-fluoropropanoic acid
3-(3,5-dibromo-4-{[3-(isopropylamino)-5-methylbenzyl]oxy}phenyl)-2-fluoropropanoic acid
(3,5-dichloro-4-{[3-(isopropylamino)-5-(trifluoromethyl)benzyl]oxy}phenyl)acetic acid
N-(3,5-dichloro-4-{[3-(isopropylamino)-5-(trifluoromethyl)benzyl]oxy}benzoyl)glycine
(3,5-dibromo-4-{[3-(isopropylamino)-5-(trifluoromethyl)benzyl]oxy}phenyl)acetic acid
(3,5-dichloro-4-{[3-chloro-5-(isopropylamino)benzyl]oxy}phenyl)acetic acid
N-(3,5-dichloro-4-{[3-chloro-5-(isopropylamino)benzyl]oxy}benzoyl)glycine
(3,5-dibromo-4-{[3-chloro-5-(isopropylamino)benzyl]oxy}phenyl)acetic acid
(3,5-dichloro-4-{[3-(isopropylamino)-5-methylbenzyl]oxy}phenyl)acetic acid
N-(3,5-dichloro-4-{[3-(isopropylamino)-5-methylbenzyl]oxy}benzoyl)glycine
(3,5-dibromo-4-{[3-(isopropylamino)-5-methylbenzyl]oxy}phenyl)acetic acid
3-[(3,5-dichloro-4-{[3-(isopropylamino)-5-(trifluoromethyl)benzyl]oxy}phenyl)amino]-3-oxopropanoic acid
3-[(3,5-dibromo-4-{[3-(isopropylamino)-5-(trifluoromethyl)benzyl]oxy}phenyl)amino]-3-oxopropanoic acid
3-[(4-{[3-(isopropylamino)-5-(trifluoromethyl)benzyl]oxy}-3,5-dimethylphenyl)amino]-3-oxopropanoic acid
3-[(3,5-dichloro-4-{[3-chloro-5-(isopropylamino)benzyl]oxy}phenyl)amino]-3-oxopropanoic acid
3-[(4-{[3-chloro-5-(isopropylamino)benzyl]oxy}-3,5-dimethylphenyl)amino]-3-oxopropanoic acid
3-[(3,5-dichloro-4-{[3-(isopropylamino)-5-methylbenzyl]oxy}phenyl)amino]-3-oxopropanoic acid
3-[(3,5-dibromo-4-{[3-(isopropylamino)-5-methylbenzyl]oxy}phenyl)amino]-3-oxopropanoic acid
3-[(4-{[3-(isopropylamino)-5-methylbenzyl]oxy}-3,5-dimethylphenyl)amino]-3-oxopropanoic acid
(3,5-dichloro-4-{[3-(isopropylamino)benzyl]oxy}phenyl)acetic acid
N-(3,5-dichloro-4-{[3-(isopropylamino)benzyl]oxy}benzoyl)glycine
(3,5-dibromo-4-{[3-(isopropylamino)benzyl]oxy}phenyl)acetic acid
3-(3,5-dichloro-4-{[3-fluoro-5-(isopropylamino)benzyl]oxy}phenyl)propanoic acid
N-(3,5-dibromo-4-{[3-fluoro-5-(isopropylamino)benzyl]oxy}benzoyl)glycine
3-(3,5-dibromo-4-{[3-fluoro-5-(isopropylamino)benzyl]oxy}phenyl)propanoic acid
(3,5-dichloro-4-{[3-fluoro-5-(isopropylamino)benzyl]oxy}phenyl)acetic acid
N-(3,5-dichloro-4-{[3-fluoro-5-(isopropylamino)benzyl]oxy}benzoyl)glycine
(3,5-dibromo-4-{[3-fluoro-5-(isopropylamino)benzyl]oxy}phenyl)acetic acid
3-(3,5-dichloro-4-{[3-cyano-5-(isopropylamino)benzyl]oxy}phenyl)propanoic acid
N-(3,5-dibromo-4-{[3-cyano-5-(isopropylamino)benzyl]oxy}benzoyl)glycine
3-(3,5-dibromo-4-{[3-cyano-5-(isopropylamino)benzyl]oxy}phenyl)propanoic acid
(3,5-dichloro-4-{[3-cyano-5-isopropylamino)benzyl]oxy}phenyl)acetic acid
N-(3,5-dichloro-4-{[3-cyano-5-(isopropylamino)benzyl]oxy}benzoyl)glycine
(3,5-dibromo-4-{[3-cyano-5-(isopropylamino)benzyl]oxy}phenyl)acetic acid
3-(3,5-dichloro-4-{[2-fluoro-3-(isopropylamino)benzyl]oxy}phenyl)propanoic acid
N-(3,5-dibromo-4-{[2-fluoro-3-(isopropylamino)benzyl]oxy}benzoyl)glycine
3-(3,5-dibromo-4-{[2-fluoro-3-(isopropylamino)benzyl]oxy}phenyl)propanoic acid
(3,5-dichloro-4-{[2-fluoro-3-(isopropylamino)benzyl]oxy}phenyl)acetic acid
N-(3,5-dichloro-4-{[2-fluoro-3-(isopropylamino)benzyl]oxy}benzoyl)glycine
(3,5-dibromo-4-{[2-fluoro-3-(isopropylamino)benzyl]oxy}phenyl)acetic acid
3-(3,5-dichloro-4-{[2-chloro-3-(isopropylamino)benzyl]oxy}phenyl)propanoic acid
(3,5-dibromo-4-{[2-chloro-3-(isopropylamino)benzyl]oxy}phenyl)acetic acid
N-(3,5-dichloro-4-{[2-chloro-3-(isopropylamino)benzyl]oxy}benzoyl)glycine
(3,5-dichloro-4-{[2-chloro-3-(isopropylamino)benzyl]oxy}phenyl)acetic acid
3-(3,5-dichloro-4-{[2-fluoro-3-(isopropylamino)-5-methylbenzyl]oxy}phenyl)propanoic acid
N-(3,5-dibromo-4-{[2-fluoro-3-(isopropylamino)-5-methylbenzyl]oxy}benzoyl)glycine
3-(3,5-dibromo-4-{[2-fluoro-3-(isopropylamino)-5-methylbenzyl]oxy}phenyl)propanoic acid
(3,5-dichloro-4-{[2-fluoro-3-(isopropylamino)-5-methylbenzyl]oxy}phenyl)acetic acid
N-(3,5-dichloro-4-{[2-fluoro-3-(isopropylamino)-5-methylbenzyl]oxy}benzoyl)glycine
(3,5-dibromo-4-{[2-fluoro-3-(isopropylamino)-5-methylbenzyl]oxy}phenyl)acetic acid
3-(3,5-dichloro-4-{[5-chloro-2-fluoro-3-(isopropylamino)benzyl]oxy}phenyl)propanoic acid
N-(3,5-dibromo-4-{[5-chloro-2-fluoro-3-(isopropylamino)benzyl]oxy}benzoyl)glycine
3-(3,5-dibromo-4-{[5-chloro-2-fluoro-3-(isopropylamino)benzyl]oxy}phenyl)propanoic acid
(3,5-dichloro-4-{[5-chloro-2-fluoro-3-(isopropylamino)benzyl]oxy}phenyl)acetic acid
N-(3,5-dichloro-4-{[5-chloro-2-fluoro-3-(isopropylamino)benzyl]oxy}benzoyl)glycine
(3,5-dibromo-4-{[5-chloro-2-fluoro-3-(isopropylamino)benzyl]oxy}phenyl)acetic acid
3-(3,5-dichloro-4-{[3-(propylamino)-5-(trifluoromethyl)benzyl]oxy}phenyl)propanoic acid
N-(3,5-dibromo-4-{[3-(propylamino)-(trifluoromethyl)benzyl]oxy})benzoyl)glycine
3N-(3,5-dibromo-4-{[3-(propylamino)-(trifluoroethyl)benzyl]oxy})phenyl)propanoic acid
3-(3,5-dichloro-4-{[3-chloro-5-(propylamino)benzyl]oxy}phenyl)propanoic acid
N-(3,5-dibromo-4-{[3-chloro-5-(propylamino)benzyl]oxy}benzoyl)glycine
3-(3,5-dibromo-4-{[3-chloro-5-(propylamino)benzyl]oxy}phenyl)propanoic acid
3-(3,5-dichloro-4-{[3-methyl-5-(propylamino)benzyl]oxy}phenyl)propanoic acid
N-(3,5-dibromo-4-{[3-methyl-5-(propylamino)benzyl]oxy}benzoyl)glycine
3-(3,5-dibromo-4-{[3-methyl-5-(propylamino)benzyl]oxy}phenyl)propanoic acid
3-(3,5-dichloro-4-{[3-(propylamino)-5-(trifluoromethyl)benzyl]oxy}phenyl)-2-fluoropropanoic acid
3-(3,5-dichloro-4-{[3-chloro-5-(propylamino)benzyl]oxy}phenyl)-2-fluoropropanoic acid
3-(3,5-dichloro-4-{[3-methyl-5-(propylamino)benzyl]oxy}phenyl)-2-fluoropropanoic acid
3-(3,5-dibromo-4-{[3-(propylamino)-5-(trifluoromethyl)benzyl]oxy}phenyl)-2-fluoropropanoic acid
3-(3,5-dibromo-4-{[3-chloro-5-(propylamino)benzyl]oxyl)}phenyl)-2-fluoropropanoic acid
3-(3,5-dibromo-4-{[3-methyl-5-(propylamino)benzyl]oxy}phenyl)-2-fluoropropanoic acid
(3,5-dichloro-4-{[3-(propylamino)-5-(trifluoromethyl)benzyl]oxy}phenyl)acetic acid
N-(3,5-dichloro-4-{[3-(propylamino)-5-(trifluoromethyl)benzyl]oxy}benzoyl)glycine
(3,5-dibromo-4-{[3-(propylamino)-5-(trifluoromethyl)benzyl]oxy}phenyl)acetic acid
(3,5-dichloro-4-{[3-chloro-5-(propylamino)benzyl]oxy}phenyl)acetic acid
N-(3,5-dichloro-4-{[3-chloro-5-(propylamino)benzyl]oxy}benzoyl)glycine
(3,5-dibromo-4-({[3-chloro-5-(propylamino)benzyl]oxy}phenyl)acetic acid
(3,5-dichloro-4-{[3-methyl-5-(propylamino)benzyl]oxy}phenyl)acetic acid
N-(3,5-dichloro-4-{[3-methyl-5-(propylamino)benzyl]oxy}benzoyl)glycine
(3,5-dibromo-4-{[3-methyl-5-(propylamino)benzyl]oxy}phenyl)acetic acid
3-[(3,5-dichloro-4-{[3-(propylamino)-5-(trifluoromethyl)benzyl]oxy}phenyl)amino]-3-oxopropanoic acid
3-[(3,5-dibromo-4-{[3-(propylamino)-5-(trifluoromethyl)benzyl]oxy}phenyl)amino]-3-oxopropanoic acid
3-[(3,5-dimethyl-4-{[3-(propylamino)-5-(trifluoromethyl)benzyl]oxy}phenyl)amino]-3-oxopropanoic acid
3-[(3,5-dichloro-4-{[3-chloro-5-(propylamino)benzyl]oxy}phenyl)amino]-3-oxopropanoic acid
3-[(3,5-dibromo-4-{[3-chloro-5-(propylamino)benzyl]oxy}phenyl)amino]-3-oxopropanoic acid
3-[(4-{[3-chloro-5-(propylamino)benzyl]oxy}-3,5-dimethylphenyl)amino]-3-oxopropanoic acid
3-[(3,5-dichloro-4-{[3-methyl-5-(propylamino)benzyl]oxy}phenyl)amino]-3-oxopropanoic acid
3-[(3,5-dibromo-4-{[3-methyl-5-(propylamino)benzyl]oxy}phenyl)amino]-3-oxopropanoic acid
3-[(3,5-dimethyl-4-{[3-methyl-5-(propylamino)benzyl]oxy}phenyl)amino]-3-oxopropanoic acid
(3,5-dichloro-4-{[3-(propylamino)benzyl]oxy}phenyl)acetic acid
N-(3,5-dichloro-4-{[3-(propylamino)benzyl]oxy}benzoyl)glycine
(3,5-dibromo-4-{[3-(propylamino)benzyl]oxy}phenyl)acetic acid
3-(3,5-dichloro-4-{[3-fluoro-5-(propylamino)benzyl]oxy}phenyl)propanoic acid
N-(3,5-dibromo-4-{[3-fluoro-5-(propylamino)benzyl]oxy}benzoyl)glycine
3-(3,5-dibromo-4-{[3-fluoro-5-(propylamino)benzyl]oxy}phenyl)propanoic acid
(3,5-dichloro-4-{[3-fluoro-5-(propylamino)benzyl]oxy}phenyl)acetic acid
N-(3,5-dichloro-4-{[3-fluoro-5-(propylamino)benzyl]oxy}benzoyl)glycine
(3,5-dibromo-4-{[3-fluoro-5-(propylamino)benzyl]oxy}phenyl)acetic acid
3-(3,5-dichloro-4-{[3-cyano-5-(propylamino)benzyl]oxy}phenyl)propanoic acid
N-(3,5-dibromo-4-{[3-cyano-5-(propylamino)benzyl]oxy}benzoyl)glycine
3-(3,5-dibromo-4-[3-cyano-5-(propylamino)benzyl]oxy phenyl)propanoic acid
(3,5-dichloro-4-{[3-cyano-5-(propylamino)benzyl]oxy}phenyl)acetic acid
N-(3,5-dichloro-4-{[3-cyano-5-(propylamino)benzyl]oxy}benzoyl)glycine
(3,5-dibromo-4-{[3-cyano-5-(propylamino)benzyl]oxy}phenyl)acetic acid
3-(3,5-dichloro-4-{[2-fluoro-3-(propylamino)benzyl]oxy}phenyl)propanoic acid
N-(3,5-dibromo-4-{[2-fluoro-3-(propylamino)benzyl]oxy}benzoyl)glycine
3-(3,5-dibromo-4-{[2-fluoro-3-(propylamino)benzyl]oxy}phenyl)propanoic acid
(3,5-dichloro-4-{[2-fluoro-3-(propylamino)benzyl]oxy}phenyl)acetic acid
N-(3,5-dichloro-4-{[2-fluoro-3-(propylamino)benzyl]oxy}benzoyl)glycine
(3,5-dibromo-4-{[2-fluoro-3-(propylamino)benzyl]oxy}phenyl)acetic acid
3-(3,5-dichloro-4-{[2-chloro-3-(propylamino)benzyl]oxy}phenyl)propanoic acid
N-(3,5-dibromo-4-{[2-chloro-3-(propylamino)benzyl]oxy}benzoyl)glycine
3-(3,5-dibromo-4-{[2-chloro-3-(propylamino)benzyl]oxy}phenyl)propanoic acid
(3,5-dibromo-4-{[2-chloro-3-(propylamino)benzyl]oxy}phenyl)acetic acid
N-(3,5-dichloro-4-{[2-chloro-3-(propylamino)benzyl]oxy}benzoyl)glycine
(3,5-dichloro-4-{[2-chloro-3-(propylamino)benzyl]oxy}phenyl)acetic acid
3-(3,5-dichloro-4-{[2-fluoro-5-methyl-3-(propylamino)benzyl]oxy}phenyl)propanoic acid
N-(3,5-dibromo-4-{[2-fluoro-5-methyl-3-(propylamino)benzyl]oxy}benzoyl)glycine
3-(3,5-dibromo-4-{[2-fluoro-5-methyl-3-(propylamino)benzyl]oxy}phenyl)propanoic acid
(3,5-dichloro-4-{[2-fluoro-5-methyl-3-(propylamino)benzyl]oxy}phenyl)acetic acid
N-(3,5-dichloro-4-{[2-fluoro-5-methyl-3-(propylamino)benzyl]oxy}benzoyl)glycine
(3,5-dibromo-4-{[2-fluoro-5-methyl-3-(propylamino)benzyl]oxy}phenyl)acetic acid
3-(3,5-dichloro-4-{[5-chloro-2-fluoro-3-(propylamino)benzyl]oxy}phenyl)propanoic acid
N-(3,5-dibromo-4-{[5-chloro-2-fluoro-3-(propylamino)benzyl]oxy}benzoyl)glycine
3-(3,5-dibromo-4-{[5-chloro-2-fluoro-3-(propylamino)benzyl]oxy}phenyl)propanoic acid
(3,5-dichloro-4-{[5-chloro-2-fluoro-3-(propylamino)benzyl]oxy}phenyl)acetic acid
N-(3,5-dichloro-4-{[5-chloro-2-fluoro-3-(propylamino)benzyl]oxy}benzoyl)glycine
(3,5-dibromo-4-{[5-chloro-2-fluoro-3-(propylamino)benzyl]oxy}phenyl)acetic acid
3-(4-{[3-(sec-butylamino)-5-(trifluoromethyl)benzyl]oxy}-3,5-dichlorophenyl)propanoic acid
3-(4-{[3-(sec-butylamino)-5-chlorobenzyl]oxy}-3,5-dichlorophenyl)propanoic acid
3-(4-{[3-(sec-butylamino)-5-(trifluoromethyl)benzyl]oxy}-3,5-dichlorophenyl)-2-fluoropropanoic acid
3-(4-{[3-(sec-butylamino)-5-chlorobenzyl]oxy}-3,5-dichlorophenyl)-2-fluoropropanoic acid
3-(4-{[3-(sec-butylamino)-5-methylbenzyl]oxy}-3,5-dichlorophenyl)-2-fluoropropanoic acid
3-(3,5-dibromo-4-{[3-(sec-butylamino)-5-(trifluoromethyl)benzyl]oxy}phenyl)-2-fluoropropanoic acid
3-(3,5-dibromo-4-{[3-(sec-butylamino)-5-chlorobenzyl]oxy}phenyl)-2-fluoropropanoic acid
3-(3,5-dibromo-4-{[3-(sec-butylamino)-5-methylbenzyl]oxy}phenyl)-2-fluoropropanoic acid
(4-{[3-(sec-butylamino)-5-(trifluoromethyl)benzyl]oxy}-3,5-dichlorophenyl)acetic acid
N-(4-{[3-(sec-butylamino)-5-(trifluoromethyl)benzyl]oxy}-3,5-dichlorobenzoyl)glycine
(3,5-dibromo-4-{[3-(sec-butylamino)-5-(trifluoromethyl)benzyl]oxy}phenyl)acetic acid
(4-([3-(sec-butylamino)-5-chlorobenzyl]oxy)-3,5-dichlorophenyl)acetic acid
N-(4-{[3-(se-butylamino)-5-chlorobenzyl]oxy}-3,5-dichlorobenzoyl)glycine
(3,5-dibromo-4-{[3-(sec-butylamino)-5-chlorobenzyl]oxy}phenyl)acetic acid
(4-{[3-(sec-butylamino)-5-methylbenzyl]oxy}-3,5-dichlorophenyl)acetic acid
N-(4-{[3-(sec-butylamino)-5-methylbenzyl]oxy}-3,5-dichlorobenzoyl)glycine
(3,5-dibromo-4-{[3 (sec-butylamino)-5-methylbenzyl]oxy}phenyl)acetic acid
3-[(4-{[3-(sec-butylamino)-5-(trifluoromethyl)benzyl]oxy}-3,5-dichlorophenyl)amino]-3-oxopropanoic acid
3-[(3,5-dibromo-4-{[3-(sec-butylamino)-5-(trifluoromethyl)benzyl]oxy}phenyl)amino]-3-oxopropanoic acid
3-[(4-{[3-(sec-butylamino)-5-(trifluoromethyl)benzyl]oxy}-3,5-dimethylphenyl)amino]-3-oxopropanoic acid
3-[(4-{[3-(sec-butylamino)-5-chlorobenzyl]oxy}-3,5-dichlorophenyl)amino]-3-oxopropanoic acid
3-[(3,5-dibromo-4-{[3-(sec-butylamino)-5-chlorobenzyl]oxy}phenyl)amino]-3-oxopropanoic acid
3-[(4-{[3-(sec-butylamino)-5-chlorobenzyl]oxy}-3,5-dimethylphenyl)amino]-3-oxopropanoic acid
3-[(4-{[3-(sec-butylamino)-5-methyl benzyl]oxy}-3,5-dichlorophenyl)amino]-3-oxopropanoic acid
3-[(3,5-dibromo-4-{[3-(sec-butylamino)-5-methylbenzyl]oxy}phenyl)amino]-3-oxopropanoic acid
3-[(4-{[3-(sec-butylamino)-5-methylbenzyl]oxy}-3,5-dimethylphenyl)amino]-3-oxopropanoic acid
3-(4-{[3-(sec-butylamino)-5-fluorobenzyl]oxy}-3,5-dichlorophenyl)propanoic acid
N-(3,5-dibromo-4-{[3-(sec-butylamino)-5-fluorobenzyl]oxy}benzoyl)glycine
3-(3,5-dibromo-4-{[3-(sec-butylamino)-5-fluorobenzyl]oxy}phenyl)propanoic acid
3-(4-{[3-(sec-butylamino)-5-cyanobenzyl]oxy}-3,5-dichlorophenyl)propanoic acid
N-(3,5-dibromo-4-{[3-(sec-butylamino)-5-cyanobenzyl]oxy}benzoyl)glycine
3-(3,5-dibromo-4-{[3-(sec-butylamino)-5-cyanobenzyl]oxy}phenyl)propanoic acid
3-(4-{[3-(sec-butylamino)-2-fluorobenzyl]oxy}-3,5-dichlorophenyl)propanoic acid
N-(3,5-dibromo-4-{[3-(sec-butylamino)-2-fluorobenzyl]oxy)benzoyl)glycine
3-(3,5-dibromo-4-([3-(sec-butylamino)-2-fluorobenzyl]oxy}phenyl)propanoic acid
3-(4-{[3-(sec-butylamino)-2-chlorobenzyl]oxy}-3,5-dichlorophenyl)propanoic acid
N-(3,5-dibromo-4-{[3-(sec-butylamino)-2-chlorobenzyl]oxy}benzoyl)glycine
3-(4-{[3-(sec-butylamino)-2-fluoro-5-methylbenzyl]oxy}-3,5-dichlorophenyl)propanoic acid
N-(3,5-dibromo-4-{[3-(sec-butylamino)-2-fluoro-5-methylbenzyl]oxy)benzoyl)glycine
3-(3,5-dibromo-4-([3-(sec-butylamino)-2-fluoro-5-methylbenzyl]oxy}phenyl)propanoic acid
(4-{[3-(sec-butylamino)-2-fluoro-5-methylbenzyl]oxy}-3,5-dichlorophenyl)acetic acid
N-(4-{[3-(sec-butylamino)-2-fluoro-5-methylbenzyl]oxy}-3,5-dichlorobenzoyl)glycine
(3,5-dibromo-4-{[3-(sec-butylamino)-2-fluoro-5-methylbenzyl]oxy}phenyl)acetic acid
3-(4-{[3-(sec-butylamino)-5-chloro-2-fluorobenzyl]oxy}-3,5-dichlorophenyl)propanoic acid
N-(3,5-dibromo-4-([3-(sec-butylamino)-5-chloro-2-fluorobenzyl]oxy)benzoyl)glycine
3-(3,5-dibromo-4-{[3-(sec-butylamino)-5-chloro-2-fluorobenzyl]oxy}phenyl)propanoic acid
(4-{[3-(sec-butylamino)-5-chloro-2-fluorobenzyl]oxy}-3,5-dichlorophenyl)acetic acid
N-(4-{[3-(sec-butylamino)-5-chloro-2-fluorobenzyl]oxy)-3,5-dichlorobenzoyl)glycine
(3,5-dibromo-4-([3-(sec-butylamino)-5-chloro-2-fluorobenzyl]oxy}phenyl)acetic acid
N-[3,5-dibromo-4-({3-methyl-5-[(2,2,2-trifluoroethyl)amino]benzyl}oxy)benzoyl]glycine
3-[3,5-dibromo-4-({3-methyl-5-[(2,2,2-trifluoroethyl)amino]benzyl oxy}phenyl]propanoic acid
3-(3,5-dichloro-4-{[3-[(2,2,2-trifluoroethyl)amino]-5-(trifluoromethyl)benzyl]oxy}phenyl)-2-fluoropropanoic acid
{4-[(E)-2-(3-amino-phenyl)-vinyl]-3,5-dibromo-benzyloxy}-acetic acid tert-butyl ester

More preferred compounds of the invention include:
3-(3,5-dibromo-4-{[3-(methylamino)benzyl]oxy}phenyl)propanoic acid
4-[(3-aminobenzyl)oxy]-3,5-dibromobenzoic acid
3-(4-{[3-amino-5-(trifluoromethyl)benzyl]oxy}-3,5-dibromophenyl)propanoic acid
3-(3,5-dibromo-4-{[3-(methylamino)-5-(trifluoromethyl)benzyl]oxy}phenyl)propanoic acid
3-(3,5-dibromo-4-{[2-methyl-3-(methylamino)benzyl]oxy}phenyl)propanoic acid
3-(3,5-dibromo-4-{[3-(ethylamino)-5-(trifluoromethyl)benzyl]oxy}phenyl)propanoic acid
3-(4-[(3-amino-5-chlorobenzyl)oxy]-3,5-dibromophenyl propanoic acid
3-(3,5-dibromo-4-{[3-chloro-5-(ethylamino)benzyl]oxy}phenyl)propanoic acid
3-{4-[(3-amino-5-methylbenzyl)oxy]-3,5-dibromophenyl}propanoic acid
3-(3,5-dibromo-4-{[3-(ethylamino)-5-methylbenzyl]oxy}phenyl)propanoic acid
3-{4-[(3-amino-4-methylbenzyl)oxy]-3,5-dibromophenyl}propanoic acid
3-(3,5-dibromo-4-{[3-(ethylamino)-4-methylbenzyl]oxy}phenyl)propanoic acid
3-(3,5-dibromo-4-{[3-(ethylamino)-2-methylbenzyl]oxy}phenyl)propanoic acid
3-(3,5-dibromo-4-{[3-(propylamino)benzyl]oxy}phenyl)propanoic acid
3-(3,5-dibromo-4-{[2-methyl-3-(propylamino)benzyl]oxy}phenyl)propanoic acid
3-[3,5-dibromo-4-({3-[(2E)-but-2-en-1-ylamino]benzyl}oxy)phenyl]propanoic acid
3-[3,5-dibromo-4-({3-[(3,3-dimethylbutyl)amino]benzyl}oxy)phenyl]propanoic acid
3-{3,5-dibromo-4-[(3-{[2-(methylthio)ethyl]amino}benzyl)oxy]phenyl}propanoic acid
3-(3,5-dibromo-4-{[3-(isopropylamino)benzyl]oxy}phenyl)propanoic acid
3-(3,5-dibromo-4-{[3-(isopropylamino)-5-(trifluoromethyl)benzyl]oxy}phenyl)propanoic acid
3-(3,5-dibromo-4-{[3-(butylamino)benzyl]oxy}phenyl)propanoic acid
3-(3,5-dibromo-4-{[3-(sec-butylamino)benzyl]oxy}phenyl)propanoic acid
3-(3,5-dibromo-4-{[3-(sec-butylamino)-2-methylbenzyl]oxy}phenyl)propanoic acid
3-[3,5-dibromo-4-({3-[(2-ethylbutyl)amino]benzyl}oxy)phenyl]propanoic acid
3-(3,5-dibromo-4-{[3-(cyclobutylamino)benzyl]oxy}phenyl)propanoic acid
3-(3,5-dibromo-4-{[3-cyclobutylamino)-2-methylbenzyl]oxy}phenyl)propanoic acid
3-[3,5-dibromo-4-({3-[(cyclopropylmethyl)amino]benzyl}oxy)phenyl]propanoic acid
3-[3,5-dibromo-4-({3-[(cyclohexylmethyl)amino]benzyl}oxy)phenyl]propanoic acid
3-(3,5-dibromo-4-{[3-(isobutylamino)benzyl]oxy}phenyl)propanoic acid
3-{4-[(3-aminobenzyl)oxy]-3,5-dichlorophenyl}propanoic acid
3-(3,5-dichloro-4-{[3-(methylamino)benzyl]oxy}phenyl)propanoic acid
3-(3,5-dichloro-4-{[3-(ethylamino)benzyl]oxy}phenyl)propanoic acid
3-(3,5-dichloro-4-{[3-(ethylamino)-5-(trifluoromethyl)benzyl]oxy}phenyl)propanoic acid
3-(3,5-dichloro-4-{[3-(propylamino)benzyl]oxy}phenyl)propanoic acid
3-(3,5-dichloro-4-{[3-(isopropylamino)benzyl]oxy}phenyl)propanoic acid
3-(4-{[3-(sec-butylamino)benzyl]oxy}-3,5-dichlorophenyl)propanoic acid
3-[3,5-dichloro-4-({3-[(cyclopropylmethyl)amino]benzyl}oxy)phenyl]propanoic acid
N-{4-[(3-aminobenzyl)oxy]-3,5-dibromobenzoyl}glycine
N-(3,5-dibromo-4-{[3-(ethylamino)benzyl]oxy}benzoyl)glycine
N-(3,5-dibromo-4-{[3-(methylamino)benzyl]oxy}benzoyl)glycine
N-(3,5-dibromo-4-{[3-(propylamino)benzyl]oxy}benzoyl)glycine
N-(3,5-dibromo-4-{[3-(isopropylamino)benzyl]oxy}benzoyl)glycine
N-(3,5-dibromo-4-{[3-(cyclobutylamino)benzyl]oxy}benzoyl)glycine
N-(3,5-dibromo-4-{[3-(sec-butylamino)benzyl]oxy}benzoyl)glycine
N-(3,5-dichloro-4-{[3-(ethylamino)benzyl]oxy}benzoyl)glycine
N-(3,5-dibromo-4-{[3-(ethylamino)-5-(trifluoromethyl)benzyl]oxy}benzoyl)glycine
N-(3,5-dibromo-4-{[3-chloro-5-(ethylamino)benzyl]oxy}benzoyl)glycine
N-(3,5-dibromo-4-{[3-(ethylamino)-5-methylbenzyl]oxy}benzoyl)glycine
3-(3,5-dichloro-4-{[3-(ethylamino)-5-methylbenzyl]oxy}phenyl)propanoic acid
3-(3,5-dibromo-4-{[3-chloro-5-(ethylamino)benzyl]oxy}phenyl)-2-fluoropropanoic acid
15 (3,5-dichloro-4-{[3-(ethylamino)-5-methylbenzyl]oxy}phenyl)acetic acid
(3,5-dibromo-4-{[3-(ethylamino)benzyl]oxy}phenyl)acetic acid
3-(3,5-dibromo-4-{[3-cyano-5-(ethylamino)benzyl]oxy}phenyl)propanoic acid
3-(3,5-dibromo-4-{[3-(ethylamino)-2-fluorobenzyl]oxy}phenyl)propanoic acid
3-(3,5-dibromo-4-{[2-chloro-3-(ethylamino)benzyl]oxy}phenyl)propanoic acid
N-(3,5-dibromo-4-{[2-chloro-3-(ethylamino)benzyl]oxy}benzoyl)glycine
3-(3,5-dibromo-4-{[3-chloro-5-(methylamino)benzyl]oxy}phenyl)propanoic acid
3-(3,5-dibromo-4-{[3-methyl-5-(methylamino)benzyl]oxy}phenyl)propanoic acid
3-(3,5-dibromo-4-{[3-(cyclobutylamino)-5-methylbenzyl]oxy}phenyl)propanoic acid
N-(3,5-dibromo-4-{[3-(cyclobutylamino)-5-methylbenzyl]oxy}benzoyl)glycine
3-(3,5-dichloro-4-{[3-(cyclobutylamino)-5-methylbenzyl]oxy}phenyl)propanoic acid
3-(3,5-dibromo-4-{[3-chloro-5-(cyclobutylamino)benzyl]oxy}phenyl)propanoic acid
N-(3,5-dibromo-4-{[3-chloro-5-(cyclobutylamino)benzyl]oxy}benzoyl)glycine
3-(3,5-dibromo-4-{[3-(cyclobutylamino)-5-(trifluoromethyl)benzyl]oxy}phenyl)propanoic acid
N-(3,5-dibromo-4-{[3-(cyclobutylamino)-5-(trifluoromethyl)benzyl]oxy}benzoyl)glycine
3-(3,5-dibromo-4-{[3-cyano-5-(cyclobutylamino)benzyl]oxy}phenyl)propanoic acid
N-(3,5-dibromo-4-{[2-chloro-3-(cyclobutylamino)benzyl]oxy}benzoyl)glycine
3-(3,5-dibromo-4-{[2-chloro-3-(cyclobutylamino)benzyl]oxy}phenyl)propanoic acid
3-(3,5-dibromo-4-{[3-(isopropylamino)-5-methylbenzyl]oxy}phenyl)propanoic acid
N-(3,5-dibromo-{([3-(isopropylamino)-5-methylbenzyl]oxy}benzoyl)glycine
3-(3,5-dichloro-4-{[3-(isopropylamino)-5-methylbenzyl]oxy}phenyl)propanoic acid
3-(3,5-dibromo-4-{[3-chloro-5-(isopropylamino)benzyl]oxy}phenyl)propanoic acid
N-(3,5-dibromo-4-{[3-chloro-5-(isopropylamino)benzyl]oxy}benzoyl)glycine
N-(3,5-dibromo-4-([3-(isopropylamino)-5-(trifluoromethyl)benzyl]oxy benzoyl)glycine
3-(3,5-dibromo-4-{[3-chloro-5-(isopropylamino)benzyl]oxy}phenyl)-2-fluoropropanoic acid
N-(3,5-dibromo-4-{[2-chloro-3-(isopropylamino)benzyl]oxy}benzoyl)glycine
3-(3,5-dibromo-4-{[2-chloro-3-(isopropylamino)benzyl]oxy}phenyl)propanoic acid
3-(3,5-dibromo-4-{[3-(sec-butylamino)-5-methylbenzyl]oxy}phenyl)propanoic acid
N-(3,5-dibromo-4-{[3-(sec-butylamino)-5-methylbenzyl]oxy}benzoyl)glycine
3-(4-{[3-(sec-butylamino)-5-methylbenzyl]oxy}-3,5-dichlorophenyl)propanoic acid
3-(3,5-dibromo-4-{[3-(sec-butylamino)-5-chlorobenzyl]oxy}phenyl)propanoic acid
N-(3,5-dibromo-4-{[3-(sec-butylamino)-5-chlorobenzyl]oxy}benzoyl)glycine
3-(3,5-dibromo-4-{[3-(sec-butylamino)-5-(trifluoromethyl)benzyl]oxy}phenyl)propanoic acid
N-(3,5-dibromo-4-{[3-(sec-butylamino)-5-(trifluoromethyl)benzyl]oxy}benzoyl)glycine
3-(3,5-dibromo-4-{[3-(sec-butylamino)-2-chlorobenzyl]oxy}phenyl)propanoic acid
(3,5-dibromo-4-{[3-chloro-5-(ethylamino)benzyl]oxy}phenyl)acetic acid
(3,5-dichloro-4-{[3-(ethylamino)-5-methyl-benzyl]oxy}phenyl)acetic acid
[4-(3-amino-5-methylbenzyloxy)-3,5-dichlorophenyl]acetic acid
N-(3,5-dichloro-4-{[3-(ethylamino)-5-methylbenzyl]oxy}benzoyl)glycine
N-(4-{[3-amino-5-methylbenzyl)oxy]-3,5-dichlorobenzoyl}glycine
3-(3,5-dibromo-4-{[3-(ethylamino)-5-(fluoromethyl)benzyl]oxy}phenyl)propanoic acid
3-(3,5-dibromo-4-{[3-ethoxymethyl-5-(ethylamino)benzyl]oxy}phenyl)propanoic acid
3-(4-[3-amino-2-chlorobenzyloxy]-3,5-bromophenyl) propanoic acid
3-(4-[3-amino-2-fluorobenzyloxy]-3,5-bromophenyl) propanoic acid
3-(3,5-dibromo-4-{[2-ethoxy-3-(ethylamino)benzyl]oxy}phenyl)propanoic acid
3-(3,5-dibromo-4-{[2-methoxy-3-(ethylamino)benzyl]oxy}phenyl)propanoic acid
3-(4-{[3-(2-carboxy-ethylamino)benzyl]oxy}-3,5-dichlorophenyl)propanoic acid
3-[3,5-dichloro-4-({3-[(cyclopropyl)amino]benzyl}oxy)phenyl]propanoic acid
3-(3,5-dibromo-4-{[2-fluoro-5-(isopropylamino)benzyl]oxy}phenyl)propanoic acid
3-(3,5-dibromo-4-{[2-fluoro-5-(cyclobutylamino)benzyl]oxy}phenyl)propanoic acid
3-(3,5-dibromo-4-{[2-fluoro-5-(sec-butylamino)benzyl]oxy}phenyl)propanoic acid
3-(3,5-dibromo-4-{[2-fluoro-5-(ethylamino)benzyl]oxy}phenyl)propanoic acid
3-(3,5-dibromo-4-{[2,5-dichloro-3-(ethylamino)benzyl]oxy}phenyl)propanoic acid
3-(3,5-dibromo-4-{[2,5-dichloro-3-(isopropylamino)benzyl]oxy}phenyl)propanoic acid
3-(3,5-dibromo-4-{[2,5-dichloro-3-(cyclobutylamino)benzyl]oxy}phenyl)propanoic acid
3-(3,5-dibromo-4-{[3-chloro-5-(1,2-dimethyl-propylamino)benzyl]oxy}phenyl)propanoic acid
3-(3,5-dibromo-4-{[2-chloro-3-(1,2-dimethyl-propylamino)benzyl]oxy}phenyl)propanoic acid
N-(3,5-dibromo-4-{[3-chloro-5-(1,2-dimethyl-propylamino)benzyl]oxy}benzoyl)glycine
3-(3,5-dibromo-4-{[2,5-dichloro-3-(isopropylamino)benzyl]oxy}phenyl)-2-fluoropropanoic acid
3-(3,5-dibromo-4-{[2,5-dichloro-3-(ethylamino)benzyl]oxy}phenyl)-2-fluoropropanoic acid
3-(3,5-dibromo-4-{[2-chloro-3-(ethylamino)benzyl]oxy}phenyl)-2-fluoropropanoic acid
3-(3,5-dibromo-4-{[3-(ethylamino)-5-methylbenzyl]oxy}phenyl)butanoic acid
3-(3,5-dibromo-4-{[3-(isopropylamino)-5-methylbenzyl]oxy}phenyl)butanoic acid
3-(3,5-dibromo-4-{[3-(cyclobutylamino)-5-methylbenzyl]oxy}phenyl)butanoic acid
3-(3,5-dibromo-4-{[2,5-dichloro-3-(ethylamino)benzyl]oxy}phenyl)butanoic acid
(E)-3-(3,5-dibromo-4-{[2,5-dichloro-3-(ethylamino)benzyl]oxy}phenyl)acrylic acid
(E)-3-(3,5-dibromo-4-{[2,5-dichloro-3-(isopropylamino)benzyl]oxy}phenyl)acrylic acid
(E)-3-(3,5-dibromo-4-{[2,5-dichloro-3-(cyclobutylamino)benzyl]oxy}phenyl)acrylic acid
N-(3,5-dibromo-4-{[2,5-dichloro-3-(ethylamino)benzyl]oxy}benzoyl)glycine
N-(3,5-dibromo-4-{[2,5-dichloro-3-(isopropylamino)benzyl]oxy}benzoyl)glycine
N-(3,5-dibromo-4-{[2,5-dichloro-3-(cyclobutylamino)benzyl]oxy}benzoyl)glycine
(S)-2-{2-(3,5-dibromo-4-{[3-chloro-5-(ethylamino)benzyl]oxy}phenyl)acetylamino}-3-phenyl-propanoic acid
(S)-2-{2-(3,5-dichloro-4-{[3-(ethylamino)-5-methylbenzyl]oxy}phenyl)acetylamino}-2-phenyl-acetic acid
3-[(3,5-dibromo-4-{[3-chloro-5-(isopropylamino)benzyl]oxy}phenyl)amino]-3-oxopropanoic acid
3-(3,5-dichloro-4-{[3-chloro-5-(ethylamino)benzyl]oxy}phenyl)propanoic acid
(3,5-dibromo-4-{[3-(ethylamino)-5-fluorobenzyl]oxy}phenyl)acetic acid
3-(3,5-dichloro-4-{[3-cyano-5-(ethylamino)benzyl]oxy}phenyl)propanoic acid
3-(3,5-dichloro-4-{[3-chloro-5-(ethylamino)benzyl]oxy}phenyl)-2-fluoropropanoic acid
3-(3,5-dichloro-4-{[3-(ethylamino)-5-methylbenzyl]oxy}phenyl)-2-fluoropropanoic acid
3-(3,5-dibromo-4-{[3-(ethylamino)-5-methylbenzyl]oxy}phenyl)-2-fluoropropanoic acid
3-[(3,5-dibromo-4-{[3-(ethylamino)-5-(trifluoromethyl)benzyl]oxy}phenyl)amino]-3-oxopropanoic acid
3-(3,5-dibromo-4-{[3-(ethylamino)-5-fluorobenzyl]oxy}phenyl)propanoic acid
3-(3,5-dichloro-4-{[3-(ethylamino)-2-fluorobenzyl]oxy}phenyl)propanoic acid
N-(3,5-dibromo-4-{[3-(ethylamino)-2-fluorobenzyl]oxy}benzoyl)glycine
3-(3,5-dichloro-4-{[2-chloro-3-(ethylamino)benzyl]oxy)phenyl)propanoic acid
3-(3,5-dichloro-4-([3-(ethylamino)-2-fluoro-5-methylbenzyl]oxy}phenyl)propanoic acid
N-(3,5-dibromo-4-{[3-(ethylamino)-2-fluoro-5-methylbenzyl]oxy}benzoyl)glycine
3-(3,5-dibromo-4-{[3-(ethylamino)-2-fluoro-5-methylbenzyl]oxy}phenyl)propanoic acid
3-(3,5-dichloro-4-{[5-chloro-3-(ethylamino)-2-fluorobenzyl]oxy}phenyl)propanoic acid
N-(3,5-dibromo-4-{[5-chloro-3-(ethylamino)-2-fluorobenzyl]oxy}benzoyl)glycine
3-(3,5-dibromo-4-{[5-chloro-3-(ethylamino)-2-fluorobenzyl]oxy}phenyl)propanoic acid
3-(3,5-dichloro-4-[3-(methylamino)-5-(trifluoromethyl)benzyl]oxy)phenyl)propanoic acid
N-(3,5-dibromo-4-{[3-(methylamino)-5-(trifluoromethyl)benzyl]oxy}benzoyl)glycine
3-(3,5-dichloro-4-{[3-chloro-5-(methylamino)benzyl]oxy}phenyl)propanoic acid
N-(3,5-dibromo-4-{[3-chloro-5-(methylamino)benzyl]oxy}benzoyl)glycine
3-(3,5-dichloro-4-{[3-methyl-5-(methylamino)benzyl]oxy}phenyl)propanoic acid
N-(3,5-dibromo-4-{[3-methyl-5-(methylamino)benzyl]oxy}benzoyl)glycine
3-(3,5-dichloro-4-{[3-(methylamino)-5-(trifluoromethyl)benzyl]oxy}phenyl)-2-fluoropropanoic acid
3-(3,5-dichloro-4-{[3-chloro-5-(methylamino)benzyl]oxy}phenyl)-2-fluoropropanoic acid
3-(3,5-dichloro-4-{[3-methyl-5-(methylamino)benzyl]oxy}phenyl)-2-fluoropropanoic acid
3-(3,5-dibromo-4-{[3-(methylamino)-5-(trifluoromethyl)benzyl]oxy}phenyl)-2-fluoropropanoic acid
3-(3,5-dibromo-4-{[3-chloro-5-(methylamino)benzyl]oxy}phenyl)-2-fluoropropanoic acid
3-(3,5-dibromo-4-{[3-methyl-5-(methylamino)benzyl]oxy}phenyl)-2-fluoropropanoic acid
(3-[(3,5-dichloro-4-{[3-(methylamino)-5-(trifluoromethyl)benzyl]oxy}phenyl)amino]-3-oxopropanoic acid
3-[(3,5-dibromo-4-{[3-(methylamino)-5-(trifluoromethyl)benzyl]oxy}phenyl)amino]-3-oxopropanoic acid
3-[(3,5-dimethyl-4-{[3-(methylamino)-5-(trifluoromethyl)benzyl]oxy}phenyl)amino]-3-oxopropanoic acid
3-[(3,5-dichloro-4-{[3-chloro-5-(methylamino)benzyl]oxy}phenyl)amino]-3-oxopropanoic acid
3-[(3,5-dibromo-4-{[3-chloro-5-(methylamino)benzyl]oxy}phenyl)amino]-3-oxopropanoic acid
3-[(4-{[3-chloro-5-(methylamino)benzyl]oxy}-3,5-dimethylphenyl)amino]-3-oxopropanoic acid
3-[(3,5-dichloro-4-{[3-methyl-5-(methylamino)benzyl]oxy}phenyl)amino]-3-oxopropanoic acid
3-[(3,5-dibromo-4-{[3-methyl-5-(methylamino)benzyl]oxy}phenyl)amino]-3-oxopropanoic acid
3-[(3,5-dimethyl-4-{[3-methyl-5-(methylamino)benzyl]oxy}phenyl)amino]-3-oxopropanoic acid
3-(3,5-dibromo-4-{[2-fluoro-3-(methylamino)benzyl]oxy}phenyl)propanoic acid
3-(3,5-dichloro-4-{[2-chloro-3-(methylamino)benzyl]oxy}phenyl)propanoic acid
N-(3,5-dibromo-4-{[2-chloro-3-(methylamino)benzyl]oxy}benzoyl)glycine
3-(3,5-dibromo-4-{[2-chloro-3-(methylamino)benzyl]oxy}phenyl)propanoic acid
3-(3,5-dichloro-4-{[2-fluoro-5-methyl-3-(methylamino)benzyl]oxy}phenyl)propanoic acid
N-(3,5-dibromo-4-{[2-fluoro-5-methyl-3-(methylamino)benzyl]oxy}benzoyl)glycine
3-(3,5-dibromo-4-{[2-fluoro-5-methyl-3-(methylamino)benzyl]oxy}phenyl)propanoic acid
3-(3,5-dichloro-4-{[5-chloro-2-fluoro-3-(methylamino)benzyl]oxy}phenyl)propanoic acid
N-(3,5-dibromo-4-([5-chloro-2-fluoro-3-(methylamino)benzyl]oxy)benzoyl)glycine
3-(3,5-dibromo-4-{[5-chloro-2-fluoro-3-(methylamino)benzyl]oxy}phenyl)propanoic acid
3-[3,5-dichloro-4-({3-chloro-5-[(cyclopropylmethyl)amino]benzyl}oxy)phenyl]propanoic acid
N-[3,5-dibromo-4-({3-chloro-5-[(cyclopropylmethyl)amino]benzyl}oxy)benzoyl]glycine
3-[3,5-dibromo-4-({3-chloro-5-[(cyclopropylmethyl)amino]benzyl}oxy)phenyl]propanoic acid
3-[3,5-dichloro-4-({3-[(cyclopropylmethyl)amino]-5-methylbenzyl}oxy)phenyl]propanoic acid
N-[3,5-dibromo-4-({3-[(cyclopropylmethyl)amino]-5-methylbenzyl}oxy)benzoyl]glycine
3-[3,5-dibromo-4-({3-[(cyclopropylmethyl)amino]-5-methylbenzyloxy}phenyl]propanoic acid
3-[3,5-dibromo-4-({3-[(cyclopropylmethyl)amino]-5-methylbenzyl}oxy)phenyl]-2-fluoropropanoic acid
3-([3,5-d]chloro-4-({3-chloro-5-[(cyclopropylmethyl)amino-3 benzyl}oxy)phenyl]amino)-3-oxopropanoic acid
3-{[3,5-dibromo-4-({3-chloro-5-[(cyclopropylmethyl)amino]benzyl}oxy)phenyl]amino}-3-oxopropanoic acid
3-{[3,5-dibromo-4-({3-[(cyclopropylmethyl)amino]-5-methylbenzyl}oxy)phenyl]amino}-3-oxopropanoic acid
3-[3,5-dichloro-4-({3-[(cyclopropylmethyl)amino]-5-fluorobenzyl}oxy)phenyl]propanoic acid
3-[3,5-dibromo-4-({3-[(cyclopropylmethyl)amino]-2-fluorobenzyl}oxy)phenyl]propanoic acid
N-[3,5-dibromo-4-({2-chloro-3-[(cyclopropylmethyl)amino]benzyl}oxy)benzoyl]glycine
3-[3,5-dibromo-4-({2-chloro-3-[(cyclopropylmethyl)amino]benzyl}oxy)phenyl]propanoic acid
3-[3,5-dichloro-4-({3-[(cyclopropylmethyl)amino]-2-fluoro-5-methylbenzyl}oxy)phenyl]propanoic acid
N-[3,5-dibromo-4-({3-[(cyclopropylmethyl)amino]-2-fluoro-5-methylbenzyl}oxy)benzoyl]glycine
3-[3,5-dibromo-4-({3-[(cyclopropylmethyl)amino]-2-fluoro-5-methylbenzyl}oxy)phenyl]propanoic acid
3-[3,5-dichloro-4-({5-chloro-3-[(cyclopropylmethyl)amino]-2-fluorobenzyl}oxy)phenyl]propanoic acid
N-[3,5-dibromo-4-({S-chloro-3-[(cyclopropylmethyl)amino]-2-fluorobenzyl}oxy)benzoyl]glycine
3-[3,5-dibromo-4-({5-chloro-3-[(cyclopropylmethyl)amino]-2-fluorobenzyl}oxy)phenyl]propanoic acid
3-(3,5-dichloro-4-{[3-chloro-5-(cyclobutylamino)benzyl]oxy}phenyl)propanoic acid
3-(3,5-dichloro-4-{[3-(cyclobutylamino)-5-(trifluoromethyl)benzyl]oxy}phenyl)-2-fluoropropanoic acid
3-(3,5-dichloro-4-{[3-chloro-5-(cyclobutylamino)benzyl]oxy}phenyl)-2-fluoropropanoic acid
3-(3,5-dichloro-4-{[3-(cyclobutylamino)-5-methylbenzyl]oxy}phenyl)-2-fluoropropanoic acid
3-(3,5-dibromo-4-{[3-(cyclobutylamino)-5-(trifluoromethyl)benzyl]oxy}phenyl)-2-fluoropropanoic acid
3-(3,5-dibromo-4-{[3-chloro-5-(cyclobutylamino)benzyl]oxy}phenyl)-2-fluoropropanoic acid
3-(3,5-dibromo-4-{[3-(cyclobutylamino)-5-methylbenzyl]oxy}phenyl)-2-fluoropropanoic acid
3-[(3,5-dichloro-4-{[3-(cyclobutylamino)-5-methylbenzyl]oxy}phenyl)amino]-3-oxopropanoic acid
3-[(3,5-dibromo-4-{[3-(cyclobutylamino)-5-methylbenzyl]oxy}phenyl)amino]-3-oxopropanoic acid
3-(3,5-dichloro-4-{[3-(cyclobutylamino)-2-fluorobenzyl]oxy}phenyl)propanoic acid
N-(3,5-dibromo-4-{[3-(cyclobutylamino)-2-fluorobenzyl]oxy}benzoyl)glycine
3-(3,5-dibromo-4-{[3-(cyclobutylamino)-2-fluorobenzyl]oxy}phenyl)propanoic acid
3-(3,5-dichloro-4-{[2-chloro-3-(cyclobutylamino)benzyl]oxy}phenyl)propanoic acid
3-(3,5-dichloro-4-{[3-(cyclobutylamino)-2-fluoro-5-methylbenzyl]oxy}phenyl)propanoic acid
N-(3,5-dibromo-4-{[3-(cyclobutylamino)-2-fluoro-5-methylbenzyl]oxy}benzoyl)glycine
3-(3,5-dibromo-4-{[3-(cyclobutylamino)-2-fluoro-5-methylbenzyl]oxy}phenyl)propanoic acid
3-(3,5-dichloro-4-{[5-chloro-3-(cyclobutylamino)-2-fluorobenzyl]oxy}phenyl)propanoic acid
N-(3,5-dibromo-4-{[5-chloro-3-(cyclobutylamino)-2-fluorobenzyl]oxy}benzoyl)glycine
3-(3,5-dibromo-4-{[5-chloro-3-(cyclobutylamino)-2-fluorobenzyl]oxy}phenyl)propanoic acid
3-(3,5-dichloro-4-{[3-(isopropylamino)-5-(trifluoromethyl)benzyl]oxy}phenyl)propanoic acid
3-(3,5-dichloro-4-{[3-chloro-5-(isopropylamino)benzyl]oxy}phenyl)propanoic acid
3-(3,5-dibromo-4-{[3-(isopropylamino)-5-methylbenzyl]oxy}phenyl)-2-fluoropropanoic acid
3-[(3,5-dichloro-4-{[3-chloro-5-(isopropylamino)benzyl]oxy}phenyl)amino]-3-oxopropanoic acid
3-[(3,5-dichloro-4-{[3-(isopropylamino)-5-methylbenzyl]oxy}phenyl)amino]-3-oxopropanoic acid
3-[(3,5-dibromo-4-{[3-(isopropylamino)-5-methylbenzyl]oxy}phenyl)amino]-3-oxopropanoic acid
3-(3,5-dibromo-4-{[3-fluoro-5-(isopropylamino)benzyl]oxy}phenyl)propanoic acid
3-(3,5-dichloro-4-{[2-fluoro-3-(isopropylamino)benzyl]oxy}phenyl)propanoic acid
N-(3,5-dibromo-4-{[2-fluoro-3-(isopropylamino)benzyl]oxy}benzoyl)glycine
3-(3,5-dibromo-4-{[2-fluoro-3-(isopropylamino)benzyl]oxy}phenyl)propanoic acid
3-(3,5-dichloro-4-{[2-chloro-3-(isopropylamino)benzyl]oxy}phenyl)propanoic acid
3-(3,5-dichloro-4-{[2-fluoro-3-(isopropylamino)-5-methylbenzyl]oxy}phenyl)propanoic acid
N-(3,5-dibromo-4-{[2-fluoro-3-(isopropylamino)-5-methylbenzyl]oxy}benzoyl)glycine
3-(3,5-dibromo-4-{[2-fluoro-3-(isopropylamino)-5-methylbenzyl]oxy}phenyl)propanoic acid
3-(3,5-dichloro-4-{[5-chloro-2-fluoro-3-(isopropylamino)benzyl]oxy}phenyl)propanoic acid
N-(3,5-dibromo-4-{([5-chloro-2-fluoro-3-(isopropylamino)benzyl]oxy}benzoyl)glycine
3-(3,5-dibromo-4-{[5-chloro-2-fluoro-3-(isopropylamino)benzyl]oxy}phenyl)propanoic acid
3-(3,5-dichloro-4-{[3-chloro-5-(propylamino)benzyl]oxy}phenyl)propanoic acid
N-(3,5-dibromo-4-{[3-chloro-5-(propylamino)benzyl]oxy}benzoyl)glycine
3-(3,5-dibromo-4-{[3-chloro-5-(propylamino)benzyl]oxy}phenyl)propanoic acid
3-(3,5-dichloro-4-{[3-methyl-5-(propylamino)benzyl]oxy}phenyl)propanoic acid
N-(3,5-dibromo-4-{[3-methyl-5-(propylamino)benzyl]oxy}benzoyl)glycine
3-(3,5-dibromo-4-{[3-methyl-5-(propylamino)benzyl]oxy}phenyl)propanoic acid
3-(3,5-dichloro-4-{[2-fluoro-3-(propylamino)benzyl]oxy}phenyl)propanoic acid
3-(3,5-dibromo-4-{[2-fluoro-3-(propylamino)benzyl]oxy}phenyl)propanoic acid
3-(3,5-dichloro-4-{[2-chloro-3-(propylamino)benzyl]oxy}phenyl)propanoic acid
3-(3,5-dibromo-4-{[2-fluoro-5-methyl-3-(propylamino)benzyl]oxy}phenyl)propanoic acid
3-(3,5-dibromo-4-([5-chloro-2-fluoro-3-(propylamino)benzyl]oxy phenyl)propanoic acid
3-(4-t[3-(sec-butylamino)-5-(trifluoromethyl)benzyl]oxy)-3,5-dichlorophenyl)-2-fluoropropanoic acid
3-(4-{[3-(sec-butylamino)-5-chlorobenzyl]oxy}-3,5-dichlorophenyl)-2-fluoropropanoic acid
3-(4-{[3-(sec-butylamino)-5-methylbenzyl]oxy}-3,5-dichlorophenyl)-2-fluoropropanoic acid
3-(3,5-dibromo-4-{[3-(sec-butylamino)-5-(trifluoromethyl)benzyl]oxy}phenyl)-2-fluoropropanoic acid
3-(3,5-dibromo-4-{[3-(sec-butylamino)-5-chlorobenzyl]oxy}phenyl)-2-fluoropropanoic acid
3-(3,5-dibromo-4-{[3-(sec-butylamino)-5-methylbenzyl]oxy}phenyl)-2-fluoropropanoic acid
3-[(4-{[3-(sec-butylamino)-5-chlorobenzyl]oxy}-3,5-dichlorophenyl)amino]-3-oxopropanoic acid
3-[(3,5-dibromo-4-{[3-(sec-butylamino)-5-chlorobenzyl]oxy}phenyl)amino]-3-oxopropanoic acid
3-[(4-{[3-(sec-butylamino)-5-chlorobenzyl]oxy}-3,5-dimethylphenyl)amino]-3-oxopropanoic acid
3-[(4-{[3-(sec-butylamino)-5-methylbenzyl]oxy}-3,5-dichlorophenyl)amino]-3-oxopropanoic acid
3-[(3,5-dibromo-4-{[3-(sec-butylamino)-5-methylbenzyl]oxy}phenyl)amino]-3-oxopropanoic acid
3-[(4-{[3-(sec-butylamino)-5-methylbenzyl]oxy}-3,5-dimethylphenyl)amino]-3-oxopropanoic acid
3-(4-{[3-(sec-butylamino)-5-fluorobenzyl]oxy}-3,5-dichlorophenyl)propanoic acid
N-(3,5-dibromo-4-{[3-(sec-butylamino)-5-fluorobenzyl]oxy}benzoyl)glycine
3-(3,5-dibromo-4-{[3-(sec-butylamino)-5-fluorobenzyl]oxy}phenyl)propanoic acid
N-(4-{[3-(sec-butylamino)-5-chloro-2-fluorobenzyl]oxy}-3,5-dichlorobenzoyl)glycine
(3,5-dibromo-4-{[3-(sec-butylamino)-5-chloro-2-fluorobenzyl]oxy}phenyl)acetic acid
3-(4-{[3-(sec-butylamino)-5-cyanobenzyl]oxy}-3,5-dichlorophenyl)propanoic acid
N-(3,5-dibromo-4-{[3-(sec-butylamino)-5-cyanobenzyl]oxy}benzoyl)glycine
3-(3,5-dibromo-4-{[3-(sec-butylamino)-5-cyanobenzyl]oxy}phenyl)propanoic acid
3-(4-{[3-(sec-butylamino)-2-fluorobenzyl]oxy}-3,5-dichlorophenyl)propanoic acid
N-(3,5-dibromo-4-{[3-(sec-butylamino)-2-chlorobenzyl]oxy}benzoyl)glycine
3-(4-{[3-(sec-butylamino)-2-fluoro-5-methylbenzyl]oxy}-3,5-dichlorophenyl)propanoic acid
N-(3,5-dibromo-4-{[3-(sec-butylamino)-2-fluoro-5-methylbenzyl]oxy}benzoyl)glycine
3-(3,5-dibromo-4-{[3-(sec-butylamino)-2-fluoro-5-methylbenzyl]oxy}phenyl)propanoic acid
3-(3,5-dibromo-4-{[3-(sec-butylamino)-5-chloro-2-fluorobenzyl]oxy}phenyl)propanoic acid
N-[3,5-dibromo-4-({3-methyl-5-[(2,2,2-trifluoroethyl)amino]benzyl}oxy)benzoyl]glycine
3-[3,5-dibromo-4-({3-methyl-5-[(2,2,2-trifluoroethyl)amino]benzyl oxy)phenyl]propanoic acid
3-(3,5-dibromo-4-([3-(cyclopropylamino)benzyl]oxy}phenyl)propanoic acid
{4-[(E)-2-(3-amino-phenyl)-vinyl]-3,5-dibromo-benzyloxy}-acetic acid tert-butyl ester

Most preferred compounds according to the invention include:
3-(3,5-dibromo-4-{[3-(methylamino)benzyl]oxy}phenyl)propanoic acid
4-[(3-aminobenzyl)oxy]-3,5-dibromobenzoic acid
3-(4-{[3-amino-5-(trifluoromethyl)benzyl]oxy}-3,5-dibromophenyl)propanoic acid
3-(3,5-dibromo-4-{[3-(methylamino)-5-(trifluoromethyl)benzyl]oxy}phenyl)propanoic acid
3-(3,5-dibromo-4-{[2-methyl-3-(methylamino)benzyl]oxy}phenyl)propanoic acid
3-(3,5-dibromo-4-{[3-(ethylamino)-5-(trifluoromethyl)benzyl]oxy}phenyl)propanoic acid
3-{4-[(3-amino-5-chlorobenzyl)oxy]-3,5-dibromophenyl}propanoic acid
3-(3,5-dibromo-4-{[3-chloro-5-(ethylamino)benzyl]oxy}phenyl)propanoic acid
3-{4-[(3-amino-5-methylbenzyl)oxy]-3,5-dibromophenyl}propanoic acid
3-(3,5-dibromo-4-{[3-(ethylamino)-5-methylbenzyl]oxy}phenyl)propanoic acid
3-{4-[(3-amino-4-methylbenzyl)oxy]-3,5-dibromophenyl}propanoic acid
3-(3,5-dibromo-4-{[3-(ethylamino)-4-methylbenzyl]oxy}phenyl)propanoic acid
3-(3,5-dibromo-4-{[3-(ethylamino)-2-methylbenzyl]oxy}phenyl)propanoic acid
3-(3,5-dibromo-4-{[3-(propylamino)benzyl]oxy}phenyl)propanoic acid
3-(3,5-dibromo-4-{[2-methyl-3-(propylamino)benzyl]oxy}phenyl)propanoic acid
3-[3,5-dibromo-4-({3-[(2E)-but-2-en-1-ylamino]benzyl}oxy)phenyl]propanoic acid
3-[3,5-dibromo-4-({3-[(3,3-dimethylbutyl)amino]benzyl}oxy)phenyl]propanoic acid
3-{3,5-dibromo-4-[(3-{[2-(methylamino)ethyl]amino}benzyl)oxy]phenyl}propanoic acid
3-(3,5-dibromo-4-{[3-(isopropylamino)benzyl]oxy}phenyl)propanoic acid
3-(3,5-dibromo-4-{[3-(isopropylamino)-5-(trifluoromethyl)benzyl]oxy}phenyl)propanoic acid
3-(3,5-dibromo-4-{[3-(butylamino)benzyl]oxy}phenyl)propanoic acid
3-(3,5-dibromo-4-{[3-(sec-butylamino)benzyl]oxy}phenyl)propanoic acid
3-(3,5-dibromo-4-{[3-(sec-butylamino)-2-methylbenzyl]oxy}phenyl)propanoic acid
3-[3,5-dibromo-4-({3-[(2-ethylbutyl)amino]benzyl}oxy)phenyl]propanoic acid
3-(3,5-dibromo-4-{[3-(cyclobutylamino)benzyl]oxy}phenyl)propanoic acid
3-(3,5-dibromo-4-{[3-(cyclobutylamino)-2-methylbenzyl]oxy}phenyl)propanoic acid
3-[3,5-dibromo-4-({3-[(cyclopropylmethyl)amino]benzyl}oxy)phenyl]propanoic acid
3-[3,5-dibromo-4-({3-[(cyclohexylmethyl)amino]benzyl}oxy)phenyl]propanoic acid
3-(3,5-dibromo-4-{[3-(isobutylamino)benzyl]oxy}phenyl)propanoic acid
3-{4-[(3-aminobenzyl)oxy]-3,5-dichlorophenyl}propanoic acid
3-(3,5-dichloro-4-{[3-(methylamino)benzyl]oxy}phenyl)propanoic acid
3-(3,5-dichloro-4-{[3-(ethylamino)benzyl]oxy}phenyl)propanoic acid
3-(3,5-dichloro-4-{[3-(ethylamino)-5-(trifluoromethyl)benzyl]oxy}phenyl)propanoic acid
3-(3,5-dichloro-4-{[3-(propylamino)benzyl]oxy}phenyl)propanoic acid
3-(3,5-dichloro-4-{[3-(isopropylamino)benzyl]oxy}phenyl)propanoic acid
3-(4-{[3-(sec-butylamino)benzyl]oxy}-3,5-dichlorophenyl)propanoic acid
3-[3,5-dichloro-4-({3-[(cyclopropylmethyl)amino]benzyl}oxy)phenyl]propanoic acid
N-{4-[(3-aminobenzyl)oxy]-3,5-dibromobenzoyl}glycine
N-(3,5-dibromo-4-{[3-(ethylamino)benzyl]oxy}benzoyl)glycine
N-(3,5-dibromo-4-{[3-(methylamino)benzyl]oxy}benzoyl)glycine
N-(3,5-dibromo-4-{[3-(propylamino)benzyl]oxy}benzoyl)glycine
N-(3,5-dibromo-4-{[3-(isopropylamino)benzyl]oxy}benzoyl)glycine
N-(3,5-dibromo-4-{[3-(cyclobutylamino)benzyl]oxy}benzoyl)glycine
N-(3,5-dibromo-4-([3-(sec-butylamino)benzyl]oxy)benzoyl)glycine
N-(3,5-dichloro-4-{[3-(ethylamino)benzyl]oxy)benzoyl)glycine
N-(3,5-dibromo-4-[3-(ethylamino)-5-(trifluoromethyl)benzyl]oxy}benzoyl)glycine
N-(3,5-dibromo-4-{[3-chloro-5-(ethylamino)benzyl]oxy)benzoyl)glycine
N-(3,5-dibromo-4-([3-(ethylamino)-5-methylbenzyl]oxy}benzoyl)glycine
3-(3,5-dichloro-4-{[3-(ethylamino)-5-methylbenzyl]oxy}phenyl)propanoic acid
3-(3,5-dibromo-4-{[3-chloro-5-(ethylamino)benzyl]oxy}phenyl)-2-fluoropropanoic acid
(3,5-dichloro-4-{[3-(ethylamino)-5-methylbenzyl]oxy}phenyl)acetic acid
(3,5-dibromo-4-{[3-(ethylamino)benzyl]oxy}phenyl)acetic acid
3-(3,5-dibromo-4-{[3-cyano-5-(ethylamino)benzyl]oxy}phenyl)propanoic acid
3-(3,5-dibromo-4-{[3-(ethylamino)-2-fluorobenzyl]oxy}phenyl)propanoic acid
3-(3,5-dibromo-4-{[2-chloro-3-(ethylamino)benzyl]oxy}phenyl)propanoic acid
N-(3,5-dibromo-4-{[2-chloro-3-(ethylamino)benzyl]oxy}benzoyl)glycine
3-(3,5-dibromo-4-{[3-chloro-5-(methylamino)benzyl]oxy}phenyl)propanoic acid
3-(3,5-dibromo-4-{[3-methyl-5-(methylamino)benzyl]oxy}phenyl)propanoic acid
3-(3,5-dibromo-4-{[3-(cyclobutylamino)-5-methylbenzyl]oxy}phenyl)propanoic acid
N-(3,5-dibromo-4-{[3-(cyclobutylamino)-5-methylbenzyl]oxy}benzoyl)glycine
3-(3,5-dichloro-4-{[3-(cyclobutylamino)-5-methylbenzyl]oxy}phenyl)propanoic acid
3-(3,5-dibromo-4-{[3-chloro-5-(cyclobutylamino)benzyl]oxy}phenyl)propanoic acid
N-(3,5-dibromo-4-{[3-chloro-5-(cyclobutylamino)benzyl]oxy benzoyl)glycine
3-(3,5-dibromo-4-([3-(cyclobutylamino)-5-(trifluoromethyl)benzyl]oxy}phenyl)propanoic acid
N-(3,5-dibromo-4-{[3-(cyclobutylamino)-5-(trifluoromethyl)benzyl]oxy}benzoyl)glycine
3-(3,5-dibromo-4-{[3-cyano-5-(cyclobutylamino)benzyl]oxy}phenyl)propanoic acid
N-(3,5-dibromo-4-([2-chloro-3-(cyclobutylamino)benzyl]oxy)benzoyl)glycine
3-(3,5-dibromo-4-{[2-chloro-3-(cyclobutylamino)benzyl]oxy}phenyl)propanoic acid
3-(3,5-dibromo-4-{[3-(isopropylamino)-5-methylbenzyl]oxy)phenyl)propanoic acid
N-(3,5-dibromo-4-([3-(isopropylamino)-5-methylbenzyl]oxy}benzoyl)glycine
3-(3,5-dichloro-4-{[3-(isopropylamino)-5-methylbenzyl]oxy}phenyl)propanoic acid
3-(3,5-dibromo-4-{[3-chloro-5-(isopropylamino)benzyl]oxy}phenyl)propanoic acid
N-(3,5-dibromo-4-{[3-chloro-5-(isopropylamino)benzyl]oxy}benzoyl)glycine
N-(3,5-dibromo-4-{[3-(isopropylamino)-5-(trifluoromethyl)benzyl]oxy}benzoyl)glycine
3-(3,5-dibromo-4-{[3-chloro-5-(isopropylamino)benzyl]oxy}phenyl)-2-fluoropropanoic acid
N-(3,5-dibromo-4-{[2-chloro-3-(isopropylamino)benzyl]oxy}benzoyl)glycine
3-(3,5-dibromo-4-{[2-chloro-3-(isopropylamino)benzyl]oxy}phenyl)propanoic acid
3-(3,5-dibromo-4-{[3-(sec-butylamino)-5-methylbenzyl]oxy}phenyl)propanoic acid
N-(3,5-dibromo-4-{[3-(sec-butylamino)-5-methylbenzyl]oxy}benzoyl)glycine
3-(4-{[3-(sec-butylamino)-5-methylbenzyl]oxy}-3,5-dichlorophenyl)propanoic acid
3-(3,5-dibromo-4-{[3-(sec-butylamino)-5-chlorobenzyl]oxy}phenyl)propanoic acid
N-(3,5-dibromo-4-{[3-(sec-butylamino)-5-chlorobenzyl]oxy}benzoyl)glycine
3-(3,5-dibromo-4-{[3-(sec-butylamino)-5-(trifluoromethyl)benzyl]oxy}phenyl)propanoic acid
N-(3,5-dibromo-4-{[3-(sec-butylamino)-5-(trifluoromethyl)benzyl]oxy}benzoyl)glycine
3-(3,5-dibromo-4-{[3-(sec-butylamino)-2-chlorobenzyl]oxy}phenyl)propanoic acid
(3,5-dibromo-4-{[3-chloro-5-(ethylamino)benzyl]oxy}phenyl)acetic acid
(3,5-dichloro-4-{[3-(ethylamino)-5-methyl-benzyl]oxy}phenyl)acetic acid
[4-(3-amino-5-methylbenzyloxy)-3,5-dichlorophenyl]acetic acid
N-(3,5-dichloro-4-{[3-(ethylamino)-5-methylbenzyl]oxy}benzoyl)glycine
N-{4-[(3-amino-5-methylbenzyl)oxy]-3,5-dichlorobenzoyl}glycine
3-(3,5-dibromo-4-{[3-(ethylamino)-5-(fluoromethyl)benzyl]oxy}phenyl)propanoic acid
3-(3,5-dibromo-4-{[3-ethoxymethyl-5-(ethylamino)benzyl]oxy}phenyl)propanoic acid
3-(4-[3-amino-2-chlorobenzyloxy]-3,5-bromophenyl) propanoic acid
3-(4-[3-amino-2-fluorobenzyloxy]-3,5-bromophenyl) propanoic acid
3-(3,5-dibromo-4-{[2-ethoxy-3-(ethylamino)benzyl]oxy}phenyl)propanoic acid
3-(3,5-dibromo-4-{[2-methoxy-3-(ethylamino)benzyl]oxy}phenyl)propanoic acid
3-(4-{[3-(2-carboxy-ethylamino)benzyl]oxy}-3,5-dichlorophenyl)propanoic acid
3-[3,5-dichloro-4-({3-[(cyclopropyl)amino]benzyl}oxy)phenyl]propanoic acid
3-(3,5-dibromo-4-{[2-fluoro-5-(isopropylamino)benzyl]oxy}phenyl)propanoic acid
3-(3,5-dibromo-4-{[2-fluoro-5-(cyclobutylamino)benzyl]oxy}phenyl)propanoic acid
3-(3,5-dibromo-4-{[2-fluoro-5-(sec-butylamino)benzyl]oxy}phenyl)propanoic acid
3-(3,5-dibromo-4-{[2-fluoro-5-(ethylamino)benzyl]oxy}phenyl)propanoic acid
3-(3,5-dibromo-4-{[2,5-dichloro-3-(ethylamino)benzyl]oxy}phenyl)propanoic acid
3-(3,5-dibromo-4-{[2,5-dichloro-3-(isopropylamino)benzyl]oxy}phenyl)propanoic acid
3-(3,5-dibromo-4-{[2,5-dichloro-3-(cyclobutylamino)benzyl]oxy}phenyl)propanoic acid
3-(3,5-dibromo-4-{[3-chloro-5-(1,2-dimethyl-propylamino)benzyl]oxy}phenyl)propanoic acid
3-(3,5-dibromo-4-{[2-chloro-3-(1,2-dimethyl-propylamino)benzyl]oxy}phenyl)propanoic acid
N-(3,5-dibromo-4-{[3-chloro-5-(1,2-dimethyl-propylamino)benzyl]oxy}benzoyl)glycine
3-(3,5-dibromo-4-{[2,5-dichloro-3-(isopropylamino)benzyl]oxy}phenyl)-2-fluoropropanoic acid
3-(3,5-dibromo-4-{[2,5-dichloro-3-(ethylamino)benzyl]oxy}phenyl)-2-fluoropropanoic acid
3-(3,5-dibromo-4-{[2-chloro-3-(ethylamino)benzyl]oxy}phenyl)-2-fluoropropanoic acid
3-(3,5-dibromo-4-{[3-(ethylamino)-5-methylbenzyl]oxy}phenyl)butanoic acid
3-(3,5-dibromo-4-{[3-(isopropylamino)-5-methylbenzyl]oxy}phenyl)butanoic acid
3-(3,5-dibromo-4-{[3-(cyclobutylamino)-5-methylbenzyl]oxy}phenyl)butanoic acid
3-(3,5-dibromo-4-{[2,5-dichloro-3-(ethylamino)benzyl]oxy}phenyl)butanoic acid
(E)-3-(3,5-dibromo-4-{[2,5-dichloro-3-(ethylamino)benzyl]oxy}phenyl)acrylic acid
(E)-3-(3,5-dibromo-4-{[2,5-dichloro-3-(isopropylamino)benzyl]oxy}phenyl)acrylic acid
(E)-3-(3,5-dibromo-4-{[2,5-dichloro-3-(cyclobutylamino)benzyl]oxy}phenyl)acrylic acid
N-(3,5-dibromo-4-{[2,5-dichloro-3-(ethylamino)benzyl]oxy}benzoyl)glycine
N-(3,5-dibromo-4-{[2,5-dichloro-3-(isopropylamino)benzyl]oxy}benzoyl)glycine
N-(3,5-dibromo-4-{[2,5-dichloro-3-(cyclobutylamino)benzyl]oxy}benzoyl)glycine
(S)-2-{2-(3,5-dibromo-4-{[3-chloro-5-(ethylamino)benzyl]oxy}phenyl)acetylamino}-3-phenyl-propanoic acid
(S)-2-{2-(3,5-dichloro-4-{[3-(ethylamino)-5-methylbenzyl]oxy}phenyl)acetylamino}-2-phenyl-acetic acid
3-[(3,5-dibromo-4-{[3-chloro-5-(isopropylamino)benzyl]oxy}phenyl)amino]-3-oxopropanoic acid
3-(3,5-dibromo-4-{[3-(ethylamino)-5-methylbenzyl]oxy}phenyl)-2-fluoropropanoic acid
3-(3,5-dibromo-4-{[3-(ethylamino)-2-fluoro-5-methylbenzyl]oxy}phenyl)propanoic acid
3-(3,5-dibromo-4-{[5-chloro-3-(ethylamino)-2-fluorobenzyl]oxy}phenyl)propanoic acid
3-[(3,5-dibromo-4-{[3-chloro-5-(methylamino)benzyl]oxy}phenyl)amino]-3-oxopropanoic acid
3-(3,5-dibromo-4-{[5-chloro-2-fluoro-3-(methylamino)benzyl]oxy}phenyl)propanoic acid
3-[3,5-dibromo-4-({3-chloro-5-[(cyclopropylmethyl)amino]benzyl}oxy)phenyl]propanoic acid
3-[3,5-dibromo-4-({3-[(cyclopropylmethyl)amino]-5-methylbenzyl}oxy)phenyl]-2-fluoropropanoic acid
3-{[3,5-dibromo-4-({3-[(cyclopropylmethyl)amino]-5-methylbenzyl}oxy)phenyl]amino}-3-oxopropanoic acid
3-[3,5-dibromo-4-({3-[(cyclopropylmethyl)amino]-2-fluoro-5-methylbenzyl}oxy)phenyl]propanoic acid
3-(3,5-dibromo-4-{[3-chloro-5-(cyclobutylamino)benzyl]oxy}phenyl)-2-fluoropropanoic acid
3-[(3,5-dibromo-4-{[3-(cyclobutylamino)-5-methylbenzyl]oxy}phenyl)amino]-3-oxopropanoic acid
3-(3,5-dibromo-4-{[3-(cyclobutylamino)-2-fluorobenzyl]oxy}phenyl)propanoic acid
3-(3,5-dibromo-4-{[3-(cyclobutylamino)-2-fluoro-5-methylbenzyl]oxy}phenyl)propanoic acid
3-(3,5-dibromo-4-{[5-chloro-3-(cyclobutylamino)-2-fluorobenzyl]oxy}phenyl)propanoic acid
3-(3,5-dibromo-4-{[3-(isopropylamino)-5-methylbenzyl]oxy}phenyl)-2-fluoropropanoic acid
3-[(3,5-dibromo-4-{[3-(isopropylamino)-5-methylbenzyl]oxy}phenyl)amino]-3-oxopropanoic acid
3-(3,5-dibromo-4-{[5-chloro-2-fluoro-3-(isopropylamino)benzyl]oxy}phenyl)propanoic acid
3-(3,5-dibromo-4-{[3-methyl-5-(propylamino)benzyl]oxy}phenyl)propanoic acid
3-(3,5-dibromo-4-{[5-chloro-2-fluoro-3-(propylamino)benzyl]oxy}phenyl)propanoic acid
3-(4-{[3-(sec-butylamino)-5-chlorobenzyl]oxy}-3,5-dichlorophenyl)-2-fluoropropanoic acid
3-[(3,5-dibromo-4-{[3-(sec-butylamino)-5-methylbenzyl]oxy}phenyl)amino]-3-oxopropanoic acid
3-(4-{[3-(sec-butylamino)-2-fluoro-5-methylbenzyl]oxy}-3,5-dichlorophenyl)propanoic acid
3-(3,5-dibromo-4-{[3-(sec-butylamino)-2-fluoro-5-methylbenzyl]oxy}phenyl)propanoic acid
3-(3,5-dibromo-4-{[3-(sec-butylamino)-5-chloro-2-fluorobenzyl]oxy}phenyl)propanoic acid
3-(3,5-dibromo-4-{[3-(cyclopropylamino)benzyl]oxy phenyl)propanoic acid
(4-[(E)-2-(3-amino-phenyl)-vinyl]-3,5-dibromo-benzyloxy}-acetic acid tert-butyl ester

Most particularly preferred compounds include:
3-(3,5-dibromo-4-{[3-(methylamino)benzyl]oxy}phenyl)propanoic acid
4-[(3-aminobenzyl)oxy]-3,5-dibromobenzoic acid
3-(4-{[3-amino-5-(trifluoromethyl)benzyl]oxy}-3,5-dibromophenyl)propanoic acid
3-(3,5-dibromo-4-{[3-(methylamino)-5-(trifluoromethyl)benzyl]oxy}phenyl)propanoic acid
3-(3,5-dibromo-4-{[2-methyl-3-(methylamino)benzyl]oxy}phenyl)propanoic acid
3-(3,5-dibromo-4-{[3-(ethylamino)-5-(trifluoromethyl)benzyl]oxy}phenyl)propanoic acid
3-(4-{[3-amino-5-chlorobenzyl)oxy]-3,5-dibromophenyl}propanoic acid
3-(3,5-dibromo-4-{[3-chloro-5-(ethylamino)benzyl]oxy}phenyl)propanoic acid
3-(4-{[3-amino-5-methylbenzyl)oxy]-3,5-dibromophenyl}propanoic acid
3-(3,5-dibromo-4-{[3-(ethylamino)-5-methylbenzyl]oxy}phenyl)propanoic acid
3-(4-[(3-amino-4-methylbenzyl)oxy]-3,5-dibromophenyl)propanoic acid
3-(3,5-dibromo-4-{[3-(ethylamino)-4-methylbenzyl]oxy}phenyl)propanoic acid
3-(3,5-dibromo-4-{[3-(ethylamino)-2-methylbenzyl]oxy}phenyl)propanoic acid
3-(3,5-dibromo-4-{[3-(propylamino)benzyl]oxy}phenyl)propanoic acid
3-(3,5-dibromo-4-{[2-methyl-3-(propylamino)benzyl]oxy}phenyl)propanoic acid
3-[3,5-dibromo-4-({3-[(2E)-but-2-en-1-ylamino]benzyl}oxy)phenyl]propanoic acid
3-[3,5-dibromo-4-({3-[(3,3-dimethylbutyl)amino]benzyl}oxy)phenyl]propanoic acid
3-{3,5-dibromo-4-[(3-{[2-(methylthio)ethyl]amino}benzyl)oxy]phenyl}propanoic acid
3-(3,5-dibromo-4-{[3-(isopropylamino)benzyl]oxy}phenyl)propanoic acid
3-(3,5-dibromo-4-{[3-(isopropylamino)-5-(trifluoromethyl)benzyl]oxy}phenyl)propanoic acid
3-(3,5-dibromo-4-{[3-(butylamino)benzyl]oxy}phenyl)propanoic acid
3-(3,5-dibromo-4-{[3-(sec-butylamino)benzyl]oxy}phenyl)propanoic acid
3-(3,5-dibromo-4-{[3-(sec-butylamino)-2-methylbenzyl]oxy}phenyl)propanoic acid
3-[3,5-dibromo-4-({3-[(2-ethylbutyl)amino]benzyl}oxy)phenyl]propanoic acid
3-(3,5-dibromo-4-{[3-(cyclobutylamino)benzyl]oxy}phenyl)propanoic acid
3-(3,5-dibromo-4-{[3-(cyclobutylamino)-2-methylbenzyl]oxy}phenyl)propanoic acid
3-[3,5-dibromo-4-({3-[(cyclopropylmethyl)amino]benzyl}oxy)phenyl]propanoic acid
3-[3,5-dibromo-4-({3-[(cyclohexylmethyl)amino]benzyl}oxy)phenyl]propanoic acid
3-(3,5-dibromo-4-([3-(isobutylamino)benzyl]oxy)phenyl)propanoic acid
3-{4-[(3-aminobenzyl)oxy]-3,5-dichlorophenyl}propanoic acid
3-(3,5-dichloro-4-{[3-(methylamino)benzyl]oxy}phenyl)propanoic acid
3-(3,5-dichloro-4-{[3-(ethylamino)benzyl]oxy}phenyl)propanoic acid
3-(3,5-dichloro-4-{[3-(ethylamino)-5-(trifluoromethyl)benzyl]oxy}phenyl)propanoic acid
3-(3,5-dichloro-4-{[3-(propylamino)benzyl]oxy}phenyl)propanoic acid
3-(3,5-dichloro-4-([3-(isopropylamino)benzyl]oxy phenyl)propanoic acid
3-(4-{[3-(sec-butylamino)benzyl]oxy}-3,5-dichlorophenyl)propanoic acid
3-[3,5-dichloro-4-{3-[(cyclopropylmethyl)amino]benzyl}oxy)phenyl]propanoie acid
N-(4-[(3-aminobenzyl)oxy]-3,5-dibromobenzoyl)glycine
N-(3,5-dibromo-4-{[3-(ethylamino)benzyl]oxy}benzoyl)glycine
N-(3,5-dibromo-4-{[3-(methylamino)benzyl]oxy}benzoyl)glycine
N-(3,5-dibromo-4-{[3-(propylamino)benzyl]oxy}benzoyl)glycine
N-(3,5-dibromo-4-{[3-(isopropylamino)benzyl]oxy}benzoyl)glycine
N-(3,5-dibromo-4-{[3-(cyclobutylamino)benzyl]oxy}benzoyl)glycine
N-(3,5-dibromo-4-{[3-(sec-butylamino)benzyl]oxy}benzoyl)glycine
N-(3,5-dichloro-4-{[3-(ethylamino)benzyl]oxy}benzoyl)glycine
N-(3,5-dibromo-4-{[3-(ethylamino)-5-(trifluoromethyl)benzyl]oxy}benzoyl)glycine
N-(3,5-dibromo-4-{[3-chloro-5-(ethylamino)benzyl]oxy}benzoyl)glycine
N-(3,5-dibromo-4-{[3-(ethylamino)-5-methylbenzyl]oxy}benzoyl)glycine
3-(3,5-dichloro-4-{[3-(ethylamino)-5-methylbenzyl]oxy}phenyl)propanoic acid
3-(3,5-dibromo-4-{[3-chloro-5-(ethylamino)benzyl]oxy}phenyl)-2-fluoropropanoic acid
(3,5-dichloro-4-{[3-(ethylamino)-5-methylbenzyl]oxy}phenyl)acetic acid
(3,5-dibromo-4-{[3-(ethylamino)benzyl]oxy}phenyl)acetic acid
3-(3,5-dibromo-4-{[3-cyano-5-(ethylamino)benzyl]oxy}phenyl)propanoic acid
3-(3,5-dibromo-4-{[3-(ethylamino)-2-fluorobenzyl]oxy}phenyl)propanoic acid
3-(3,5-dibromo-4-{[2-chloro-3-(ethylamino)benzyl]oxy}phenyl)propanoic acid
N-(3,5-dibromo-4-{[2-chloro-3-(ethylamino)benzyl]oxy}benzoyl)glycine
3-(3,5-dibromo-4-{[3-chloro-5-(methylamino)benzyl]oxy}phenyl)propanoic acid
3-(3,5-dibromo-4-{[3-methyl-5-(methylamino)benzyl]oxy}phenyl)propanoic acid
3-(3,5-dibromo-4-{[3-(cyclobutylamino)-5-methylbenzyl]oxy}phenyl)propanoic acid
N-(3,5-dibromo-4-{[3-(cyclobutylamino)-5-methylbenzyl]oxy}benzoyl)glycine
3-(3,5-dichloro-4-{[3-(cyclobutylamino)-5-methylbenzyl]oxy}phenyl)propanoic acid
3-(3,5-dibromo-4-{[3-chloro-5-(cyclobutylamino)benzyl]oxy}phenyl)propanoic acid
N-(3,5-dibromo-4-{[3-chloro-5-(cyclobutylamino)benzyl]oxy}benzoyl)glycine
3-(3,5-dibromo-4-{[3-(cyclobutylamino)-5-(trifluoromethyl)benzyl}oxy)phenyl]propanoic acid
N-(3,5-dibromo-4-{[3-(cyclobutylamino)-5-(trifluoromethyl)benzyl]oxy}benzoyl)glycine
3-(3,5-dibromo-4-{[3-cyano-5-(cyclobutylamino)benzyl]oxy}phenyl)propanoic acid
N-(3,5-dibromo-4-{[2-chloro-3-(cyclobutylamino)benzyl]oxy}benzoyl)glycine
3-(3,5-dibromo-4-{[2-chloro-3-(cyclobutylamino)benzyl]oxy}phenyl)propanoic acid
3-(3,5-dibromo-4-{[3-(isopropylamino)-5-methylbenzyl]oxy}phenyl)propanoic acid
N-(3,5-dibromo-4-{[3-(isopropylamino)-5-methylbenzyl]oxy}benzoyl)glycine
3-(3,5-dichloro-4-{[3-(isopropylamino)-5-methylbenzyl]oxy}phenyl)propanoic acid
3-(3,5-dibromo-4-{[3-chloro-5-(isopropylamino)benzyl]oxy}phenyl)propanoic acid
N-(3,5-dibromo-4-{[3-chloro-5-(isopropylamino)benzyl]oxy}benzoyl)glycine
N-(3,5-dibromo-4-{[3-(isopropylamino)-5-(trifluoromethyl)benzyl]oxy}benzoyl)glycine
3-(3,5-dibromo-4-{[3-chloro-5-(isopropylamino)benzyl]oxy}phenyl)-2-fluoropropanoic acid
N-(3,5-dibromo-4-{[2-chloro-3-(isopropylamino)benzyl]oxy}benzoyl)glycine
3-(3,5-dibromo-4-{[2-chloro-3-(isopropylamino)benzyl]oxy}phenyl)propanoic acid
3-(3,5-dibromo-4-{[3-(sec-butylamino)-5-methylbenzyl]oxy}phenyl)propanoic acid
N-(3,5-dibromo-4-{[3-(sec-butylamino)-5-methylbenzyl]oxy}benzoyl)glycine
3-(4-{[3-(sec-butylamino)-5-methylbenzyl]oxy}-3,5-dichlorophenyl)propanoic acid
3-(3,5-dibromo-4-{[3-(sec-butylamino)-5-chlorobenzyl]oxy}phenyl)propanoic acid
N-(3,5-dibromo-4-{[3-(sec-butylamino)-5-chlorobenzyl]oxy}benzoyl)glycine
3-(3,5-dibromo-4-{[3-(sec-butylamino)-5-(trifluoromethyl)benzyl]oxy}phenyl)propanoic acid
N-(3,5-dibromo-4-{[3-(sec-butylamino)-5-(trifluoromethyl)benzyl]oxy}benzoyl)glycine
3-(3,5-dibromo-4-{[3-(sec-butylamino)-2-chlorobenzyl]oxy}phenyl)propanoic acid
(3,5-dibromo-4-{[3-chloro-5-(ethylamino)benzyl]oxy}phenyl)acetic acid
(3,5-dichloro-4-{[3-(ethylamino)-5-methyl-benzyl]oxy}phenyl)acetic acid
[4-(3-amino-5-methylbenzyloxy)-3,5-dichlorophenyl]acetic acid
N-(3,5-dichloro-4-{[3-(ethylamino)-5-methylbenzyl]oxy}benzoyl)glycine
N-{4-[(3-amino-5-methylbenzyl)oxy]-3,5-dichlorobenzoyl}glycine
3-(3,5-dibromo-4-{[3-(ethylamino)-5-(fluoromethyl)benzyl]oxy}phenyl)propanoic acid
3-(3,5-dibromo-4-{[3-ethoxymethyl-5-(ethylamino)benzyl]oxy}phenyl)propanoic acid
3-(4-[3-amino-2-chlorobenzyloxy]-3,5-bromophenyl)propanoic acid
3-(4-[3-amino-2-fluorobenzyloxy]-3,5-bromophenyl)propanoic acid
3-(3,5-dibromo-4-{[2-ethoxy-3-(ethylamino)benzyl]oxy}phenyl)propanoic acid
3-(3,5-dibromo-4-{[2-methoxy-3-(ethylamino)benzyl]oxy}phenyl)propanoic acid
3-(4-{[3-(2-carboxy-ethylamino)benzyl]oxy}-3,5-dichlorophenyl)propanoic acid
3-[3,5-dichloro-4-({3-[(cyclopropyl)amino]benzyl}oxy)phenyl]propanoic acid
3-(3,5-dibromo-4-{[2-fluoro-5-(isopropylamino)benzyl]oxy}phenyl)propanoic acid
3-(3,5-dibromo-4-{[2-fluoro-5-(cyclobutylamino)benzyl]oxy}phenyl)propanoic acid
3-(3,5-dibromo-4-{[2-fluoro-5-(sec-butylamino)benzyl]oxy}phenyl)propanoic acid
3-(3,5-dibromo-4-{[2-fluoro-5-(ethylamino)benzyl]oxy}phenyl)propanoic acid
3-(3,5-dibromo-4-{[2,5-dichloro-3-(ethylamino)benzyl]oxy}phenyl)propanoic acid
3-(3,5-dibromo-4-{[2,5-dichloro-3-(isopropylamino)benzyl]oxy}phenyl)propanoic acid
3-(3,5-dibromo-4-{[2,5-dichloro-3-(cyclobutylamino)benzyl]oxy}phenyl)propanoic acid
3-(3,5-dibromo-4-{[3-chloro-5-(1,2-dimethyl-propylamino)benzyl]oxy}phenyl)propanoic acid
3-(3,5-dibromo-4-{[2-chloro-3-(1,2-dimethyl-propylamino)benzyl]oxy}phenyl)propanoic acid
N-(3,5-dibromo-4-{[3-chloro-5-(1,2-dimethyl-propylamino)benzyl]oxy}benzoyl)glycine
3-(3,5-dibromo-4-{[2,5-dichloro-3-(isopropylamino)benzyl]oxy}phenyl)-2-fluoropropanoic acid
3-(3,5-dibromo-4-{[2,5-dichloro-3-(ethylamino)benzyl]oxy}phenyl)-2-fluoropropanoic acid
3-(3,5-dibromo-4-{[2-chloro-3-(ethylamino)benzyl]oxy}phenyl)-2-fluoropropanoic acid
3-(3,5-dibromo-4-{[3-(ethylamino)-5-methylbenzyl]oxy}phenyl)butanoic acid
3-(3,5-dibromo-4-{[3-(isopropylamino)-5-methylbenzyl]oxy}phenyl)butanoic acid
3-(3,5-dibromo-4-{[3-(cyclobutylamino)-5-methylbenzyl]oxy}phenyl)butanoic acid
3-(3,5-dibromo-4-{[2,5-dichloro-3-(ethylamino)benzyl]oxy}phenyl)butanoic acid
(E)-3-(3,5-dibromo-4-{[2,5-dichloro-3-(ethylamino)benzyl]oxy}phenyl)acrylic acid
(E)-3-(3,5-dibromo-4-{[2,5-dichloro-3-(isopropylamino)benzyl]oxy}phenyl)acrylic acid
(E)-3-(3,5-dibromo-4-{[2,5-dichloro-3-(cyclobutylamino)benzyl]oxy}phenyl)acrylic acid
N-(3,5-dibromo-4-{[2,5-dichloro-3-(ethylamino)benzyl]oxy}benzoyl)glycine
N-(3,5-dibromo-4-{[2,5-dichloro-3-(isopropylamino)benzyl]oxy}benzoyl)glycine
N-(3,5-dibromo-4-{[2,5-dichloro-3-(cyclobutylamino)benzyl]oxy}benzoyl)glycine
(S)-2-{2-(3,5-dibromo-4-{[3-chloro-5-(ethylamino)benzyl]oxy}phenyl)acetylamino}-3-phenyl-propanoic acid
(S)-2-{2-(3,5-dichloro-4-{[3-(ethylamino)-5-methylbenzyl]oxy}phenyl)acetylamino}-2-phenyl-acetic acid
3-[(3,5-dibromo-4-{[3-chloro-5-(isopropylamino)benzyl]oxy}phenyl)amino]-3-oxopropanoic acid
{4-[(E)-2-(3-amino-phenyl)-vinyl]-3,5-dibromo-benzyloxy}-acetic acid tert-butyl ester

The compounds names given above were generated in accordance with IUPAC by the ACD Labs/Name program, version 7.08 build 21 and with ISIS DRAW Autonom 2000.

Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. According to the present invention, examples of physiologically functional derivatives include esters, amides, and carbamates; preferably esters and amides.

Suitable salts according to the invention include those formed with organic or inorganic acids or bases. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycollic, lactic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic, while not in themselves pharmaceutically acceptable, may be useful as intermediates in obtaining the compounds of the invention and their pharmaceutical acceptable acid addition salts. Pharmaceutically acceptable base salts include ammonium salts, alkali metal salts, for example those of potassium and sodium, alkaline earth metal salts, for example those of calcium and magnesium, and salts with organic bases e.g. primary, secondary or tertiary organic amines, for example dicyclohexylamine, and N-methyl-D-glucomine.

Pharmaceutically acceptable esters and amides of the compounds of formula (I) may have an appropriate group, for example an acid group, converted to a C₁₋₆ alkyl, C₅₋₁₀ aryl, C₅₋₁₀ ar-C₁₋₆ alkyl, or amino acid ester or amide. Pharmaceutically acceptable amides and carbonates of the compounds of formula (I) may have an appropriate group, for example an amino group, converted to a C₁₋₆ alkyl, C₅₋₁₀ aryl, C₅₋₁₀ aryl-C₁₋₆ alkyl, or amino acid ester or amide, or carbamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above or an active metabolite or residue thereof, is known as a “prodrug”. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); and in Edward B. Roche, ed., Bioreversible Carriers in Drug Design, American Pharmaceutical Association and Pergamon Press, 1987, both of which are incorporated herein by reference.

As used herein, the term “alkyl” means both straight and branched chain saturated hydrocarbon groups. Examples of alkyl groups include methyl, ethyl, n-propyl, iso-propyl, n-butyl, sec-butyl, t-butyl, i-butyl, pentyl, hexyl, heptyl, octyl, nonyl and decyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl, 1-ethyl butyl and 1-ethylpentyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Up to 5 carbon carbon double bonds may, for example, be present. Examples of alkenyl groups include ethenyl, propenyl, butenyl, pentenyl, hexenyl, heptenyl, octenyl, nonenyl, decenyl and dodecenyl. Preferred alkenyl groups includes ethenyl, 1-propenyl and 2-propenyl.

As used herein, the term “alkenyloxy” means the group O-alkenyl, where “alkenyl” is used as described above. Examples of alkenyloxy groups include ethenyloxy groups. Other examples include 2-propenyloxy, 3-butenyloxy and 4-pentenyloxy.

As used herein, the term “alkynyl” means both straight and branched chain unsaturated hydrocarbon groups with at least one carbon carbon triple bond. Up to 5 carbon carbon triple bonds may, for example, be present. Examples of alkynyl groups include ethynyl, propynyl, butynyl, pentynyl, hexynyl, heptynyl, octynyl, nonynyl, decynyl and dodecynyl. Preferred alkenyl groups include ethynyl 1-propynyl and 2-propynyl.

As used herein, the term “cycloalkyl” means a saturated group in a ring system. The cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo [2.2.1]hept-2-yl. Preferably, the cycloalkyl group is monocyclic.

As used herein, the term “cycloalkenyl” means an unsaturated aliphatic group in a ring system. A cycloalkenyl group can be monocyclic or bicyclic. Preferably, the cycloalkyl group is monocyclic. Examples of monocyclic cycloalkenyl groups include cyclopentenyl and cyclohexenyl.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “aryloxy” means the group O-aryl, where “aryl” is used as described above.

As used herein, the term “halogen” means fluorine, chlorine, bromine or iodine. Fluorine, chlorine and bromine are particularly preferred.

As used herein, the term “heterocyclyl” means an aromatic (“heteroaryl”) or a non-aromatic (“heterocycloalkyl”) cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O or N. Heterocyclyl groups containing a suitable nitrogen atom include the corresponding N-oxides. Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

Examples of preferred heterocyclyl groups include piperidinyl, tetrahydrofuranyl, tetrahydropyranyl, pyridyl, pyrimidyl and indolyl.

As used herein, the term “arylalkyl” means a group aryl-alkyl-attached through the alkyl group, “aryl” and “alkyl” being understood to have the meanings outlined above.

As used herein the term “cycloalkylalkyl” means a group cycloalkyl-alkyl-attached through the alkyl group, “cycloalkyl” and “alkyl” being understood to have the meanings outlined above.

As used herein the term “cycloalkylalkoxy” means a group cycloalkyl-alkoxy-attached through the alkoxy group, “cycloalkyl” and “alkoxy” being understood to have the meanings outlined above.

As used herein the term “arylalkoxy” means a group aryl-alkoxy-attached through the alkoxy group, “aryl” and “alkoxy” being understood to have the meanings outlined above.

As used herein, the term “heterocyclylalkyl” means a group heterocyclyl-alkyl-attached through the alkyl group, “heterocyclyl” and “alkyl” being understood to have the meanings outlined above. Similarly, as used herein, the term “heterocyclylalkenyl” means a group heterocyclyl-alkenyl-attached through the alkenyl group, “heterocyclyl” and “alkenyl” being understood to have the meanings outlined above.

As mentioned above, the compounds of the invention have activity as thyroid receptor ligands. The compounds of the invention are preferably selective agonists or partial agonists of the thyroid receptor. Preferably compounds of the present invention possess activity as agonists of the thyroid receptor, preferably selective agonists of the thyroid receptor-beta. They may thus be used in the treatment of diseases or disorders associated with thyroid receptor activity. In particular, compounds of the present invention may be used in the treatment of diseases or disorders associated with metabolism dysfunction or which are dependent upon the expression of a T₃ regulated gene. The invention accordingly provides a compound of formula (I) as defined above or a pharmaceutically acceptable ester, amide, solvate or salt thereof, including a salt of such an ester or amide, and a solvate of such an ester, amide or salt for use in such treatments.

Clinical conditions for which an agonist or partial agonist is indicated include, but are not limited to, hypothyroidism; subclinical hyperthyroidism; non-toxic goiter; atherosclerosis; thyroid hormone replacement therapy (e.g., in the elderly); malignant tumor cells containing the thyroid receptor; papillary or follicular cancer; maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); accelerating healing of complicated fractures (e.g. distraction osteogenesis); in joint replacement; eating disorders (e.g., anorexia); treatment of obesity and growth retardation associated with obesity; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of hyperinsulinemia; stimulation of osteoblasts, bone remodeling and cartilage growth; regulation of food intake; treatment of insulin resistance, including NIDDM, in mammals (e.g., humans); treatment of insulin resistance in the heart; treatment of congestive heart failure; treatment of musculoskeletal impairment (e.g., in the elderly); improvement of the overall pulmonary function; skin disorders or diseases, such as dermal atrophy, glucocorticoid induced dermal atrophy, including restoration of dermal atrophy induced by topical glucocorticoids, and the prevention of dermal atrophy induced by topical glucocorticoids (such as the simultaneous treatment with topical glucocorticoid or a pharmacological product including both glucocorticoid and a compound of the invention), the restoration/prevention of dermal atrophy induced by systemic treatment with glucocorticoids, restoration/prevention of atrophy in the respiratory system induced by local treatment with glucocorticoids, UV-induced dermal atrophy, dermal atrophy induced by aging (wrinkles, etc.), wound healing, post surgical bruising caused by laser resurfacing, keloids, stria, cellulite, roughened skin, actinic skin damage, lichen planus, ichtyosis, acne, psoriasis, Dernier's disease, eczema, atopic dermatitis, chloracne, pityriasis and skin scarring. In addition, the conditions, diseases, and maladies collectively referenced to as “Syndrome X” or Metabolic Syndrome as detailed in Johannsson J. Clin. Endocrinol. Metab., 82, 727-34 (1997), may be treated employing the compounds of the invention. The term treatment includes, where appropriate, prophylactic treatment.

The compounds of the invention find particular application in the treatment or prophylaxis of the following: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

The compounds of the invention find especial application in the treatment or prophylaxis of the following: (1) hypercholesterolemia, dyslipidemia or any other lipid disorder manifested by an unbalance of blood or tissue lipid levels; (2) atherosclerosis; (3) obesity; (4) diabetes.

The invention also provides a method for the treatment or prophylaxis of a condition in a mammal mediated by a thyroid receptor, which comprises administering to the mammal a therapeutically effective amount of a compound of formula (I) as defined above or a pharmaceutically acceptable ester, amide, solvate or salt thereof, including a salt of such an ester or amide, and a solvate of such an ester, amide or salt. Clinical conditions mediated by a thyroid receptor that may be treated by the method of the invention are those described above.

The invention also provides the use of a compound of formula (I) as defined above or a pharmaceutically acceptable ester, amide, solvate or salt thereof, including a salt of such an ester or amide, and a solvate of such an ester, amide or salt, for the manufacture of a medicament for the treatment or prophylaxis of a condition mediated by a thyroid receptor. Clinical conditions mediated by a thyroid receptor that may be treated by the method of the invention are those described above.

Hereinafter, the term “active ingredient” means a compound of formula (I) as defined above, or a pharmaceutically acceptable ester, amide, solvate or salt thereof, including a salt of such an ester or amide, and a solvate of such an ester, amide or salt.

The amount of active ingredient which is required to achieve a therapeutic effect will, of course, vary with the particular compound, the route of administration, the subject under treatment, and the particular disorder or disease being treated. The compounds of the invention may be administered orally or via injection at a dose of from 0.001 to 1500 mg/kg per day, preferably from 0.01 to 1500 mg/kg per day, more preferably from 0.1 to 1500 mg/kg per day, most preferably 0.1 to 500 mg/kg per day. The dose range for adult humans is generally from 5 mg to 35 g per day and preferably 5 mg to 2 g per day. Tablets or other forms of presentation provided in discrete units may conveniently contain an amount of compound of the invention which is effective at such dosage or as a multiple of the same, for example units containing 5 mg to 500 mg, usually around 10 mg to 200 mg.

While it is possible for the active ingredient to be administered alone, it is preferable for it to be present in a pharmaceutical formulation. Accordingly, the invention provides a pharmaceutical formulation comprising a compound of formula (I) as defined above or a pharmaceutically acceptable ester, amide, solvate or salt thereof, including a salt of such an ester or amide, and a solvate of such an ester, amide or salt, and a pharmaceutically acceptable excipient.

The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered does pressurized aerosols), nebulizers or insufflators, rectal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of formula I can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquify and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further active agents. Such further active agents may be further compounds according to the invention, or they may be different therapeutic agents, for example an anti-dyslipidemic agent or other pharmaceutically active material.

The compounds of the present invention may be employed in combination with one or more other modulators and/or ligands of the thyroid receptor or one or more other suitable therapeutic agents selected from the group consisting of cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

Examples of suitable hypolipidemic agents for use in combination with the compounds of the present invention include an acyl coenzyme A cholesterol acyltransferase (ACAT) inhibitor, a microsomal triglyceride transfer protein (MTP) inhibitor, a cholesterol ester transfer protein (CETP) inhibitor, a ileal bile acid transporter (IBAT) inhibitor, any cholesterol absorption inhibitor, a 3-hydroxy-3-methylglutaryl coenzyme A (HMG-CoA) reductase inhibitor, a squalene synthetase inhibitor, a bile acid sequestrant, a peroxisome proliferator-activator receptor (PPAR)-alpha agonist, a peroxisome proliferator-activator receptor (PPAR)-delta agonist, any peroxisome proliferator-activator receptor (PPAR)-gamma/delta dual agonist, any peroxisome proliferator-activator receptor (PPAR)-alpha/delta dual agonist, a nicotinic acid or a derivative thereof, and a thiazolidinedione or a derivative thereof.

Examples of suitable hypolipidemic agents for use in combination with the compounds of the present invention also include ezetimibe, simvastatin, atorvastatin, rosuvastatin, cerivastatin, fluvastatin, lovastatin, pravastatin, fenofibrate, gemfibrozil and bezafibrate.

Examples of suitable anti-diabetic agents for use in combination with the compounds of the present invention include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, glipyride, gliclazide, chlorpropamide and glipizide), biguanide/glyburide combinations (e.g., Glucovance®), thiazolidinediones (e.g., troglitazone, rosiglitazone, englitazone, darglitazone and pioglitazone), PPAR-alpha agonists, PPAR-gamma agonists, PPAR alpha/gamma dual agonists, PPAR alpha/delta dual agonists, SGLT 1, 2 or 3 inhibitors, glycogen phosphorylase inhibitors, inhibitors of fatty acid binding protein (aP2), glucagon-like peptide-1 (GLP-1), glucocorticoid (GR) antagonist and dipeptidyl peptidase IV (DP4) inhibitors.

Examples of suitable anti-osteoporosis agents for use in combination with the compounds of the present invention include alendronate, risedronate, PTH, PTH fragment, raloxifene, calcitonin, RANK ligand antagonists, calcium sensing receptor antagonists, TRAP inhibitors, selective estrogen receptor modulators (SERM) and AP-1 inhibitors.

Examples of suitable anti-obesity agents for use in combination with the compounds of the present invention include aP2 inhibitors, PPAR gamma antagonists, PPAR delta agonists, beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), other thyroid receptor beta drugs, such as a thyroid receptor ligand as disclosed in WO 97/21993 (U. Cal SF), WO 99/00353 (KaroBio) and GB98/284425 (KaroBio), CB-1 (cannabinoid receptor) antagonists (see G. Colombo et al, “Appetite Suppression and Weight Loss After the Cannabionid Antagonist SR 141716”, Life Sciences, Vol 63, PL 113-117 (1998)) and/or an anorectic agent, such as dexamphetamine, phentermine, phenylpropanolamine or mazindol.

The compounds of the present invention may be combined with growth promoting agents, such as, but not limited to, TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890.

The compounds of the invention may also be used in combination with growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or with growth hormone releasing factor and its analogs or growth hormone and its analogs or somatomedins including IGF-1 and IGF-2, or with alpha-adrenergic agonists, such as clonidine or serotinin 5-HT_{D} agonists, such as sumatriptan, or agents which inhibit somatostatin or its release, such as physostigmine and pyridostigmine. A still further use of the disclosed compounds of the invention is in combination with parathyroid hormone, PTH(1-34) or bisphosphonates, such as MK-217 (alendronate).

Examples of suitable anti-inflammatory agents for use in combination with the compounds of the present invention include prednisone, dexamethasone, Enbrel®, cyclooxygenase inhibitors (i.e., COX-1 and/or COX-2 inhibitors such as NSAIDs, aspirin, indomethacin, ibuprofen, piroxicam, Naproxen®, Celebrex®, Vioxx®), CTLA4-Ig agonists/antagonists, CD40 ligand antagonists, IMPDH inhibitors, such as mycophenolate (CellCept®), integrin antagonists, alpha-4 beta-7 integrin antagonists, cell adhesion inhibitors, interferon gamma antagonists, ICAM-1, tumor necrosis factor (TNF) antagonists (e.g., infliximab, OR 1384), prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, and therapies for the treatment of irritable bowel syndrome (e.g., Zelmac® and Maxi-K® openers such as those disclosed in U.S. Pat. No. 6,184,231 B1).

Example of suitable anti-anxiety agents for use in combination with the compounds of the present invention include diazepam, lorazepam, buspirone, oxazepam, and hydroxyzine pamoate.

Examples of suitable anti-depressants for use in combination with the compounds of the present invention include citalopram, fluoxetine, nefazodone, sertraline, and paroxetine.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable cardiac glycosides for use in combination with the compounds of the present invention include digitalis and ouabain.

Examples of suitable cholesterol/lipid lowering agents for use in combination with the compounds of the present invention include HMG-CoA reductase inhibitors, squalene synthetase inhibitors, fibrates, bile acid sequestrants, ACAT inhibitors, MTP inhibitors, lipooxygenase inhibitors, an ileal Na⁺/bile acid cotransporter inhibitor, cholesterol absorption inhibitors, and cholesterol ester transfer protein inhibitors (e.g., CP-529414).

MTP inhibitors which may be employed herein in combination with one or more compounds of formula I include MTP inhibitors as disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440 all incorporated herein by reference.

The HMG CoA reductase inhibitors which may be employed in combination with one or more compounds of formula I include mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Further HMG CoA reductase inhibitors which may be employed herein include fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pyrazole analogs of mevalonolactone derivatives as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No. 0,142,146 A2, as well as other known HMG CoA reductase inhibitors.

The squalene synthetase inhibitors which may be used in combination with the compounds of the present invention include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinylmethyl)phosphonates, terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, as well as other squalene synthetase inhibitors as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996).

Bile acid sequestrants which may be used in combination with the compounds of the present invention include cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives such as disclosed in U.S. Pat. No. 4,759,923, quaternary amine poly(diallyidimethylammonium chloride) and ionenes such as disclosed in U.S. Pat. No. 4,027,009, and other known serum cholesterol lowering agents.

ACAT inhibitors suitable for use in combination with compounds of the invention include ACAT inhibitors as described in, Drugs of the Future 24, 9-15 (1999), (Avasimibe); “The ACAT inhibitor, CI-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62.

Examples of suitable cholesterol absorption inhibitor for use in combination with the compounds of the invention include SCH48461 (Schering-Plough), as well as those disclosed in Atherosclerosis 115, 45-63 (1995) and J. Med. Chem. 41, 973 (1998).

Examples of suitable ileal Na⁺/bile acid cotransporter inhibitors for use in combination with the compounds of the invention include compounds as disclosed in Drugs of the Future, 24, 425-430 (1999).

Examples of suitable thyroid mimetics for use in combination with the compounds of the present invention include thyrotropin, polythyroid, KB-130015, and dronedarone.

Examples of suitable anabolic agents for use in combination with the compounds of the present invention include testosterone, TRH diethylstilbesterol, estrogens, β-agonists, theophylline, anabolic steroids, dehydroepiandrosterone, enkephalins, E-series prostagladins, retinoic acid and compounds as disclosed in U.S. Pat. No. 3,239,345, e.g., Zeranol®; U.S. Pat. No. 4,036,979, e.g., Sulbenox® or peptides as disclosed in U.S. Pat. No. 4,411,890.

For the treatment of skin disorders or diseases as described above, the compounds of the present invention may be used alone or optionally in combination with a retinoid, such as tretinoin, or a vitamin D analog.

A still further use of the compounds of the invention is in combination with estrogen, testosterone, a selective estrogen receptor modulator, such as tamoxifen or raloxifene, or other androgen receptor modulators, such as those disclosed in Edwards, J. P. et al., Bio. Med. Chem. Let., 9, 1003-1008 (1999) and Hamann, L. G. et al., J. Med. Chem., 42, 210-212 (1999).

A further use of the compounds of this invention is in combination with steriodal or non-steroidal progesterone receptor agonists (“PRA”), such as levonorgestrel, medroxyprogesterone acetate (MPA).

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with a hypolypidemic agent, an antidepressant, a bone resorption inhibitor and/or an appetite suppressant, the compounds of formula I may be employed in a weight ratio to the additional agent within the range from about 500:1 to about 0.005:1, preferably from about 300:1 to about 0.01:1.

Where the antidiabetic agent is a biguanide, the compounds of formula I may be employed in a weight ratio to biguanide within the range from about 0.01:1 to about 100:1, preferably from about 0.5:1 to about 2:1.

The compounds of formula I may be employed in a weight ratio to a glucosidase inhibitor within the range from about 0.01:1 to about 100:1, preferably from about 0.5:1 to about 50:1.

The compounds of formula I may be employed in a weight ratio to a sulfonylurea in the range from about 0.01:1 to about 100:1, preferably from about 0.2:1 to about 10:1.

The compounds of formula I may be employed in a weight ratio to a thiazolidinedione in an amount within the range from about 0.01:1 to about 100:1, preferably from about 0.5:1 to about 5:1. The thiazolidinedione may be employed in amounts within the range from about 0.01 to about 2000 mg/day, which may optionally be administered in single or divided doses of one to four times per day. Further, where the sulfonylurea and thiazolidinedione are to be administered orally in an amount of less than about 150 mg, these additional agents may be incorporated into a combined single tablet with a therapeutically effective amount of the compounds of formula I.

Metformin, or salt thereof, may be employed with the compounds of formula I in amounts within the range from about 500 to about 2000 mg per day, which may be administered in single or divided doses one to four times daily.

The compounds of formula I may be employed in a weight ratio to a PPAR-alpha agonist, a PPAR-gamma agonist, a PPAR-alpha/gamma dual agonist, an SGLT2 inhibitor and/or an aP2 inhibitor within the range from about 0.01:1 to about 100:1, preferably from about 0.5:1 to about 5:1.

An MTP inhibitor may be administered orally with the compounds of formula I in an amount within the range of from about 0.01 mg/kg to about 100 mg/kg and preferably from about 0.1 mg/kg to about 75 mg/kg, one to four times daily. A preferred oral dosage form, such as tablets or capsules, may contain the MTP inhibitor in an amount of from about 1 to about 500 mg, preferably from about 2 to about 400 mg, and more preferably from about 5 to about 250 mg, administered on a regimen of one to four times daily. For parenteral administration, the MTP inhibitor may be employed in an amount within the range of from about 0.005 mg/kg to about 10 mg/kg and preferably from about 0.005 mg/kg to about 8 mg/kg, administered on a regimen of one to four times daily.

A HMG CoA reductase inhibitor may be administered orally with the compounds of formula I within the range of from about 1 to 2000 mg, and preferably from about 4 to about 200 mg. A preferred oral dosage form, such as tablets or capsules, will contain the HMG CoA reductase inhibitor in an amount from about 0.1 to about 100 mg, preferably from about 5 to about 80 mg, and more preferably from about 10 to about 40 mg.

A squalene synthetase inhibitor may be administered with the compounds of formula I within the range of from about 10 mg to about 2000 mg and preferably from about 25 mg to about 200 mg. A preferred oral dosage form, such as tablets or capsules, will contain the squalene synthetase inhibitor in an amount of from about 10 to about 500 mg, preferably from about 25 to about 200 mg.

The compounds of formula (I) as described above also find use, optionally in labelled form, as a diagnostic agent for the diagnosis of conditions associated with malfunction of the thyroid receptor. For example, such a compound may be radioactively labelled.

The compounds of formula (I) as described above, optionally in labelled form, also find use as a reference compound in methods of discovering other antagonists or partial antagonists of the thyroid receptor. Thus, the invention provides a method of discovering a ligand of the thyroid receptor which comprises use of a compound of the invention or a compound of the invention in labelled form, as a reference compound. For example, such a method nay involve a competitive binding experiment in which binding of a compound of formula (I) to the thyroid receptor is reduced by the presence of a further compound which has thyroid receptor-binding characteristics, for example stronger thyroid receptor-binding characteristics than the compound of formula (I) in question.

The invention further provides a method for preparing a compound of formula (I) as defined above in which R¹ is not H, comprising a step of reacting
a compound of formula (II)

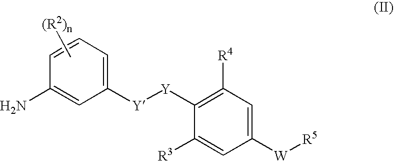

wherein R², n, Y′, Y, R³, R⁴, W and R⁵ are as defined in claim 1
with a compound of formula R^{1′}—CHO or R^{1″}—C(O)—R^{1′″}, wherein R^{1′}, R^{1″} and R^{1′″} are chosen such that the product compound comprises the group R¹ as defined above, optionally in the presence of a suitable reducing agent, followed optionally by interconversion to another compound as defined above.

Suitable reducing agents include sodium cyanoborohydride.

The invention also provides a method for preparing a compound of formula (I) as defined above in which R¹ is H, comprising a step of reacting
a compound of formula (III)

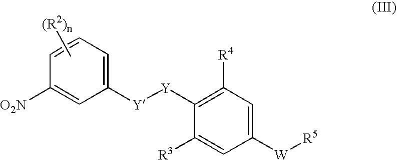

wherein R², n, Y′, Y, R³, R⁴, W and R⁵ are as defined in claim 1
with a suitable reducing agent, followed optionally by interconversion to another compound as defined in claim 1.

Suitable reducing agents include tin(II) chloride.

The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The invention will now be illustrated by the following Examples, which do not in any way limit the scope of the invention.

### EXAMPLES

The following compounds illustrate compounds of the invention or, where appropriate, compounds for use in the invention.

### Description 1

### Methyl 3-(4-hydroxy-3,5-dibromophenyl)propionate

To a solution of 3-(4-hydroxyphenyl)propionate methyl ester (10 g, 55.5 mmol) in acetic acid (150 mL), bromine (19.5 g, 121.9 mmol) was added drop wise slowly. The reaction mixture was stirred for 5 h at room temperature and then evaporated and co-evaporated with ethyl acetate (2×200 mL). The residue was purified on silica gel column to give 17.0 g of the title compound (90.6% yield).

### Description 2

### Methyl 3-(4-hydroxy-3,5-dichlorophenyl) propionate

Methyl-3-(4-hydroxyphenyl) propionate (35.6 g, 0.198 mol) was dissolved in dichloromethane (200 mL). The reaction mixture was cooled to 4° C. and sulfuryl chloride (120 mL, 1.42 mmol) in diethyl ether (200 mL) was added drop wise to the reaction mixture over 1 h. After 3 h at room temperature the solvent was removed. The reaction mixture was dissolved in dichloromethane and washed with water. The combined organic phases were dried over sodium sulphate, filtered and evaporated. The product was purified by flash chromatography (diethyl ether/heptane) to provide 16.6 g (34%) of the title compound.

### Description 3

### Methyl 4-(4-hydroxy-3,5-dibromophenyl) butanoate

To methyl 4-(4-hydroxyphenyl)butanoate (0.315 g, 1.6 mmol) dissolved in 1:1 methanol/dichloromethane solution (15 mL) at 0° C. was added BnNMe₃⁺Br₃⁻ (1.26 g, 3.2 mmol) and calcium carbonate (0.324 g, 3.2 mmol). The reaction, being deemed complete after 30 min by TLC analysis, was quenched by addition hydrochloric acid (1 N, 30 mL). After the methanol and dichloromethane were removed under vacuum, the remaining was extracted with chloroform using a phase separator. The organic solvent was removed in vacuo to yield methyl 4-(3,5-dibromo-4-hydroxyphenyl)butanoate (520 g, 88%).

### Description 4

### Methyl (3,5-dibromo-4-hydroxy-benzoylamino) acetate

3,5-Dibromo-4-hydroxybenzoic acid (5.1 g, 17.23 mmol) was refluxed in thionyl chloride (100 mL) for 6 h. The reaction mixture was cooled and the excess thionyl chloride removed. The product was used in the next step without further purification.

Glycine methyl ester hydrochloride (4.33 g, 34.5 mmol) was dissolved in dichloromethane (430 mL) and triethyl amine (20 mL, 143.6 mmol). The acid chloride (17.23 mmol) was added in small portions. Stirring was continued overnight. The solvent was evaporated. The reaction mixture was dissolved in dichloromethane and washed with hydrochloric acid (0.1 M, aqueous solution). The organic phase was dried over sodium sulphate, filtered and the solvent removed. A small amount of ethyl acetate was added and the mixture was filtered to give 4.21 g (88%) of almost pure compound. The product was crystallized from heptanes/ethyl acetate to give 2.5 g of the title compound (52% yield) as a white powder.

### Description 5

### Methyl 2-fluoro-3-(4-hydroxy-3,5-dibromophenyl) propanoate

To a mixture of 2-hydroxy-3-(4-hydroxy-phenyl)-propanoic acid (4 g, 22 mmol) in dry methanol (150 mL) was added hydrochloric acid (2 mL). The reaction mixture was heated at reflux for 3 hours, cooled to room temperature and concentrated under vacuum. The residue was dissolved in ethyl acetate (200 mL) and washed with sodium hydrogen carbonate aqueous solution (saturated) and brine, then dried over magnesium sulphate. The pure compound methyl 2-hydroxy-3-(4-hydroxy-phenyl)-propanoate (4.10 g) was obtained in 95% of yield.

To a solution of methyl 2-hydroxy-3-(4-hydroxy-phenyl)-propanoate (2.1 g, 10.7 mmol) in acetonitrile (130 mL) was added bromine (3.4 g, 21.4 mmol) in acetonitrile (20 mL) drop wise at room temperature, and the reaction mixture was stirred overnight. After evaporation of the solvent, the residue was dissolved in ethyl acetate (150 mL) and washed with washed with NaHSO₃ (aq.) and brine dried over magnesium sulphate and concentrated in vacuo to give methyl 2-hydroxy-3-(4-hydroxy-3,5-dibromophenyl) propanoate (3.14 g, 83%).

The mixture of methyl 2-hydroxy-3-(4-hydroxy-3,5-dibromophenyl) propanoate (3.4 g, 9.5 mmol) and potassium carbonate (1.45 g, 1051 mmol) in acetone (85 mL) were added to methyl iodide (1.5 g, 10.5 mmol). The mixture was stirred at 60° C. overnight. After cooling to room temperature, acetone was removed and hydrochloric acid aqueous solution (1 M) was added and extracted with ethyl acetate (3×150 mL). The organic layer was washed with sodium hydrogen carbonate aqueous solution (saturated) and brine and then dried over magnesium sulphate. The reaction mixture was evaporated to get crude methyl 2-hydroxy-3-(4-methoxy-3,5-dibromophenyl) propanoate and used directly in next step without additional purification.

A solution of crude methyl 2-hydroxy-3-(4-methoxy-3,5-dibromophenyl) propanoate (3.7 g, 10.2 mmol) in dry dichloromethane (30 mL) was added slowly to the solution of DAST (Et₂NSF₃) (1.76 g, 10.9 mmol) in dry dichloromethane (10 mL) at 0° C. under nitrogen atmosphere. The mixture was stirred for 15 min and allowed to come to room temperature and poured into a mixture of water and ice. The organic layer was separated and the water was extracted with dichloromethane (2×30 mL). The combined organic layers were washed with water and dried over magnesium sulphate. The obtained residue was purified by flash chromatography (ethyl acetate/heptane 5:95). Methyl 2-fluoro-3-(4-methoxy-3,5-dibromophenyl) propanoate (2.4 g) was obtained in 65% yield.

To a dichloromethane (30 mL) solution of methyl 2-fluoro-3-(4-methoxy-3,5-dibromophenyl) propanoate (2.4 g, 6.5 mmol) at −78° C. was added BF₃.SMe₂ (40 mL) very slowly. The mixture was allowed to warm up to room temperature and stirred overnight. The reaction mixture was diluted with brine and extracted with ethyl acetate (3×20 mL). The combined organic phases were dried (magnesium sulphate), filtered and concentrated. The obtained residue was purified by flash chromatography to give the title compound in 70% yield (1.6 g).

### Description 6

### Ethyl (E)-3-(3,5-dibromo-4-hydroxy-phenyl)-acrylate

2,6-Dibromo-4-nitro-phenol (5.0 g) was dissolved in ethyl acetate (100 ml). To this solution, platinium oxide (0.3 g) was added and hydrogen was passed through the reaction mixture overnight while stirring. The catalyst was removed by filtration through celite and washed with ethyl acetate (50 ml). The filtrate was concentrated under reduced pressure and diluted with 200 ml of diethyl ether. Hydrogen chloride gas was passed through the solution and the precipitates were collected to afford 4.8 g of 2,6-dibromo-4-amino-phenol hydrochloride as pale-yellow salts.

A stirred suspension of 2,6-dibromo-4-amino-phenol hydrochloride (4.8 g, 16 mmol) in hydrochloric acid (37%, 20 ml) was cooled to −5° C. in an ice/sodium chloride bath. To this suspension, a solution of NaNO₂ (1.14 g) in water (3 ml) was added carefully. The resultant mixture was stirred for 20 min at this temperature. Then a solution of potassium iodide (2.89 g) in water (3 ml) was added drop wise. After the addition, the ice-bath was removed and the reaction mixture was stirred for 20 min at room temperature and then 20 min at 80° C. Then the mixture was cooled down and extracted with ethyl acetate (3×20 mL). The extracts were combined, washed consecutively with brine and sodium hydrogen carbonate aqueous solution (saturated aqueous solution), dried over anhydrous magnesium sulphate, and concentrated. The residue was purified by using grading column chromatography (ethyl acetate/heptanes 2:98 to 5:95) to give 2,6-dibromo-4-iodo-phenol (3.2 g and 52% yield).

Palladium acetate (100 mg), triphenyl phosphine (350 mg) and acetonitrile (9 ml) were added to a nitrogen charged flask. The mixture was stirred at room temperature under nitrogen for 10 min until a yellow suspension was formed. Then, 2,6-dibromo-4-amino-phenol hydrochloride (3.2 g), ethyl acrylate (1.1 ml), triethyl amine (2.37 ml) and additional acetonitrile (9 ml) were added to the mixture. The resulting dark red mixture was placed in an oil bath and stirred overnight at 60° C. under nitrogen. After that the mixture was allowed to cool to room temperature, and then filtered through celite. The celite plug was rinsed with ethyl acetate (100 ml). The filtrates and all the rinses were combined and washed with hydrochloric acid (1 M, aqueous solution), brine, saturated sodium hydrogen carbonate aqueous solution, and brine in sequence, dried over magnesium sulphate and concentrated. The residue was purified by column chromatography (ethyl acetate/heptanes 5:95 to 7:93) and further purified by recrystallization with diethyl ether/hexane to give the analytical pure product of ethyl (E)-3-(3,5-dibromo-4-hydroxy-phenyl)-acrylate.

### Description 7

### 2,6-Dibromo-4-amino-phenol

A mixture of 2,6-dibromo-4-nitro-phenol (10 g, 33.4 mmol) and tin(II) chloride dihydrate (15.5 g, 66.8 mmol, 5 eq.) in methanol (200 mL) was heated to 70° C. for 7 h. The reaction mixture was evaporated to remove the solvent, the residue was dissolved in ethyl acetate (200 ml), sodium hydrogen carbonate aqueous solution (saturated) was dropped in and the formed precipitate was removed by filtration. The organic layer was washed with brine (3×50 ml), dried over magnesium sulphate and the solvent evaporated by reduced pressure. The residue was purified with flash chromatography, (heptane/ethyl acetate/triethylamine 60:40:0.1), to give the wanted 4-amino-2,6-dibromo-phenol (8.40 g, yield: 93%).

### Description 8

### Methyl N-(3,5-Dibromo-4-hydroxy-phenyl)-oxalate

To a solution of 4-amino-2,6-dibromo-phenol (Description 7, 1068 mg, 4 mmol) in dry dichloromethane (50 mL), triethylamine (404 mg, 4 mmol), and methyl oxalylchloride (490 mg, 4 mmol) were added. The mixture was stirred at 0° C. for 3 h. The reaction was quenched with water. The mixture was extracted with ethyl acetate (3×50 mL). The combined organic phases were dried with magnesium sulphate, filtered and the solvent removed under vacuum. The residue was purified by flash chromatography (n-heptane/ethyl acetate 60:40) to afford the title compound methyl N-(3,5-dibromo-4-hydroxy-phenyl)-oxalate in 39% yield (550 mg) (MW=353). LC/MS (ESI): m/z 354.2 (M+1).

### Description 9

### Methyl N-(3,5-Dibromo-4-hydroxy-phenyl)-malonate

To a solution of 4-amino-2,6-dibromo-phenol (Description 7, 2.13 g, 8 mmol) in dry dichloromethane (70 mL), triethylamine (808 mg, 8 mmol), and methyl malonylchloride (1092 mg, 8 mmol) were added. The mixture was stirred at 0° C. for 3 h. The reaction was quenched with water. The mixture was extracted with ethyl acetate (3×100 mL). The combined organic phases were dried with magnesium sulphate, filtered and the solvent removed under vacuum. The residue was purified by flash chromatography (n-heptane/ethyl acetate 60:40) to afford the title compound methyl N-(3,5-dibromo-4-hydroxy-phenyl)-malonate in 64% yield (1879 mg) (MW=367). LC/MS (ESI): m/z 368.0 (M+1), 365.8 (M−1).

### Description 10

### 5-Trifluoromethyl-3-nitrobenzylbromide

5-Trifluoromethyl-3-nitrobenzoic acid (0.7 g, 3.0 mmol) was dissolved in methanol and 10 drops of sulphuric acid (conc.) were added, and the reaction was stirred over night at reflux temperature. Methanol was removed and the residue re-dissolved in dichloromethane and washed with water. The solvent was dried (magnesium sulphate) and removed under vacuum to give 0.71 g of 5-trifluoromethyl-3-nitrobenzoate methyl ester.

To lithium aluminium hydride (0.32 g, 8.7 mmol) in tetrahydrofuran (8 mL) was carefully, and drop wise, added a solution of 5-trifluoromethyl-3-nitrobenzoate methyl ester in tetrahydrofuran (2 mL) and stirred at room temperature over night. The reaction was quenched with careful addition of water (20 mL) then acidified using hydrochloric acid (3 M) and finally extracted with diethylether (3×50 mL). The combined organic phases were dried (magnesium sulphate) and the solvent was removed under vacuum. The residue was purified on silica gel column (diethylether/heptane 1:3) to provide 0.35 g, (55%) of 5-trifluoromethyl-3-nitrobenzylalcohol.

5-Trifluoromethyl-3-nitrobenzylalcohol was dissolved in 3 mL toluene and 0.1 mL PBr₃ was added with a syringe and the reaction was stirred at room temperature over night. The reaction was filtered through a plug of silica which was washed with diethylether. The solvent was removed under vacuum to give 0.38 g (85% yield) of the title compound.

### Description 11

### 5-Methyl-3-nitrobenzylbromide

5-Methyl-3-nitrotoluene (0.5 g, 3.3 mmol) and NBS (0.6 g, 3.3 mmol) were dissolved in CCl₄ and benzoylperoxide (10 mg) was added. The reaction was refluxed over night and then cooled to room temperature. The reaction mixture was filtered and the solvent evaporated after which the residue was dissolved in dichloromethane and filtered through a plug of silica. The obtained residue was a 2:1 mixture of the corresponding 5-methyl-3-nitrobenzylbromide and starting material. The yield was calculated to 65%.

### Description 12

### 5-Chloro-3-nitrobenzylbromide

5-Chloro-3-nitrotoluene (synthesized following Journal of Medicinal Chemistry, 2000, 43, 4733) (0.33 g, 1.9 mmol) and NBS (0.34 g, 1.9 mmol) were dissolved in 9 mL of CCl₄ and 10 mg of benzoylperoxide were added. The reaction was refluxed over night and then cooled to room temperature. The reaction mixture was filtered and the solvent evaporated after which the residue was dissolved in dichloromethane and was filtered through a plug of silica. The solvent was again evaporated to give 0.55 g crude product containing starting material the monobrominated and the dibrominated benzyl compound. Purification on silica (diethylether/heptane 9:1) gave 0.13 g (27% yield) of 5-chloro-3-nitrobenzylbromide.

### Description 13

### Methyl (S)-2-(2-(3,5-dichloro-4-{[3-(ethylamino)-5-methylbenzyl]oxy}phenyl)acetylamino)-2-phenyl-acetate

A solution of (3,5-dichloro-4-{[3-(ethylamino)-5-methyl-benzyl]oxy}phenyl)acetic acid (20 mg, 0.054 mmol), 3-ethyl-1-[3-(dimethylamino)propyl]carbodiimide hydrochloride (EDCI), (21 mg, 0.108 mmol), 1-hydroxybenzotriazole hydrate (HOBT), (17 mg, 0.108 mmol), (S)-(+)-2-phenylglycine methyl ester hydrochloride (22 mg, 0.108 mmol) and triethylamine (17 mg, 0.108 mmol) in dimethylformamide (2 ml) was stirred at room temperature for 24 hrs. The resulting reaction mixture was diluted with brine and extracted with ethyl acetate (3×15 mL). The combined organic phases were dried with magnesium sulphate, filtered and the solvent removed under vacuum. The residue was purified by flash chromatography (n-heptane/ethyl acetate 1:1) to afford methyl (S)-2-{2-(3,5-dichloro-4-{[3-(ethylamino)-5-methylbenzyl]oxy}phenyl)acetylamino}-2-phenyl-acetate in 52% yield (14 mg) (MW=515.4). LC/MS (ESI): m/z 515.6 (M).

### Description 14

### Methyl 3-[3,5-dichloro-4-(3-iodobenzyloxy)phenyl]propanoate

To a mixture of potassium carbonate (0.70 g, 5.0 mmol) and methyl 3-(4-hydroxy-3,5-dichlorophenyl) propanoate (0.25 g, 1.0 mmol) dissolved in 30 mL of acetone was added 3-iodobenzyl bromide (0.3 g, 1.0 mmol). The reaction was refluxed overnight. The combined organic phases were dried (magnesium sulphate) and the solvent was removed under vacuum. The residue was purified on silica gel column (diethylether/heptane 1:3) to provide 0.37 g (80% yield) of methyl 3-[3,5-dichloro-4-(3-iodobenzyloxy)phenyl]propanoate.

### Description 15

### 1,3-Dibromo-5-methyl-2-[(E)-2-(3-nitro-phenyl)-vinyl]-benzene

To 2,6-dibromo-4-methyl-benzaldehyde (prepared from literature procedure JOC, 2003, 5384) (0.31 g, 1.28 mmol) in DMPU (13 mL) was added sodium hydride (0.083 g, 2.06 mmol) the mixture was stirred for 5 min. The (3-nitro-benzyl)-phosphonic acid dimethyl ester (0.47 g, 1.29 mmol), (prepared from literature procedure JMC, 2004, 2095) was added at 0° C. and the reaction was stirred for 2 hours. Water and ethyl acetate was added, the organic phase collected and dried. The solvents were distilled off and the product purified on silica (ethyl ether/heptane 1:3) to give 0.45 g (88% yield) of 1,3-dibromo-5-methyl-2-[(E)-2-(3-nitro-phenyl)-vinyl]-benzene.

### Description 16

### {3,5-Dibromo-4-[(E)-2-(3-nitro-phenyl)-vinyl]-phenyl}-methanol

To 1,3-dibromo-5-methyl-2-[(E)-2-(3-nitro-phenyl)-vinyl]-benzene (Description 15, 0.070 g, 0.17 mmol) in CCl₄, 1 mL was added NBS, (0.030 g, 0.17 mmol) the mixture was stirred at reflux for 15 h. Filtration throw silica with dichloromethane evaporation of solvents gave a crude product which was dissolved in dioxane (3 mL) and potassium hydroxide (6 mL, aq., 2M) and refluxed overnight. Ethyl acetate was added to extract the product, which in turn was dried and evaporated to give a (3:1) mixture of starting material and (3,5-dibromo-4-[(E)-2-(3-nitro-phenyl)-vinyl]-phenyl)-methanol. The product was purified on silica (diethyl ether/heptane, 1:1) to give 0.016 g (23% yield) of {3,5-dibromo-4-[(E)-2-(3-nitro-phenyl)-vinyl]-phenyl)}-methanol.

### Description 17

### {3,5-Dibromo-4-[(E)-2-(3-nitro-phenyl)-vinyl]-benzyloxy}-acetic acid tert-butyl ester

To {3,5-dibromo-4-[(E)-2-(3-nitro-phenyl)-vinyl]-phenyl}-methanol (Description 16, 0.016 g, 0.04 mmol) in tetrahydrofuran (1 mL) was added sodium hydride (0.003 g, 0.08 mmol). The mixture was stirred 5 min, tert-butyl bromoacetate was added and the reaction was stirred for 15 h. Ethyl acetate and water were added and the product was extracted, dried and evaporated. The residue was purified on silica (diethyl esther/heptane, 1:3) to give 0.010 g (60% yield) of {3,5-dibromo-4-[(E)-2-(3-nitro-phenyl)-vinyl]-benzyloxy}-acetic acid tert-butyl ester.

### General Procedure for the Preparation of Examples 1-11

A mixture of the appropriate phenol (e.g. methyl 3-(3,5-dihalo-4-hydroxyphenyl) propionate) (I eq.), the appropriate 3-nitrobenzylbromide (1 eq.) and potassium carbonate (5 eq.) in dry acetone (30 mL/mmol phenol) was heated to 56° C. and stirred for 20 h. The reaction mixture was concentrated, diluted with ethyl acetate and washed with water. The organic phase was dried, evaporated and purified on a column (silica, 100% dichloromethane) to give the nitro derivative (e.g. methyl 3-[3,5-dihalo-4-(3-nitrobenzyloxy)phenyl]propionate).

A mixture of the nitro derivative (e.g. methyl 3-[3,5-dihalo-4-(3-nitrobenzyloxy)phenyl]propionate) and tin(II) chloride dihydrate (5 eq.) in absolute ethanol (40 mL/mmol ester) was heated to 75° C. for 4 h. The reaction mixture was quenched with sodium hydrogen carbonate aqueous solution (saturated). The aqueous phase was extracted with ethyl acetate (3×40 mL) and the combined organic phases were washed with water and brine and dried over magnesium sulphate. After evaporation of the solvent, the residue was purified by flash chromatography (dichloromethane/diethylether 90:10) to yield the wanted amino derivative (e.g. methyl 3-[3,5-dihalo-4-(3-aminobenzyloxy)phenyl]propionate).

The amino derivative (e.g. methyl 3-[3,5-dihalo-4-(3-aminobenzyloxy)phenyl]propionate) was dissolved in dioxane (5 mL), potassium hydroxide (2 M in water, 5 eq.) (sodium hydroxide and lithium hydroxide have also been used) was added and the mixture was stirred at room temperature over night. After acidification with hydrochloric acid (1 M) the product was extracted into ethyl acetate. The solvent was evaporated under vacuum to yield the expected acid (e.g. 3-[3,5-dihalo-4-(3-aminobenzyloxy)phenyl]propionic acid).

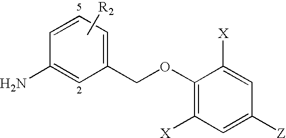

| | | | | Yield | MW | M + 1 |
|---|---|---|---|---|---|---|
| Example | Z | R₂ | X | (%) | (calc) | (found)* |
| 1 | 3-Carboxypropyl | H | Cl | 27 | 340.2 | 340.2 |
| 2 | 3-Carboxypropyl | 5-CF₃ | Br | 99 | 497.1 | 496.4 |
| | | | | | | (M − 1) |
| 3 | 3-Carboxypropyl | 5-Cl | Br | 40 | 463.2 | 464.0 |
| 4 | 3-Carboxypropyl | 5-Me | Br | 29 | 443.1 | 444.5 |
| 5 | 3-Carboxypropyl | 4-Me | Br | 42 | 443.1 | 444.5 |
| 6 | Carboxymethyl | H | Br | 14 | 401.0 | 402.2 |
| 7 | N-(Carboxymethyl)formamido | H | Br | 13 | 458.1 | 457.1 |
| | | | | | | (M − 1) |
| 8 | 2-Carboxyethyl | 5-Me | Cl | 58 | 340.2 | 340.0 |
| | | | | | | (M) |
| 9 | N-(Carboxymethyl)formamido | 5-Me | Cl | 67 | 383.2 | 383.3 |
| | | | | | | (M) |
| 10 | 3-Carboxypropyl | 2-Cl | Br | 6 | 463.5 | 462.2 |
| | | | | | | (M − 1) |
| 11 | 3-Carboxypropyl | 2-F | Br | 10 | 447.1 | 446.0 |
| | | | | | | (M − 1) |

*Analyzed on HPLC-MS with alternating +/− API and equiped with different brands of 50 mm *2.1 mm, 5μ C8 columns. Eluted with 0.05% formic acid/ACN or 0.05% ammonium acetate/ACN
*MW calc. (molecular weight) is an isotopic average and the “found mass” is referring to the most abundant isotope detected in the LC-MS. The “found mass” refers to M + 1 unless specified otherwise.

### General Procedure for the Preparation of Examples 12-121

### Method A

Sodium cyanoborohydride (1.1 eq.) was added to the solution of the appropriate aniline (e.g. methyl 3-[3,5-dihalo-4-(3-aminobenzyloxy)phenyl]propionate) (1 mmol) and the appropriate aldehyde (or ketone) (1.1 eq. 1) in methanol (25 mL/mmol aniline). The mixture was stirred at room temperature overnight. The reaction mixture was poured into water (50 mL) and extracted with dichloromethane (3×25 mL). The organic phases were combined and dried (magnesium sulphate), the solvent was removed under vacuum and the residue was purified by flash chromatography (100% dichloromethane) to provide the desired secondary amine (e.g. methyl 3-[3,5-dihalo-4-(3-alkylaminobenzyloxy)phenyl]propionate).

The amine (e.g. methyl 3-[3,5-dihalo-4-(3-alkylaminobenzyloxy)phenyl]propionate) was dissolved in dioxane (7 mL/mmol of ester), sodium hydroxide (lithium hydroxide has also been used) (1 M in water, 10 eq.) was added and the mixture was stirred at room temperature over night. After acidification with hydrochloric acid (1 M) the product was extracted with ethyl acetate. The solvent was evaporated under vacuum to yield the acid (e.g. 3-[3,5-dihalo-4-(3-alkylaminobenzyloxy)phenyl]propionic acid).

### Method B

Sodium cyanoborohydride (0.3 mmol) was added to the solution of the appropriate aniline (e.g. methyl 3-[3,5-dihalo-4-(3-aminobenzyloxy)phenyl]propionate) (0.1 mmol) and the appropriate aldehyde (or ketone) (0.12-1.0 mmol) in methanol:tetrahydrofuran 2:1 (1.5 mL). The mixture was stirred at room temperature from 24 h-96 h. The reaction mixture was purified by semi-preparative-HPLC (Zorbax CombiHT (SB-C8, 50μ×21.2 mm) Mobile Phase: Solvent A: Water with 0.5% formic acid; Solvent B: acetonitrile. Gradient: 2 min 80% of A then over 8 min to 5% of A) to yield the desired secondary amine (e.g. methyl 3-[3,5-dihalo-4-(3-alkylaminobenzyloxy)phenyl]propionate).

The amine (e.g. methyl 3-(3,5-dihalo-4-(3-alkylaminobenzyloxy)phenyl) propionate) was dissolved in THF (0.25 mL), Lithium hydroxide (1 M in water, 0.25 mL) was added and the mixture was stirred at room temperature over night. The mixture was evaporated to dryness, re-dissolved in water (1 ml), and applied on a C-18 SPE-column (Isolute 0.5 g). Salts were washed away with water (10 ml) and the product was eluted with 60% aq. methanol (5 ml) and the solvent was evaporated under vacuum to yield the acid (e.g. 3-[3,5-dihalo-4-(3-alkylaminobenzyloxy)phenyl]propionic acid). Products were obtained as their Lithium salts.

### Method C

The amine (e.g. methyl (E)-3-{4-[(2,5-dichloro-3-amino-benzyl)oxy]-3,5-dibromophenyl}acrylate) (0.37 mmol) was dissolved in dry tetrahydrofurane (5 ml) and dry dichloromethane (5 ml) in a vial. Dry acetone (1 ml) and acetic acid (50 μl) were added and the mixture stirred for 1 minute. The solution was cooled to 0° C. and BH₃.SMe₂ (925 μl, 2M) was added portionwise. The vial was sealed and stirred at room temperature overnight. The solvents were evaporated and the crude was dissolved in tetrahydrofurane (5 ml) and treated with lithium hydroxide (4 ml, 1 M) and stirred overnight. The mixture was evaporated to dryness, re-dissolved in tetrahydrofuran/water (1 ml) and, if needed, fitered through a C-18 SPE-column (Isolute 0.5 g). The residue was purified by semi-preparative-HPLC (Zorbax CombiHT (SB-C8) Mobil Phase: Solvent A. Water with 0.5% formic acid; μ50×21.2 mm, 5 Solvent B: acetonitrile. Gradient: 2 min 80% of A then over 8 min to 5% of A) to yield the desired amine (e.g. (E)-3-(3,5-dibromo-4-{[2,5-dichloro-3-(ethylamino)benzyl]oxy}phenyl)acrylic acid).

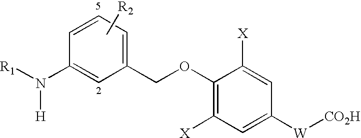

| | | | | | Yield | MW | M + 1 | |
|---|---|---|---|---|---|---|---|---|
| Example | R₁ | R₂ | X | W | (%) | (calc) | (found)* | Method |
| 12 | Me | H | Br | (CH₂)₂ | 31 | 443.1 | 444 | B |
| 13 | Me | 5-CF₃ | Br | (CH₂)₂ | 50 | 511.1 | 512.0 | B |
| 14 | Me | 2-Me | Br | (CH₂)₂ | 52.6 | 457.2 | 458.0 | B |
| 15 | Et | 4-Me | Br | (CH₂)₂ | 13 | 471.2 | 470.6 | A |
| | | | | | | | (M − 1) | |
| 16 | Et | 2-Me | Br | (CH₂)₂ | 17 | 471.2 | 470.6 | A |
| | | | | | | | (M − 1) | |
| 17 | Et | 5-CF₃ | Br | (CH₂)₂ | 59 | 525.2 | 526.4 | A |
| 18 | Et | 5-Cl | Br | (CH₂)₂ | 93 | 491.6 | 492.5 | A |
| 19 | Et | 5-Me | Br | (CH₂)₂ | 54 | 471.2 | 472.1 | A |
| 20 | n-Pr | H | Br | (CH₂)₂ | 17 | 471.2 | 472.0 | B |
| 21 | n-Pr | 2-Me | Br | (CH₂)₂ | 58 | 485.2 | 486.0 | B |
| 22 | 2-Butenyl | H | Br | (CH₂)₂ | 13 | 483.2 | 484.0 | B |
| 23 | i-Pr | H | Br | (CH₂)₂ | 42 | 471.2 | 472 | B |
| 24 | i-Pr | 5-CF₃ | Br | (CH₂)₂ | 64 | 539.2 | 540.0 | B |
| 25 | i-Bu | H | Br | (CH₂)₂ | 21 | 485.2 | 486.0 | B |
| 26 | n-Bu | H | Br | (CH₂)₂ | 17 | 485.2 | 486.0 | B |
| 27 | 2-Methylthioethyl | H | Br | (CH₂)₂ | 16 | 517.3 | 518.0 | B |
| 28 | 2-t-Bu-ethyl | H | Br | (CH₂)₂ | 48 | 513.3 | 514.0 | B |
| 29 | sec-Bu | H | Br | (CH₂)₂ | 77.5 | 485.2 | 486.0 | B |
| 30 | sec-Bu | 2-Me | Br | (CH₂)₂ | 92 | 499.2 | 500.0 | B |
| 31 | Cyclobutyl | H | Br | (CH₂)₂ | 91.4 | 483.2 | 484.0 | B |
| 32 | Cyclobutyl | 2-Me | Br | (CH₂)₂ | 35 | 497.2 | 498 | B |
| 33 | Cyclopropylmethyl | H | Br | (CH₂)₂ | 44 | 483.2 | 484.0 | B |
| 34 | Cyclohexylmethyl | H | Br | (CH₂)₂ | 33 | 525.3 | 526 | B |
| 35 | 2-Ethyl-butyl | H | Br | (CH₂)₂ | 9 | 513.0 | 512.0 | B |
| | | | | | | | (M − 1) | |
| 36 | Me | H | Cl | (CH₂)₂ | 19 | 354.2 | 354.0 | B |
| | | | | | | | (M) | |
| 37 | Et | H | Cl | (CH₂)₂ | 26 | 368.2 | 368.0 | B |
| | | | | | | | (M) | |
| 38 | Et | 5-CF₃ | Cl | (CH₂)₂ | 63 | 436.2 | 437 | A |
| 39 | n-Pr | H | Cl | (CH₂)₂ | 54.8 | 382.2 | 382.0 | B |
| | | | | | | | (M) | |
| 40 | i-Pr | H | Cl | (CH₂)₂ | 76 | 382.2 | 382.0 | B |
| | | | | | | | (M) | |
| 41 | sec-Bu | H | Cl | (CH₂)₂ | 82 | 396.3 | 396.0 | B |
| | | | | | | | (M) | |
| 42 | cyclobutyl | H | Cl | (CH₂)₂ | 70 | 394.2 | 394.0 | B |
| | | | | | | | (M) | |
| 43 | Cyclopropylmethyl | H | Cl | (CH₂)₂ | 49 | 394.3 | 394.0 | B |
| | | | | | | | (M) | |
| 44 | Et | H | Br | CONH—CH₂ | 63.6 | 486.1 | 487.0 | B |
| 45 | Et | H | Cl | CONH—CH₂ | 24 | 398.2 | 399.2 | A |
| 46 | Me | H | Br | CONH—CH₂ | 50.2 | 472.1 | 473.0 | B |
| 47 | n-Pr | H | Br | CONH—CH₂ | 84 | 500.2 | 501.0 | B |
| 48 | i-Pr | H | Br | CONH—CH₂ | 90.2 | 500.2 | 501.0 | B |
| 49 | Cyclobutyl | H | Br | CONH—CH₂ | 60.4 | 512.2 | 513.0 | B |
| 50 | sec-Bu | H | Br | CONH—CH₂ | 88.6 | 514.2 | 515.0 | B |
| 51 | Et | 5-Me | Cl | CONH—CH₂ | 61 | 411.2 | 413.0 | A |
| | | | | | | | (M + 2) | |
| 52 | Et | H | Br | CH₂ | 80 | 443.1 | 444.1 | A |
| 53 | Et | 5-Cl | Br | CH₂ | 47 | 477.7 | 478.1 | A |
| | | | | | | | (M) | |
| 54 | Et | 5-Me | Cl | CH₂ | 59 | 368.2 | 368.3 | A |
| | | | | | | | (M) | |
| 55 | Et | 5-CFH₂ | Br | (CH₂)₂ | 14 | 489.1 | 488.3 | B |
| | | | | | | | (M − 1) | |
| 56 | Et | 5-CH₂-OEt | Br | (CH₂)₂ | 18 | 515.2 | 514.4 | B |
| | | | | | | | (M − 1) | |
| 57 | Et | 2-F | Br | (CH₂)₂ | 10 | 447.1 | 446.0 | B |
| | | | | | | | (M − 1) | |
| 58 | Et | 2-Ome | Br | (CH₂)₂ | 5 | 487.1 | 486.0 | B |
| | | | | | | | (M − 1) | |
| 59 | Et | 2-OEt | Br | (CH₂)₂ | 3 | 501.2 | 500.0 | B |
| | | | | | | | (M − 1) | |
| 60 | (CH₂)₂—CO₂H | H | Cl | (CH₂)₂ | 54 | 412.2 | 410.3 | B |
| | | | | | | | (M − 2) | |
| 61 | Me | 5-Cl | Br | (CH₂)₂ | 11 | 477.6 | 478.0 | B |
| | | | | | | | (M) | |
| 62 | Me | 5-Me | Br | (CH₂)₂ | 20 | 457.2 | 458.0 | B |
| 63 | i-Pr | 5-Cl | Br | (CH₂)₂ | 33 | 505.6 | 506.0 | B |
| | | | | | | | (M) | |
| 64 | i-Pr | 5-Me | Br | (CH₂)₂ | 45 | 485.2 | 486.0 | B |
| 65 | Cyclobutyl | 5-CF₃ | Br | (CH₂)₂ | 43 | 551.2 | 552.0 | B |
| 66 | Cyclobutyl | 5-Cl | Br | (CH₂)₂ | 83 | 517.7 | 518.0 | B |
| | | | | | | | (M) | |
| 67 | Cyclobutyl | 5-Me | Br | (CH₂)₂ | 17 | 497.2 | 498.0 | B |
| 68 | sec-Bu | 5-CF₃ | Br | (CH₂)₂ | 24 | 553.2 | 554.0 | B |
| 69 | sec-Bu | 5-Cl | Br | (CH₂)₂ | 28 | 519.7 | 520.0 | B |
| | | | | | | | (M) | |
| 70 | sec-Bu | 5-Me | Br | (CH₂)₂ | 50 | 499.2 | 500.0 | B |
| 71 | i-Pr | 5-Me | Cl | (CH₂)₂ | 62 | 396.3 | 396.0 | B |
| | | | | | | | (M) | |
| 72 | i-Pr | 2-Cl | Br | (CH₂)₂ | 30 | 505.6 | 506.0 | B |
| | | | | | | | (M) | |
| 73 | i-Pr | 6-F | Br | (CH₂)₂ | 12 | 489.2 | 490.0 | B |
| 74 | Cyclobutyl | 5-Me | Cl | (CH₂)₂ | 67 | 408.2 | 408.0 | B |
| | | | | | | | (M) | |
| 75 | Cyclobutyl | 2-Cl | Br | (CH₂)₂ | 8 | 517.7 | 518.0 | B |
| | | | | | | | (M) | |
| 76 | Cyclobutyl | 6-F | Br | (CH₂)₂ | 22 | 501.2 | 502.0 | B |
| 77 | sec-Bu | 5-Me | Cl | (CH₂)₂ | 8 | 410.2 | 410.0 | B |
| | | | | | | | (M) | |
| 78 | sec-Bu | 2-Cl | Br | (CH₂)₂ | 9 | 519.7 | 520.0 | B |
| | | | | | | | (M) | |
| 79 | sec-Bu | 6-F | Br | (CH₂)₂ | 41 | 503.2 | 504.0 | B |
| 80 | Et | 6-F | Br | (CH₂)₂ | 10 | 475.2 | 476.0 | B |
| 81 | Et | 2,5-Cl | Br | (CH₂)₂ | 18 | 526.1 | 526.0 | B |
| | | | | | | | (M) | |
| 82 | i-Pr | 2,5-Cl | Br | (CH₂)₂ | 57 | 540.1 | 540.0 | B |
| | | | | | | | (M) | |
| 83 | Cyclobutyl | 2,5-Cl | Br | (CH₂)₂ | 47 | 552.1 | 552.0 | B |
| | | | | | | | (M) | |
| 84 | 1,2-Dimethylpropyl | 5-Cl | Br | (CH₂)₂ | 14 | 533.7 | 534.0 | B |
| | | | | | | | (M) | |
| 85 | 1,2-Dimethylpropyl | 2-Cl | Br | (CH₂)₂ | 12 | 533.6 | 534.0 | B |
| | | | | | | | (M) | |
| 86 | Et | 5-Me | Cl | (CH₂)₂ | 21 | 382.1 | 382.0 | B |
| | | | | | | | (M) | |
| 87 | Et | 5-CN | Br | (CH₂)₂ | 10 | 482.2 | 483.0 | B |
| 88 | Cyclobutyl | 5-CN | Br | (CH₂)₂ | 3 | 508.2 | 509.2 | B |
| 89 | Et | 2-Cl | Br | (CH₂)₂ | 10 | 491.7 | 492.2 | B |
| | | | | | | | (M) | |
| 90 | i-Pr | 5-CF₃ | Br | CONH—CH₂ | 6 | 568.2 | 569.0 | B |
| 91 | i-Pr | 5-Me | Br | CONH—CH₂ | 34 | 514.2 | 515.1 | B |
| 92 | i-Pr | 5-Cl | Br | CONH—CH₂ | 20 | 534.6 | 535.0 | B |
| | | | | | | | (M) | |
| 93 | cyclobutyl | 5-CF₃ | Br | CONH—CH₂ | 17 | 580.2 | 581.0 | B |
| 94 | cyclobutyl | 5-Me | Br | CONH—CH₂ | 14 | 526.2 | 527.1 | B |
| 95 | cyclobutyl | 5-Cl | Br | CONH—CH₂ | 9 | 546.6 | 545.2 | B |
| | | | | | | | (M − 1) | |
| 96 | Et | 5-Cl | Br | CONH—CH₂ | 15 | 520.7 | 521.0 | B |
| | | | | | | | (M) | |
| 97 | sec-Bu | 5-CF₃ | Br | CONH—CH₂ | 26 | 582.2 | 583.0 | B |
| 98 | sec-Bu | 5-Me | Br | CONH—CH₂ | 26 | 528.3 | 529.1 | B |
| 99 | sec-Bu | 5-Cl | Br | CONH—CH₂ | 5 | 548.7 | 549.0 | B |
| | | | | | | | (M) | |
| 100 | Et | 5-Me | Br | CONH—CH₂ | 16 | 500.2 | 501.0 | B |
| 101 | Et | 5-CF₃ | Br | CONH—CH₂ | 6 | 554.2 | 555.4 | B |
| 102 | 1,2-Dimethylpropyl | 5-Cl | Br | CONH—CH₂ | 3 | 562.7 | 563.1 | B |
| | | | | | | | (M) | |
| 103 | Et | 2,5-Cl | Br | CONH—CH₂ | 3 | 555.1 | 555.0 | B |
| | | | | | | | (M) | |
| 104 | i-Pr | 2,5-Cl | Br | CONH—CH₂ | 3 | 569.1 | 569.0 | B |
| | | | | | | | (M) | |
| 105 | cyclobutyl | 2,5-Cl | Br | CONH—CH₂ | 2 | 581.1 | 583.0 | B |
| | | | | | | | (M + 2) | |
| 106 | Et | 2-Cl | Br | CONH—CH₂ | 15 | 520.8 | 521.0 | B |
| | | | | | | | (M) | |
| 107 | i-Pr | 2-Cl | Br | CONH—CH₂ | 12 | 534.9 | 535.0 | B |
| | | | | | | | (M) | |
| 108 | cyclobutyl | 2-Cl | Br | CONH—CH₂ | 10 | 546.6 | 547.0 | B |
| | | | | | | | (M) | |
| 109 | Et | 5-Cl | Br | CH₂—CHF | 43 | 509.6 | 510.1 | B |
| | | | | | | | (M) | |
| 110 | i-Pr | 5-Cl | Br | CH₂—CHF | 30 | 523.6 | 524.0 | B |
| | | | | | | | (M) | |
| 111 | i-Pr | 2,5-Cl | Br | CH₂—CHF | 20 | 558.1 | 558.1 | A |
| | | | | | | | (M) | |
| 112 | Et | 2,5-Cl | Br | CH₂—CHF | 15 | 544.1 | 544.0 | B |
| | | | | | | | (M) | |
| 113 | Et | 2-Cl | Br | CH₂—CHF | 12 | 509.6 | 510.0 | B |
| | | | | | | | (M) | |
| 114 | Et | 5-Me | Br | (CH₂)₃ | 14 | 485.2 | 486.0 | B |
| 115 | i-Pr | 5-Me | Br | (CH₂)₃ | 30 | 499.2 | 500.0 | B |
| 116 | cyclobutyl | 5-Me | Br | (CH₂)₃ | 10 | 511.2 | 512.0 | B |
| 117 | Et | 2,5-Cl | Br | (CH₂)₃ | 5 | 540.1 | 540.0 | B |
| | | | | | | | (M) | |
| 118 | Et | 2,5-Cl | Br | CH═CH | 32 | 540.1 | 540.0 | C |
| | | | | | | | (M) | |
| 119 | i-Pr | 2,5-Cl | Br | CH═CH | 55 | 538.1 | 538.0 | C |
| | | | | | | | (M) | |
| 120 | Cyclobutyl | 2,5-Cl | Br | CH═CH | 43 | 550.1 | 550.0 | C |
| | | | | | | | (M) | |
| 121 | i-Pr | 5-Cl | Br | NH—CO—CH₂ | 28 | 534.6 | 533.9 | A |
| | | | | | | | (M − 1) | |

*Analyzed on HPLC-MS with alternating +/− API and equiped with different brands of 50 mm *2.1 mm, 5μ C8 columns. Eluted with 0.05% formic acid/ACN or 0.05% ammonium acetate/ACN
*MW calc. (molecular weight) is an isotopic average and the “found mass” is referring to the most abundant isotope detected in the LC-MS. The “found mass” refers to M + 1 unless specified otherwise.

### Example 122

### (S)-2-{2-(3,5-Dichloro-4-{[3-(ethylamino)-5-methylbenzyl]oxy}phenyl)acetylamino}-2-phenyl-acetic acid

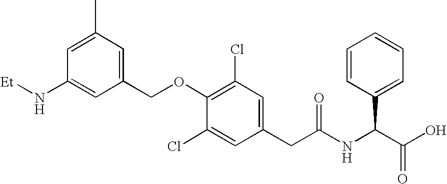

Sodium hydroxide (1 M aqueous, 2 mL) was added to the solution of methyl (S)-2-{2-(3,5-dichloro-4-{[3-(ethylamino)-5-methylbenzyl]oxy}phenyl)acetylamino}-2-phenyl-acetate (Description 13, 14 mg, 0.027 mmol) in 1,4 dioxane (0.2 mL). The mixture was stirred at room temperature overnight.

After acidification with hydrochloric acid (1 M), the product was extracted with ethyl acetate (3×10 mL). The combined organic phases were washed with brine, dried with magnesium sulphate and the solvent removed under vacuum. The residue was purified by semi-preparative HPLC (acid system) to give the title compound in 65% yield (8.5 mg) (M W=501.5). LC/MS (ESI): m/z 499.4 (M−2).

### Example 123

### Methyl (S)-2-{2-(3,5-dibromo-4-{[1,3-chloro-5-(ethylamino)benzyl]oxy}phenyl)acetylamino}-3-phenyl-propanoate

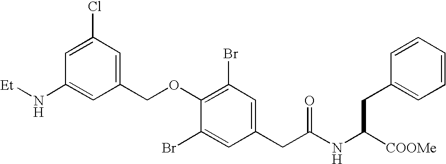

A solution of (3,5-dibromo-4-{[3-chloro-5-(ethylamino)benzyl]oxy}phenyl)acetic acid (35 mg, 0.07 mmol), bromotri(pyrrolidino)phosphonium hexafluorophosphate (PyBrOP) (39 mg, 0.084 mmol), 1-hydroxybenzotriazole hydrate (HOBT), (13 mg, 0.084 mmol), L-(+)-phenylalanine methyl ester hydrochloride (30 mg, 0.14 mmol) and diisopropyl ethyl amine (DIPEA), (47 mg, 0.37 mmol) in methylene chloride (1 ml) was stirred at room temperature for 16 h. The resulting reaction mixture was diluted with 1 M hydrochloric acid and extracted with dichloromethane (5×2 mL) using a phase separator. The combined organic phases were concentrated under vacuum and the residue was purified by flash chromatography (n-heptane/ethyl acetate 7:3) to afford methyl (S)-2-{2-(3,5-dibromo-4-{[3-chloro-5-(ethylamino)benzyl]oxy}phenyl)acetylamino}-3-phenyl-propanoate (MW=638.7). LC/MS (ESI): m/z 639.2 (M+1).

### Example 124

### (S)-2-{2-(3,5-Dibromo-4-{[3-chloro-5-(ethylamino)benzyl]oxy}phenyl)acetylamino}-3-phenyl-propanoic acid

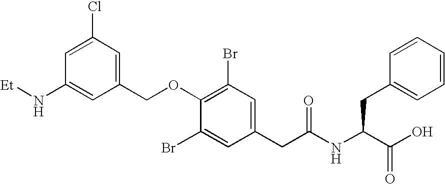

Lithium hydroxide (1 M aqueous, 1 mL) was added to the solution of methyl (S)-2-{2-(3,5-dibromo-4-{[3-chloro-5-(ethylamino)benzyl]oxy}phenyl)acetylamino}-3-phenyl-propanoate

(Example 123, 40 mg, 0.063 mmol) in tetrahydrofuran (1 mL). The mixture was stirred at room temperature for 2 h. After acidification with hydrochloric acid (1 M) the product was extracted with dichloromethane (5×5 mL) using a phase separator. The combined organic phases were reduced under vacuum and the resulting residue was filtrated through silica to give the title compound in 71% yield (31 mg) (M W=624.8). LC/MS (ESI): m/z 625:4 (M+1).

### Example 125

### 3-[3,5-Dichloro-4-({3-[(cyclopropyl)amino]benzyl}oxy)phenyl]propanoic acid

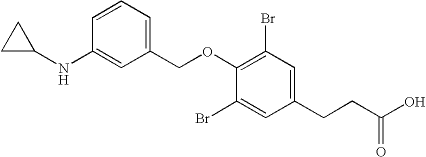

A mixture of methyl 3-[3,5-dichloro-4′-(3-iodobenzyloxy)phenyl]propanoate (Description 14, 120 mg, 0.26 mmol), cyclopropylamine (20 μL, 0.28 mmol), potassium carbonate (0.07 g, 0.51 mmol), copper iodide (0.025 g, 0.13 mmol) and proline (0.005 g, 0.15 mmol) in 2.3 mL of DMSO was heated at 60° C. for 12 h under nitrogen. The cooled mixture was filtered through a silica plug and concentrated. The residual oil was stirred in dioxane (2 mL) and potassium hydroxide (6 mL 2 M aq.) for 5 h. The residue was purified by semi-preparative-HPLC (Zorbax CombiHT (SB-C8) Mobil Phase: Solvent A. Water with 0.5% formic acid; μ50×21.2 mm, 5 Solvent B: acetonitrile. Gradient: 2 min 80% of A then over 8 min to 5% of A) to give 20 mg (20% yield) of the title compound 3-[3,5-dichloro-4-({3-[(cyclopropyl)amino]benzyl}oxy)phenyl]propanoic acid (M W=380.2). LC/MS (ESI): m/z 380.3 (M).

### Example 126

### {4-[(E)-2-(3-Amino-phenyl)-vinyl]-3,5-dibromo-benzyloxy}-acetic acid tert-butyl ester

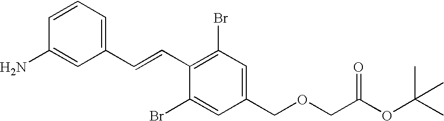

To {3,5-dibromo-4-[(E)-2-(3-nitro-phenyl)-vinyl]-benzyloxy}-acetic acid tert-butyl ester (Description 17, 0.010 g, 0.02 mmol) in ethanol (1 mL) was added SnCl₂ (0.02 g, 0.1 mmol). The mixture was stirred at reflux for 2 h. Ethyl acetate and saturated sodium carbonate were added and the product was extracted, dried and evaporated. The residue was purified on silica (dichloromethane) to give 0.009 g (100% yield) of {4-[(E)-2-(3-amino-phenyl)-vinyl]-3,5-dibromo-benzyloxy}-acetic acid tert-butyl ester.

### Abbreviations:

### SPE: Solid Phase Extraction

### NBS: N-Bromosuccinimide

ACN: acetonitrile

### Biological Assays

The utility of the compounds of the present invention can be evidenced by activity in at least one of the assays below.

### 1. Binding to Thyroid Hormone Receptors

The ability of compounds of the present invention to bind to thyroid hormone receptors was demonstrated and evaluated by the present inventors using a selection of the protocols found in the following scientific literature:
1) Barkhem, T.; Carlsson, B.; Simons, J.; Moeller, B.; Berkenstam, A.; Gustafsson, J.-Å.; Nilsson, S. High level expression of functional full-length human thyroid hormone receptor β1 in insect cells using a recombinant baculovirus. J. Steroid Biochem. Mol. Biol., 1991, 38, 667-75.
2) Carlsson, B.; Singh, B. N.; Temciuc, M.; Nilsson, S.; Li, Y.-L.; Mellin, C.; Malm, J. Synthesis and preliminary characterization of a novel antiarrhythmic compound (KB130015) with an improved toxicity profile compared with amiodarone. J. Med. Chem., 2002, 45, 623-630.
3) Liu Ye, yl-Lin Li, Karin Mellström, Charlotta Mell in, Lars-Göran Bladh, Konrad Koehler, Neeraj Garg, Ana Maria Garcia Collazo, Chris Litten, Bolette Husman, Karina Persson, Jan Ljunggren, Gary Grover, Paul G. Sleph, Rocco George, Johan Malm: Thyroid Receptor Ligands. I. Agonist Ligands Selective for the Thyroid Receptor β₁. J. Med. Chem., 2003, 45, 1580-1588.

The literature above contain not only protocols for binding experiments to the TR-receptor, but also vector constructs, generation of reporter cell lines and the corresponding assay procedures.

Compounds of the invention were found to exhibit binding affinities to the TR receptor in the range of from 1 nM to 500 nM.

### 2. Lipid Lowering Effects in Mice

The ability of a compound of the present invention to lower lipid levels in animals can be demonstrated and evaluated by those skilled in the art, using the following protocols:

### Cholesterol Fed C57BL/6J Mice

Weanling C57BL/6J mice were placed on a special diet protocol (Purina chow supplemented with 1.5% cholesterol, 15% saturated fat and 0.5% cholic acid) for two weeks before administration of drugs. The animals were housed at room temperature, 12:12 light dark cycle, and free access to food and water. On the day of treatment all animals were weighed before drug was administrated by intraperitoneal injection or by gavage. Compounds were administrated once daily for 5-10 days, at different concentrations (nmol/kg body weight), in suitable vehicle. On the last day of treatment, food was removed from the cages and the animals were fasted for at least 4 hours before termination of the study. Blood for serum or plasma was collected, and different organs were dissected and immediately frozen for later analyses. Blood and tissue lipid analyses were consecutively executed using commercial and readily available kits for the determination.

### Ob/ob Mice

The value of ob/ob mouse is well documented and appreciated by the one skilled in the art for monitoring “Metabolic Syndrome X”.

6-8 weeks old female ob/ob mice (i.e. leptin deficient mice) purchased from commercial supplier were used to characterize compounds binding to thyroid hormone receptors alpha (TRα) and beta (TRβ). The animals were weighed and randomly divided into different study groups, and kept for a minimum of 5 days to adapt to the new environment (animal facility). The animals were housed at room temperature, 12:12 light dark cycle, and free access to food and water. On the day of treatment all animals were weighed before drug was administrated by intraperitoneal injection or by gavage. Compounds were administrated once daily for 5-10 days, at different concentrations (nmol/kg body weight), in suitable vehicle. On the last day of treatment, food was removed from the cages and the animals were fasted for at least 4 hours before termination of the study. Blood for serum or plasma was collected, and different organs were dissected and immediately frozen for later analyses. Blood and tissue lipid analyses were consecutively executed using commercial and readily available kits for the determination.

Other assays that may be used for the demonstration of the effectiveness of the compounds of the invention include those described in the following references:
1) Liu Ye, yl-Lin Li, Karin Mellström, Charlotta Mellin, Lars-Göran Bladh, Konrad Koehler, Neeraj Garg, Ana Maria Garcia Collazo, Chris Litten, Bolette Husman, Karina Persson, Jan Ljunggren, Gary Grover, Paul G. Sleph, Rocco George, Johan Maim: Thyroid Receptor Ligands. I. Agonist Ligands Selective for the Thyroid Receptor β₁. J. Med. Chem., 2003, 45, 1580-1588.
2) Liu Ye, Johan Maim, yl-Lin Li, Lars-Göran Bladh, Karin Mellstrdm, Paul G. Sleph, Mark A. Smith, Rocco George, Björn Vennström, Kasim Mookhtiar, Ryan Horvath, Jessica Speelman, John D. Baxter, Gary J. Grover: Selective Thyroid Hormone Receptor-β Activation: A Strategy for Reduction of Weight, Cholesterol, and Lp(a) with Reduced Cardiovascular Liability. PNAS, 2003, 100, 10067-10072.

Other assays to determine thyroid receptor mediated activity of the test compounds include assays that demonstrate modulation of endogenous TR mediated transcription in cell culture systems; assays that demonstrate modulation of thyroid responsive tissue effects in rodents; assays for the identification of receptor surface conformation changes; and assays that demonstrate binding specificity to TR versus other nuclear receptors.

1. A compound of formula (I) or a pharmaceutically acceptable ester, amide, solvate or salt thereof, including a salt of such an ester or amide, and a solvate of such an ester, amide or salt, 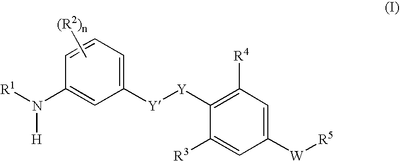
wherein:
R¹ is selected from hydrogen, C₁₋₈ alkyl, C₂₋₈ alkenyl, C₂₋₈ alkynyl, C₃₋₈ cycloalkyl and C₃₋₈ cycloalkyl-C₁₋₃ alkyl, said alkyl, alkenyl or alkynyl groups or portions of groups optionally being substituted with 1, 2 or 3 groups independently selected from halogen, hydroxy, methoxy, halomethoxy, dihalomethoxy, and trihalomethoxy; said cycloalkyl groups or portions of groups optionally being substituted with 1, 2 or 3 groups independently selected from halogen, hydroxy, C₁₋₄alkyl, C₂₋₄ alkenyl, C₂₋₄ alkynyl, methoxy, halomethoxy, dihalomethoxy, and trihalomethoxy, halo₁₄ alkyl, dihaloC₁₋₄ alkyl and trihaloC₁₋₄ alkyl;
Each R² is independently selected from halogen, mercapto, nitro, cyano, C₁₋₄ alkoxy, —C²R^{c}, —CONHR^{c}, —CHO, —SO₂R⁶, —SO₂NHR⁶, C₁₋₄ alkyl, C₂₋₄ alkenyl, C₂₋₄ alkynyl, NHR′ and N(R¹)₂, said alkyl, alkenyl, alkynyl or alkoxy groups optionally being substituted with 1, 2 or 3 groups selected from halogen, hydroxy, C₁₋₄ alkoxy, C₁₋₄ alkylthio, mercapto, nitro, cyano, halomethoxy, dihalomethoxy, and trihalomethoxy;
n is 0, 1, 2 or 3;
Y and Y′ together are —C(R^{a′})═C(R^{a′})—,
or alternatively Y and Y′ are independently selected from oxygen, sulphur and —CH(R^{a})—, with the proviso that at least one of Y and Y′ is —CH(R^{a})— and the further proviso that when one of Y and Y′ is oxygen or sulphur, then R^{a} is hydrogen, halogen, C₁₋₄ alkyl, C₂₋₄ alkenyl, C₂₋₄ alkynyl, fluoromethyl, difluoromethyl, or trifluoromethyl;
R^{a} is selected from hydrogen, halogen, hydroxy, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio and thiotrifluoromethyl;
R^{a′} is selected from hydrogen, halogen, mercapto, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy, methylthio, fluoromethylthio, difluoromethylthio and thiotrifluoromethyl;
R³ and R⁴ are independently selected from halogen, C₁₋₄ alkyl, C₂₋₄ alkenyl, C₂₋₄ alkynyl, fluoromethyl, difluoromethyl, trifluoromethyl, C₁₋₄ alkoxy, fluoromethoxy, difluoromethoxy, trifluoromethoxy, methylthio, fluoromethylthio, difluoromethylthio and trifluoromethylthio;
W is selected from C₁₋₃ alkylene, C₂₋₃ alkenylene, C₂₋₃ alkynylene, N(R^{b})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NH(CO)—C₀₋₃ alkylene, and C₁₋₃ alkyleneC(O)NH—C₁₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, phenyl, C₁₋₃ alkyl substituted with phenyl, haloC₁₋₃, alkyl, dihaloC₁₋₃ alkyl, trihaloC₁₋₃ alkyl, haloC₁₋₃ alkoxy, dihaloC₁₋₃ alkoxy, trigaloC₁₋₃ alkoxy and phenal substituted with 1, 2 or 3 halogen atoms;
R^{b} is selected from hydrogen, hydroxy, C₁₋₄ alkyl, C₂₋₄ alkenyl, C₂₋₄ alkynyl, C₁₋₄ alkoxy, fluoromethyl, difluoromethyl, trifluoromethyl, fluoromethoxy, difluoromethoxy, trifluoromethoxy;
R⁵ is selected from —CO₂R^{c}, —PO(OR^{c})₂—PO(OR^{c})NH₂, —SO₂OR^{c}, COCO₂R^{c}, CONR^{c}OR^{c}, —SO₂NHR^{c}, —NHSO₂R^{c′}, —CONHSO₂R^{c}, and —SO₂NHCOR^{c};
Each R^{c} is independently selected from hydrogen, C₁₋₄ alkyl, C₂₋₄ alkenyl, and C₂₋₄ alkynyl;
R^{c′} is selected from R^{c}, C₅₋₁₀ aryl and C₅₋₁₀ aryl substituted with 1, 2 or 3 groups independently selected from amino, hydroxy, halogen and C₁₋₄ alkyl;
with the proviso that when simultaneously n=0, R³═R⁴=Br, Y═O, Y′═CH₂, W═CH₂—CH₂ and R⁵═CO₂H, then R₁ is not ethyl or hydrogen.

2. A compound as claimed in claim 1 wherein R¹, R², n, R³, R⁴, and R⁵ are as defined in claim 1;
Y and Y′ are independently selected from oxygen, sulphur or —CH(R^{a})—, with the proviso that at least one of Y and Y′ is —CH(R^{a})— and the further proviso that when one of Y and Y′ is oxygen or sulphur, then R^{a} is hydrogen, halogen, C₁₋₄ alkyl, C₂₋₄ alkenyl, C₂₋₄ alkynyl, fluoromethyl, difluoromethyl, trifluoromethyl; and
W is selected from C₁₋₃ alkylene, C₂₋₃ alkenylene, C₂₋₃ alkynylene, N(R^{b})—C₁₋₃ alkylene, C(O)—C₁₋₃ alkylene, S—C₁₋₃ alkylene, O—C₁₋₃ alkylene, C(O)NH—C₁₋₃ alkylene, and NH(CO)—C₀₋₃ alkylene, said alkylene, alkenylene or alkynylene groups or portions of groups optionally being substituted with 1 or 2 groups selected from hydroxy, mercapto, amino, halo, C₁₋₃ alkyl, C₁₋₃ alkoxy, haloC₁₋₃ alkyl, dihalo C₁₋₃ alkyl, trihalo C₁₋₃ alkyl, halo C₁₋₃ alkoxy, dihalo C₁₋₃ alkoxy and trihalo C₁₋₃ alkoxy.

3. A compound as claimed in claim 1 which is a compound according to formula (Ia) or a pharmaceutically acceptable ester, amide, solvate or salt thereof, including a salt of such an ester or amide, and a solvate of such an ester, amide or salt, 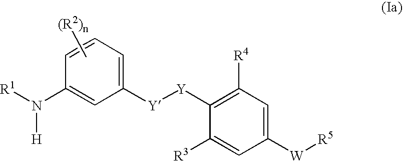
wherein:
n is 0, 1, 2 or 3;
When n=0 and simultaneously R³ and R⁴ are both Br, R¹ is selected from methyl, n-propyl, i-propyl, cyclobutyl, i-butyl, n-butyl and t-butyl, C₂₋₄ alkenyl, C₃₋₆ cycloalkyl-C₁₋₃ alkyl, said methyl, propyl, butyl, alkyl or alkenyl groups or portions of groups optionally being substituted with 1, 2 or 3 groups independently selected halogen, methoxy, halomethoxy, dihalomethoxy, and trihalomethoxy, said cycloalkyl groups or portions of groups originally being substituted with 1, 2 or 3 groups independently selected from halogen, methyl, ethyl, methoxy, halomethoxy, dihalomethoxy, and trihalomethoxy;
When n=0 and simultaneously R³ and R⁴ are not both Br, or when n=1, 2 or 3, R¹ is selected from hydrogen, C₁₋₄ alkyl, C₂₋₄ alkenyl and C₃₋₆ cycloalkyl-C₁₋₃ alkyl, said alkyl or alkenyl groups or portions of groups optionally being substituted with 1, 2 or 3 groups independently selected from halogen, methoxy, halomethoxy, dihalomethoxy, and trihalomethoxy; said cycloalkyl groups or portions of groups originally being substituted with 1, 2 or 3 groups independently selected from halogen, methyl, ethyl, methoxy, halomethoxy, dihalomethoxy, and trihalomethoxy;
Each R² is independently selected from halogen, C₁₋₂ alkyl, C₂₋₃ alkenyl, C₂₋₃ alkynyl, C₁₋₂ alkoxy, haloC₁₋₂ alkyl, dihalo C₁₋₂ alkyl, trihalo C₁₋₂ alkyl;
Y and Y′ together are —C(R^{a′})═C(R^{a′}),
or alternatively Y is O or S, and Y′ is —CH(R^{a})—;
R^{a} is selected from hydrogen, halogen, C₁₋₂ alkyl, fluoromethyl, difluoromethyl and trifluoromethyl;
R^{a}, is selected from hydrogen, halogen, C₁₋₂ alkyl, fluoromethyl, difluoromethyl and trifluoromethyl;
R³ and R⁴ are independently selected from halogen, C₁₋₄ alkyl, fluoromethyl, difluoromethyl, and trifluoromethyl;
W is selected from C₁₋₃ alkylene, C₂₋₃ alkenylene, O—C₁₋₃ alkylene, C₁₋₃ alkylene-O—C₁₋₃ alkylene, C(O)—C₁₋₂ alkylene, C(O)NH—C₁₋₂ alkylene AND NH(CO)—C₁₋₂ alkylene; the alkylene group or portion of a group optionally being substituted with one or more halo groups.
R⁵ is selected from —CO₂R^{c}, —PO(OR^{c})₂—SO₂OR^{c}, —NHSO₂R^{c′}, —COCO₂R^{c}, CONR^{c}OR^{c};
Each R^{c} is independently selected from ethyl, methyl and hydrogen; and
R^{c′} is selected from R^{c}, phenyl and phenyl substituted with 1, 2 or 3 groups independently selected from amino, hydroxyl, halogen or methyl.

4. A compound as claimed in claim 1 which is a compound according to formula (Ib) or a pharmaceutically acceptable ester, amide, solvate or salt thereof, including a salt of such an ester or amide, and a solvate of such an ester, amide or salt, 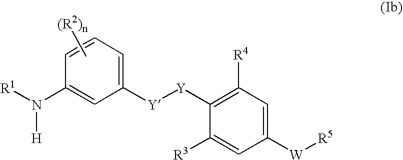
wherein:
n is 0, 1, 2 or 3;
When n=0 and simultaneously R³ and R⁴ are both Br, R¹ is selected from methyl, n-propyl, i-propyl, cyclobutyl, i-butyl, n-butyl and t-butyl, C₂₋₄ alkenyl, C₃₋₆ cycloalkyl-C₁₋₃ alkyl;
When n=0 and simultaneously R³ and R⁴-are not both Br, or when n=1, 2 or 3, R¹ is selected from hydrogen, C₁₋₄ alkyl, C₂₋₄ alkenyl and C₃₋₆ cycloalkyl-C₁₋₃ alkyl;
Each R² is independently selected from halogen, C₁₋₂ alkyl, C₂₋₃ alkenyl, C₂₋₃ alkynyl, C₁₋₂ alkoxy, haloC₁₋₂ alkyl, dihalo C₁₋₂ alkyl, trihalo C₁₋₂ alkyl;
Y and Y′ together are —C(R^{a′})═C(R^{a′})—,
or alternatively Y is O, and Y′ is —CH(R^{a})—;
R^{a} is selected from hydrogen, halogen, and C₁₋₂ alkyl;
R^{a′} is selected from hydrogen, halogen, and C₁₋₂ alkyl;
R³ and R⁴ are independently selected from halogen, C₁₋₄ alkyl, fluoromethyl, difluoromethyl, and trifluoromethyl;
W is selected from C₁₋₃ alkylene, C₂₋₃ alkenylene, O—C₁₋₃ alkylene, C₁₋₃ alkylene-O—C₁₋₃ alkylene, C(O)NH—C₁₋₂ alkylene and NH(CO)—C₁₋₂ alkylene; the alkylene group or portion of a group optionally being substituted with one or more halo groups.
R⁵ is —CO₂R^{c};
Each R^{c} is independently selected from ethyl, methyl and hydrogen.

5. A compound as claimed in claim 1 for use as a medicament.

6. A compound as claimed in claim 1 or a pharmaceutically acceptable ester, amide, solvate or salt thereof, including a salt of such ester or amide, and a solvate of such an ester, amide or salt, for use in the treatment or prophylaxis of a condition associated with a disease or disorder associated with thyroid receptor activity.

7. A method for the treatment or prophylaxis of a disease or disorder associated with thyroid receptor activity in a mammal, which comprises administering to the mammal a therapeutically effective amount of a compound of formula (I) as defined in claim 1 or a pharmaceutically acceptable ester, amide, solvate or salt thereof, including a salt of such an ester or amide, and a solvate of such an ester, amide or salt.

8. (canceled)

9. A pharmaceutical formulation comprising a compound as defined in claim 1 or a pharmaceutically acceptable ester, amide, solvate or salt thereof, including a salt of such an ester or amide, and a solvate of such an ester, amide or salt, and a pharmaceutically acceptable excipient.

10. A pharmaceutical formulation as claimed in claim 9 further comprising an additional therapeutic agent selected from cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

11. (canceled)

12. A method of discovering a ligand of the thyroid hormone receptor which comprising use of a compound as defined in claim 1 or a compound as defined in claim 1 in labelled form, as a reference compound.

13. A compound as claimed in claim 6, wherein the condition associated with a disease or disorder associated with thyroid receptor activity is selected from (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

14. A method for preparing a compound of formula (I) as described in claim 1 in which R¹ is not H, comprising a step of reacting
a compound of formula (II) 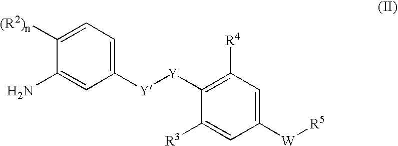
Wherein R², n, Y′, Y, R³, R⁴, W and R⁵ are as defined in claim 1
with a compound of formula R^{1′}—CHO or R^{1″}—C(O)—R^{1′″}, wherein R^{1′}, R^{1″}, and R^{1′″} are chosen such that the product compound comprises the group R¹ as defined in claim 1, optionally in the presence of a reducing agent, followed optionally by interconversion to another compound as described in claim 1.

15. A method for preparing a compound of formula (I) as descried in claim 1 in which R¹ is hydrogen, comprising a step of reacting
a compound of formula (III) 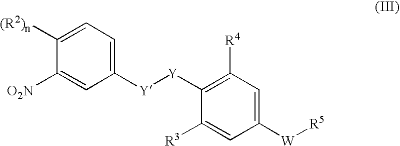
Wherein R², n, Y′, Y, R³, R⁴, W and R⁵ are as defined in claim 1
with a suitable reducing agent, followed optionally by interconversion to another compound as described in claim 1.

16. A pharmaceutical composition as claimed in claim 10 wherein the additional therapeutic agent is a hypolipidemic agent selected from the group consisting of an acyl coenzyme A cholesterol acyltransferase (ACAT) inhibitor, a microsomal triglyceride transfer protein (MTP) inhibitor, a cholesterol ester transfer protein (CETP) inhibitor, a ileal bile acid transporter (IBAT) inhibitor, any cholesterol absorption inhibitor, a 3-hydroxy-3-methylglutaryl coenzyme A (HMG-CoA) reductase inhibitor, a squalene synthetase inhibitor, a bile acid sequestrant, a peroxisome proliferator-activator receptor (PPAR)-alpha agonist, a peroxisome proliferator-activator receptor (PPAR)-delta agonist, any peroxisome proliferator-activator receptor (PPAR)-gamma/delta dual agonist, any peroxisomeproliferator-activator receptor (PPAR)-alpha/delta dual agonist, a nicotinic acid or a derivative thereof, and a thiazolidinedione or a derivative thereof.

17. A pharmaceutical composition as claimed in claim 10 wherein the additional therapeutic agent is a hypolipidemic agent selected from the group consisting of ezetimibe, simvastatin, atorvastatin, rosuvastatin, cerivastatin, fluvastatin, lovastatin, pravastatin, fenofibrate, gemfibrozil and bezafibrate.

18. A pharmaceutical composition as claimed in claim 10 wherein the additional therapeutic agent is an antidiabetic agent selected from the group consisting of a biguanide, a glucosidase inhibitor, a meglitinide, a sulfonylurea, a thiazolidinedione, a peroxisome proliferator-activator receptor (PPAR)-alpha agonist, a peroxisome proliferator-activator receptor (PPAR)-gamma agonist, a peroxisome proliferator-activator receptor (PPAR)alpha/gamma dual agonist, a sodium glucose co-transporter (SGLT) 1, 2 or 3 inhibitor, a glycogen phosphorylase inhibitor, an aP2 inhibitor, a glucagon-like peptide-1 (GLP-1), a dipeptidyl peptidase IV inhibitor, a glucocorticoid (GR) antagonist and insulin.

19. A pharmaceutical composition as claimed in claim 10 wherein the additional therapeutic agent is an antidiabetic agent selected from the group consisting of metformin, glyburide, glimepiride, glipyride, glipizide, chlorpropamide, gliclazide, acarbose, miglitol, troglitazone, pioglitazone, englitazone, darglitazone, rosiglitazone and insulin.

20. A pharmaceutical composition as claimed in claim 10 wherein the additional therapeutic agent is an anti-obesity agent is selected from the group consisting of an aP2 inhibitor, a peroxisome proliferator-activator receptor (PPAR) gamma antagonist, a peroxisome proliferator-activator receptor (PPAR) delta agonist, a beta-3 adrenergic agonist, a lipase inhibitor, a serotonin reuptake inhibitor and an anorectic agent.
